SEQUENCE LISTING (313 sequences)

SEQ ID NO: 1 - protein, 16 aa, Plasmodium falciparum
NANPNANPNANPNANP

SEQ ID NO: 2 - protein, 20 aa, Plasmodium falciparum
EYLNKIQNSLSTEWSPASVT

SEQ ID NO: 3 - protein, 15 aa, Streptococcus pneumoniae
KLEELSDKIDELDAE

SEQ ID NO: 4 - protein, 35 aa, Streptococcus pneumoniae
QKKYDEDQKKTEEKAALEKAASEEMDKAVAAVQQA

SEQ ID NO: 5 - protein, 27 aa, Cryptosporidium parvum
QDKPADAPAAEAPAAEPAAQQDKPADA

SEQ ID NO: 6 - protein, 17 aa, Human immunodeficiency virus type 1
RKRIHIGPGRAFYITKN

SEQ ID NO: 7 - protein, 31 aa, Foot-and-mouth disease virus
YNGECRYNRNAVPNLRGDLQVLAQKVARTLP

SEQ ID NO: 8 - protein, 10 aa, Influenza A virus
YRNLLWLTEK

SEQ ID NO: 9 - protein, 23 aa, Influenza A virus
SLLTEVETPIRNEWGCRCNGSSD

SEQ ID NO: 10 - protein, 23 aa, Influenza A virus
SLLTEVETPIRNEWGCRCNDSSD

SEQ ID NO: 11 - protein, 142 aa, Yersinia pestis
DILKVIVDSMNHHGDARSKLREELAELTAELKIYSVIQAEINKHLSSSGTINIHDKSINLMDKNLYGYTDEEIFKASAEYKILEKMPQTTIQVDGSEKKIVSIKDFLGSENKRTGALGNLKNSYSYNKDNNELSHFATTCSD

SEQ ID NO: 12 - protein, 19 aa, Haemophilus influenzae
CSSSNNDAAGNGAAQFGGY

SEQ ID NO: 13 - protein, 11 aa, Haemophilus influenzae
NKLGTVSYGEE

SEQ ID NO: 14 - protein, 16 aa, Haemophilus influenzae
NDEAAYSKNRRAVLAY

SEQ ID NO: 15 - protein, 28 aa, Moraxella catarrhalis
LDIEKDKKKRTDEQLQAELDDKYAGKGY

SEQ ID NO: 16 - protein, 28 aa, Moraxella catarrhalis
LDIEKNKKKRTEAELQAELDDKYAGKGY

SEQ ID NO: 17 - protein, 27 aa, Moraxella catarrhalis
IDIEKKGKIRTEALLAELNKDYPGQGY

SEQ ID NO: 18 - protein, 25 aa, Porphyromonas gingivalis
GVSPKVCKDVTVEGSNEFAPVQNLT

SEQ ID NO: 19 - protein, 20 aa, Porphyromonas gingivalis
RIQSTWRQKTVDLPAGTKYV

SEQ ID NO: 20 - protein, 21 aa, Trypanosoma cruzi
KAAIAPAKAAAAPAKAATAPA

SEQ ID NO: 21 - protein, 24 aa, Plasmodium falciparum
NANPNVDPNANPNANPNANPNVDP

SEQ ID NO: 22 - protein, 20 aa, Plasmodium falciparum
NANPNVDPNANPNANPNANP

SEQ ID NO: 23 - protein, 20 aa, Plasmodium falciparum
NANPNANPNANPNVDPNANP

SEQ ID NO: 24 - protein, 28 aa, Plasmodium falciparum
NANPNVDPNANPNANPNANPNVDPNANP

SEQ ID NO: 25 - protein, 20 aa, Plasmodium falciparum
NPNVDPNANPNANPNANPNV

SEQ ID NO: 26 - protein, 22 aa, Plasmodium falciparum
NPNVDPNANPNANPNANPNVDP

SEQ ID NO: 27 - protein, 24 aa, Plasmodium falciparum
NPNVDPNANPNANPNANPNVDPNA

SEQ ID NO: 28 - protein, 18 aa, Plasmodium falciparum
NVDPNANPNANPNANPNV

SEQ ID NO: 29 - protein, 20 aa, Plasmodium falciparum
NVDPNANPNANPNANPNVDP

SEQ ID NO: 30 - protein, 22 aa, Plasmodium falciparum
NVDPNANPNANPNANPNVDPNA

SEQ ID NO: 31 - protein, 16 aa, Plasmodium falciparum
DPNANPNANPNANPNV

SEQ ID NO: 32 - protein, 18 aa, Plasmodium falciparum
DPNANPNANPNANPNVDP

SEQ ID NO: 33 - protein, 20 aa, Plasmodium falciparum
DPNANPNANPNANPNVDPNA

SEQ ID NO: 34 - protein, 19 aa, Plasmodium vivax
GDRADGQPAGDRADGQPAG

SEQ ID NO: 35 - protein, 18 aa, Plasmodium vivax
RADDRAAGQPAGDGQPAG

SEQ ID NO: 36 - protein, 18 aa, Plasmodium vivax
ANGAGNQPGANGAGDQPG

SEQ ID NO: 37 - protein, 18 aa, Plasmodium vivax
ANGADNQPGANGADDQPG

SEQ ID NO: 38 - protein, 18 aa, Plasmodium vivax
ANGAGNQPGANGADNQPG

SEQ ID NO: 39 - protein, 18 aa, Plasmodium vivax
ANGADNQPGANGADDQPG

SEQ ID NO: 40 - protein, 22 aa, Plasmodium vivax
APGANQEGGAAAPGANQEGGAA

SEQ ID NO: 41 - protein, 16 aa, Plasmodium berghei
DPPPPNPNDPPPPNPN

SEQ ID NO: 42 - protein, 24 aa, Plasmodium yoelii
QGPGAPQGPGAPQGPGAPQGPGAP

SEQ ID NO: 43 - protein, 15 aa, Streptococcus sobrinus
KPRPIYEAKLAQNQK

SEQ ID NO: 44 - protein, 16 aa, Streptococcus sobrinus
AKADYEAKLAQYEKDL

SEQ ID NO: 45 - protein, 9 aa, Shigella flexneri
KDRTLIEQK

SEQ ID NO: 46 - protein, 15 aa, respiratory syncytial virus
CSICSNNPTCWAICK

SEQ ID NO: 47 - protein, 25 aa, Entamoeba histolytica
VECASTVCQNDNSCPIIADVEKCNQ

SEQ ID NO: 48 - protein, 34 aa, Schistosoma japonicum
DLQSEISLSLENGELIRRAKSAESLASELQRRVD

SEQ ID NO: 49 - protein, 34 aa, Schistosoma mansoni
DLQSEISLSLENSELIRRAKAAESLASDLQRRVD

SEQ ID NO: 50 - protein, 16 aa, Human immunodeficiency virus
GPKEPFRDYVDRFYKC

SEQ ID NO: 51 - protein, 17 aa, Corynebacterium diphtheriae
FQVVHNSYNRPAYSPGC

SEQ ID NO: 52 - protein, 25 aa, Borrelia burgdorferi
VEIKEGTVTLKREIDKNGKVTVSLC

SEQ ID NO: 53 - protein, 19 aa, Borrelia burgdorferi
TLSKNISKSGEVSVELNDC

SEQ ID NO: 54 - protein, 11 aa, Influenza A virus
SSVSSFERFEC

SEQ ID NO: 55 - protein, 21 aa, Trypanosoma cruzi
SHNFTLVASVIIEEAPSGNTC

SEQ ID NO: 56 - protein, 16 aa, Plasmodium falciparum
SVQIPKVPYPNGIVYC

SEQ ID NO: 57 - protein, 16 aa, Plasmodium falciparum
DFNHYYTLKTGLEADC

SEQ ID NO: 58 - protein, 18 aa, Plasmodium falciparum
PSDKHIEQYKKIKNSISC

SEQ ID NO: 59 - protein, 20 aa, Plasmodium falciparum
EYLNKIQNSLSTEWSPCSVT

SEQ ID NO: 60 - protein, 19 aa, Plasmodium vivax
YLDKVRATVGTEWTPCSVT

SEQ ID NO: 61 - protein, 16 aa, Streptococcus sobrinus
KPRPIYEAKLAQNQKC

SEQ ID NO: 62 - protein, 17 aa, Streptococcus sobrinus
AKADYEAKLAQYEKDLC

SEQ ID NO: 63 - protein, 16 aa, Lymphocytic choriomeningitis virus
RPQASGVYMGNLTAQC

SEQ ID NO: 64 - protein, 16 aa, Clostridium tetani
QYIKANSKFIGITELC

SEQ ID NO: 65 - DNA, 18 bp, plasmid pKK223
ggtgcatgcaaggagatg

SEQ ID NO: 66 - DNA, 55 bp, plasmid pKK223
gcgaagcttcggatcccatggttttttcctccttatgtgaaattgttatccgctc

SEQ ID NO: 67 - DNA, 24 bp, Hepatitis B virus
ttgggccatggacatcgaccctta

SEQ ID NO: 68 - DNA, 29 bp, Hepatitis B virus
gcggaattccttccaaattaacacccacc

SEQ ID NO: 69 - DNA, 38 bp, Hepatitis B virus
cgcgaattcaaaaagagctcgatccagcgtctagagac

SEQ ID NO: 70 - DNA, 31 bp, Hepatitis B virus
cgcaagcttaaacaacagtagtctccggaag

SEQ ID NO: 71 - DNA, 40 bp, Artificial Sequence
  Description of Artificial Sequence human cytochrome 450
cgagctgtacatttgcgttttcgtctagctgtttttcttg

SEQ ID NO: 72 - DNA, 31 bp, Hepatitis B virus
gcggaattccatcttccaaattaacacccac

SEQ ID NO: 73 - DNA, 39 bp, Hepatitis B virus
cgcgaattcaaaaagagctcccagcgtctagagacctag

SEQ ID NO: 74 - DNA, 39 bp, Artificial Sequence
  Description of Artificial Sequence human cytochrome P450
caagaaaaacagctagacgaaaacgcaaatgtacagctc

SEQ ID NO: 75 - DNA, 42 bp, Hepatitis B virus
cgcaagcttagagctcttgaattccaacaacagtagtctccg

SEQ ID NO: 76 - DNA, 28 bp, Hepatitis B virus
cgcgagctcccagcgtctagagacctag

SEQ ID NO: 77 - DNA, 17 bp, plasmid pKK223
gtatcaggctgaaaatc

SEQ ID NO: 78 - protein, 19 aa, Plasmodium falciparum
INANPNANPNANPNANPEL

SEQ ID NO: 79 - DNA, 57 bp, Plasmodium falciparum
aattaacgctaatccgaacgctaatccgaacgctaatccgaacgctaatccggagct

SEQ ID NO: 80 - DNA, 49 bp, Plasmodium falciparum
ccggattagcgttcggattagcgttcggattagcgttcggattagcgtt

SEQ ID NO: 81 - protein, 31 aa, Plasmodium falciparum
INANPNVDPNANPNANPNANPNVDPNANPEL

SEQ ID NO: 82 - DNA, 93 bp, Plasmodium falciparum
aattaacgctaatccgaacgttgacccgaacgctaatccgaacgctaatccgaacgctaatccgaacgttgacccgaacgctaatccggagct

SEQ ID NO: 83 - DNA, 91 bp, Plasmodium falciparum
ggagctccggattagcgttcgggtcaacgttcggattagcgttcggattagcgttcggattagcgttcgggtcaacgttcggattagcgtt

SEQ ID NO: 84 - protein, 23 aa, Plasmodium falciparum
INANPNVDPNANPNANPNANPEL

SEQ ID NO: 85 - DNA, 69 bp, Plasmodium falciparum
aattaacgcgaatccgaacgtggatccgaatgccaaccctaacgccaacccaaatgcgaacccagagct

SEQ ID NO: 86 - DNA, 61 bp, Plasmodium falciparum
ctgggttcgcatttgggttggcgttagggttggcattcggatccacgttcggattcgcgtt

SEQ ID NO: 87 - protein, 23 aa, Plasmodium falciparum
INANPNANPNANPNVDPNANPEL

SEQ ID NO: 88 - DNA, 69 bp, Plasmodium falciparum
aattaacgcgaatccgaatgccaaccctaacgccaacccaaacgtggatccgaatgcgaacccagagct

SEQ ID NO: 89 - DNA, 61 bp, Plasmodium falciparum
ctgggttcgcattcggatccacgtttgggttggcgttagggttggcattcggattcgcgtt

SEQ ID NO: 90 - protein, 31 aa, Plasmodium falciparum; identical to SEQ ID NO: 81

SEQ ID NO: 91 - DNA, 93 bp, Plasmodium falciparum
aattaacgcgaatccgaacgtggatccaaatgccaaccctaacgctaatccaaacgccaacccgaatgttgaccccaatgccaatccggagct

SEQ ID NO: 92 - DNA, 85 bp, Plasmodium falciparum
ccggattggcattggggtcaacattcgggttggcgtttggattagcgttagggttggcatttggatccacgttcggattcgcgtt

SEQ ID NO: 93 - protein, 23 aa, Plasmodium falciparum
INPNVDPNANPNANPNANPNVEL

SEQ ID NO: 94 - DNA, 69 bp, Plasmodium falciparum
aattaatccgaacgtggatccaaatgccaaccctaacgctaatccaaacgccaacccgaatgttgagct

SEQ ID NO: 95 - DNA, 61 bp, Plasmodium falciparum
caacattcgggttggcgtttggattagcgttagggttggcatttggatccacgttcggatt

SEQ ID NO: 96 - protein, 25 aa, Plasmodium falciparum
INPNVDPNANPNANPNANPNVDPEL

SEQ ID NO: 97 - DNA, 75 bp, Plasmodium falciparum
aattaatccgaacgtggatccaaatgccaaccctaacgctaatccaaacgccaacccgaatgttgaccctgagct

SEQ ID NO: 98 - DNA, 67 bp, Plasmodium falciparum
cagggtcaacattcgggttggcgtttggattagcgttagggttggcatttggatccacgttcggatt

SEQ ID NO: 99 - protein, 27 aa, Plasmodium falciparum
INPNVDPNANPNANPNANPNVDPNAEL

SEQ ID NO: 100 - DNA, 81 bp, Plasmodium falciparum
aattaatccgaacgtggatccaaatgccaaccctaacgctaatccaaacgccaacccgaatgttgaccctaatgctgagct

SEQ ID NO: 101 - DNA, 73 bp, Plasmodium falciparum
cagcattagggtcaacattcgggttggcgtttggattagcgttagggttggcatttggatccacgttcggatt

SEQ ID NO: 102 - protein, 21 aa, Plasmodium falciparum
INVDPNANPNANPNANPNVEL

SEQ ID NO: 103 - DNA, 63 bp, Plasmodium falciparum
aattaacgtggatccaaatgccaaccctaacgctaatccaaacgccaacccgaatgttgagct

SEQ ID NO: 104 - DNA, 55 bp, Plasmodium falciparum
caacattcgggttggcgtttggattagcgttagggttggcatttggatccacgtt

SEQ ID NO: 105 - protein, 23 aa, Plasmodium falciparum
INVDPNANPNANPNANPNVDPEL

SEQ ID NO: 106 - DNA, 69 bp, Plasmodium falciparum
aattaacgtggatccaaatgccaaccctaacgctaatccaaacgccaacccgaatgttgaccctgagct

SEQ ID NO: 107 - DNA, 61 bp, Plasmodium falciparum
cagggtcaacattcgggttggcgtttggattagcgttagggttggcatttggatccacgtt

SEQ ID NO: 108 - protein, 25 aa, Plasmodium falciparum
INVDPNANPNANPNANPNVDPNAEL

SEQ ID NO: 109 - DNA, 75 bp, Plasmodium falciparum
aattaacgtggatccaaatgccaaccctaacgctaatccaaacgccaacccgaatgttgaccctaatgctgagct

SEQ ID NO: 110 - DNA, 67 bp, Plasmodium falciparum
cagcattagggtcaacattcgggttggcgtttggattagcgttagggttggcatttggatccacgtt

SEQ ID NO: 111 - protein, 19 aa, Plasmodium falciparum
IDPNANPNANPNANPNVEL

SEQ ID NO: 112 - DNA, 57 bp, Plasmodium falciparum
aattgatccaaatgccaaccctaacgctaatccaaacgccaacccgaatgttgagct

SEQ ID NO: 113 - DNA, 49 bp, Plasmodium falciparum
caacattcgggttggcgtttggattagcgttagggttggcatttggatc

SEQ ID NO: 114 - protein, 21 aa, Plasmodium falciparum
IDPNANPNANPNANPNVDPEL

SEQ ID NO: 115 - DNA, 63 bp, Plasmodium falciparum
aattgatccaaatgccaaccctaacgctaatccaaacgccaacccgaatgttgaccctgagct

SEQ ID NO: 116 - DNA, 55 bp, Plasmodium falciparum
cagggtcaacattcgggttggcgtttggattagcgttagggttggcatttggatc

SEQ ID NO: 117 - protein, 23 aa, Plasmodium falciparum
IDPNANPNANPNANPNVDPNAEL

SEQ ID NO: 118 - DNA, 69 bp, Plasmodium falciparum
aattgatccaaatgccaaccctaacgctaatccaaacgccaacccgaatgttgaccctaatgccgagct

SEQ ID NO: 119 - DNA, 61 bp, Plasmodium falciparum
cggcattagggtcaacattcgggttggcgtttggattagcgttagggttggcatttggatc

SEQ ID NO: 120 - protein, 21 aa, Plasmodium falciparum
IEYLNKIQNSLSTEWSPCSVT

SEQ ID NO: 121 - DNA, 69 bp, Plasmodium falciparum
aattgaatatctgaacaaaatccagaactctctgtccaccgaatggtctccgtgctccgttacctagta

SEQ ID NO: 122 - DNA, 69 bp, Plasmodium falciparum
agcttactaggtaacggagcacggagaccattcggtggacagagagttctggattttgttcagatattc

SEQ ID NO: 123 - protein, 24 aa, Plasmodium vivax
IPAGDRADGQPAGDRAAGQPAGEL

SEQ ID NO: 124 - DNA, 72 bp, Plasmodium vivax
aattccggctggtgaccgtgcagatggccagccagcgggtgaccgcgctgcaggccagccggctggcgagct

SEQ ID NO: 125 - DNA, 64 bp, Plasmodium vivax
cgccagccggctggcctgcagcgcggtcacccgctggctggccatctgcacggtcaccagccgg

SEQ ID NO: 126 - protein, 21 aa, Plasmodium vivax
IDRAAGQPAGDRADGQPAGEL

SEQ ID NO: 127 - DNA, 63 bp, Plasmodium vivax
aattgacagagcagccggacaaccagcaggcgatcgagcagacggacagcccgcaggggagct

SEQ ID NO: 128 - DNA, 55 bp, Plasmodium vivax
cccctgcgggctgtccgtctgctcgatcgcctgctggttgtccggctgctctgtc

SEQ ID NO: 129 - protein, 21 aa, Plasmodium vivax
IANGAGNQPGANGAGDQPGEL

SEQ ID NO: 130 - DNA, 63 bp, Plasmodium vivax
aattgcgaacggcgccggtaatcagccgggggcaaacggcgcgggtgatcaaccaggggagct

SEQ ID NO: 131 - DNA, 55 bp, Plasmodium vivax
cccctggttgatcacccgcgccgtttgcccccggctgattaccggcgccgttcgc

SEQ ID NO: 132 - protein, 21 aa, Plasmodium vivax
IANGADNQPGANGADDQPGEL

SEQ ID NO: 133 - DNA, 63 bp, Plasmodium vivax
aattgcgaacggcgccgataatcagccgggtgcaaacggggcggatgaccaaccaggcgagct

SEQ ID NO: 134 - DNA, 55 bp, Plasmodium vivax
cgcctggttggtcatccgccccgtttgcacccggctgattatcggcgccgttcgc

SEQ ID NO: 135 - protein, 39 aa, Plasmodium vivax
IANGAGNQPGANGAGDQPGANGADNQPGANGADDQPGEL

SEQ ID NO: 136 - DNA, 117 bp, Plasmodium vivax
aattgcgaacggcgccggtaatcagccgggagcaaacggcgcgggggatcaaccaggcgccaatggtgcagacaaccagcctggggcgaatggagccgatgaccaacccggcgagct

SEQ ID NO: 137 - DNA, 109 bp, Plasmodium vivax
cgccgggttggtcatcggctccattcgccccaggctggttgtctgcaccattggcgcctggttgatcccccgcgccgtttgctcccggctgattaccggcgccgttcgc

SEQ ID NO: 138 - protein, 25 aa, Plasmodium vivax
IAPGANQEGGAAAPGANQEGGAAEL

SEQ ID NO: 139 - DNA, 75 bp, Plasmodium vivax
aattgcgccgggcgccaaccaggaaggtggggctgcagcgccaggagccaatcaagaaggcggtgcagcggagct

SEQ ID NO: 140 - DNA, 67 bp, Plasmodium vivax
ccgctgcaccgccttcttgattggctcctggcgctgcagccccaccttcctggttggcgcccggcgc

SEQ ID NO: 141 - protein, 21 aa, Plasmodium vivax
IEYLDKVRATVGTEWTPCSVT

SEQ ID NO: 142 - DNA, 69 bp, Plasmodium vivax
aattgaatatctggataaagtgcgtgcgaccgttggcacggaatggactccgtgcagcgtgacctaata

SEQ ID NO: 143 - DNA, 69 bp, Plasmodium vivax
agcttattaggtcacgctgcacggagtccattccgtgccaacggtcgcacgcactttatccagatattc

SEQ ID NO: 144 - protein, 10 aa, Plasmodium falciparum
TVSAPSWETS

SEQ ID NO: 145 - DNA, 42 bp, Plasmodium falciparum
gccaagcttactaggtaacggaggccggagaccattcggtgg

SEQ ID NO: 146 - DNA, 44 bp, Plasmodium vivax
cgcgaattcaagcgaacggcgccgataatcagccggcgggtgca

SEQ ID NO: 147 - protein, 8 aa, Hepatitis B virus
CVVTTEPL

SEQ ID NO: 148 - DNA, 37 bp, Hepatitis B virus
cgcaagcttactagcaaacaacagtagtctccggaag

SEQ ID NO: 149 - protein, 7 aa, Hepatitis B virus
PLTSLIP

SEQ ID NO: 150 - DNA, 32 bp, Hepatitis B virus
cgcaagcttacggaagtgttgataggataggg

SEQ ID NO: 151 - protein, 8 aa, Hepatitis B virus
TSLIPANP

SEQ ID NO: 152 - DNA, 34 bp, Hepatitis B virus
cgcaagcttatgttgataggataggggcatttgg

SEQ ID NO: 153 - protein, 7 aa, Hepatitis B virus
LIPANPP

SEQ ID NO: 154 - DNA, 31 bp, Hepatitis B virus
cgcaagcttataggataggggcatttggtgg

SEQ ID NO: 155 - protein, 6 aa, Hepatitis B virus
IPANPP

SEQ ID NO: 156 - DNA, 28 bp, Hepatitis B virus
gcgaagcttagataggggcatttggtgg

SEQ ID NO: 157 - protein, 6 aa, Hepatitis B virus
PANPPR

SEQ ID NO: 158 - DNA, 28 bp, Hepatitis B virus
cgcaagcttaaggggcatttggtggtct

SEQ ID NO: 159 - protein, 7 aa, Hepatitis B virus
CPANPPR

SEQ ID NO: 160 - protein, 7 aa, Hepatitis B virus
ANPPRYA

SEQ ID NO: 161 - DNA, 31 bp, Hepatitis B virus
gcgaagcttagcaaggggcatttggtggtct

SEQ ID NO: 162 - DNA, 30 bp, Hepatitis B virus
gcgaagcttaggcatttggtggtctatagc

SEQ ID NO: 163 - protein, 8 aa, Hepatitis B virus
CANPPRYA

SEQ ID NO: 164 - DNA, 32 bp, Hepatitis B virus
gcgaagcttagcaggcatttggtggtctataa

SEQ ID NO: 165 - protein, 7 aa, Hepatitis B virus
NPPRYAP

SEQ ID NO: 166 - DNA, 31 bp, Hepatitis B virus
cgcaagcttaatttggtggtctataagctgg

SEQ ID NO: 167 - protein, 8 aa, Plasmodium falciparum
NANPNVDP

SEQ ID NO: 168 - protein, 6 aa, Homo sapiens
NYKKPK

SEQ ID NO: 169 - protein, 7 aa, Hepatitis B virus
KRGPRTH

SEQ ID NO: 170 - protein, 21 aa, Homo sapiens
LHPDETKNMLEMIFTPRNSDR

SEQ ID NO: 171 - protein, 5 aa, Human immunodeficiency virus type 1
RIKQI

SEQ ID NO: 172 - protein, 11 aa, Human immunodeficiency virus type 1
RIKQIGMPGGK

SEQ ID NO: 173 - protein, 10 aa, Human immunodeficiency virus type 1
LLELDKWASL

SEQ ID NO: 174 - protein, 14 aa, Human immunodeficiency virus type 1
EQELLELDKWASLW

SEQ ID NO: 175 - protein, 33 aa, Human immunodeficiency virus type 1
VQQQNNLLRAIEAQQHLLQLTVWGIKQLQARIL

SEQ ID NO: 176 - protein, 16 aa, Human immunodeficiency virus type 1
HLLQLTVWGIKQLQAR

SEQ ID NO: 177 - protein, 36 aa, Human immunodeficiency virus type 1
YTHIIYSLIEQSQNQQEKNEQELLALDKWASLWNWF

SEQ ID NO: 178 - protein, 26 aa, Human immunodeficiency virus type 1
YTHIIYSLIEQSQNQQEKNEQELLEL

SEQ ID NO: 179 - protein, 19 aa, Homo sapiens
GRERRPRLSDRPQLPYLEA

SEQ ID NO: 180 - protein, 20 aa, Homo sapiens
REQRRFSVSTLRNLGLGKKS

SEQ ID NO: 181 - protein, 18 aa, Plasmodium yoelii
PNKLPRSTAVVHQLKRKH

SEQ ID NO: 182 - protein, 11 aa, Plasmodium yoelii
TAVVHQLKRKH

SEQ ID NO: 183 - protein, 22 aa, Plasmodium vivax
PAGDRADGQPAGDRAAAGQPAG

SEQ ID NO: 184 - protein, 12 aa, Avian leukosis virus
NQSWTMVSPINV

SEQ ID NO: 185 - protein, 16 aa, Avian leukosis virus
MIKNGTKRTAVTFGSV

SEQ ID NO: 186 - protein, 19 aa, Foot-and-mouth disease virus
PNLRGDLQVLAQKVARTLP

SEQ ID NO: 187 - protein, 26 aa, Foot-and-mouth disease virus
RYNRNAVPNLRGDLQVLAQKVARTLP

SEQ ID NO: 188 - protein, 17 aa, Hepatitis C virus
DAEFRHDSGYEVHHQKL

SEQ ID NO: 189 - protein, 34 aa, Hepatitis B virus
RRRGRSPRRRTPSPRRRRSQSPRRRRSQSRESQC

SEQ ID NO: 190 - protein, 16 aa, Hepatitis B virus
GIVNLEDPASRDLVVS

SEQ ID NO: 191 - protein, 17 aa, Hepatitis B virus
GIVNLEDPASRDLVVSC

SEQ ID NO: 192 - protein, 20 aa, Plasmodium falciparum
EYLNKIQNSLSTEWSPCSVT

SEQ ID NO: 193 - protein, 9 aa, Plasmodium vivax
  MOD_RES (4): Xaa at position 4 represents A or D
DRAXGQPAG

SEQ ID NO: 194 - protein, 9 aa, Plasmodium vivax
  MOD_RES (5): Xaa at position 5 represents G or D
ANGAXBQPG

SEQ ID NO: 195 - protein, 11 aa, Plasmodium vivax
APGANQEGGAA

SEQ ID NO: 196 - protein, 19 aa, Plasmodium vivax
YLDKVRATVGTEWTPCSVT

SEQ ID NO: 197 - protein, 21 aa, Plasmodium vivax
PAGDRADGQPAGDRAAGQPAG

SEQ ID NO: 198 - protein, 18 aa, Plasmodium vivax
DRAAGQPAGDRADGQPAG

SEQ ID NO: 199 - protein, 36 aa, Plasmodium vivax
ANGAGNQPGANGAGDQPGANGADNQPGANGADDQPG

SEQ ID NO: 200 - protein, 18 aa, Plasmodium vivax
ANGAGNQPGANGAGDQPG

SEQ ID NO: 201 - protein, 19 aa, Plasmodium vivax
QANGADNQPGANGADDQPG

SEQ ID NO: 202 - protein, 22 aa, Plasmodium vivax
APGANQEGGAAAPGANQEGGAA

SEQ ID NO: 203 - DNA, 24 bp, Artificial Sequence
  Description of Artificial Sequence Hepatitis B virus PCR primer with an NcoI restriction site
ttgggccatggacatcgaccctta

SEQ ID NO: 204 - DNA, 34 bp, Artificial Sequence
  Description of Artificial Sequence Hepatitis B virus PCR primer with an EcoRI restriction site.
gcggagctctttttccaaattaattaacacccac

SEQ ID NO: 205 - DNA, 30 bp, Artificial Sequence
  Description of Artificial Sequence Hepatitis B virus PCR primer with EcoRI and SacI restriction sites and an inserted lysine codon
cgcgagctcgatccagcgtctagagagacc

SEQ ID NO: 206 - DNA, 31 bp, Artificial Sequence; identical to SEQ ID NO: 70
  Description of Artificial Sequence Hepatitis B virus PCR primer with HindIII restriction site

SEQ ID NO: 207 - protein, 14 aa, Hepatitis B virus
CQEKQLDENANVQL

SEQ ID NO: 208 - protein, 13 aa, Hepatitis B virus
CSKKGPRASGNLI

SEQ ID NO: 209 - protein, 21 aa, Hepatitis B virus
CLLTEHRMTWDPAQPPRDLTE

SEQ ID NO: 210 - protein, 22 aa, Hepatitis B virus
CVKRMKESRLEDTQKHRVDFLQ

SEQ ID NO: 211 - protein, 6 aa, Artificial Sequence
  Description of Artificial Sequence Cytochrome P-450 fragment
CMQLRS

SEQ ID NO: 212 - protein, 6 aa, Artificial Sequence
  Description of Artificial Sequence Cytochrome P-450 fragment
CRFSIN

SEQ ID NO: 213 - protein, 5 aa, Artificial Sequence
  Description of Artificial Sequence Cytochrome P-450 fragment
CAVPR

SEQ ID NO: 214 - protein, 6 aa, Artificial Sequence
  Description of Artificial Sequence Cytochrome P-450 fragment
CVIPRS

SEQ ID NO: 215 - protein, 5 aa, Artificial Sequence
  Description of Artificial Sequence Cytochrome P-450 fragment
CFIPV

SEQ ID NO: 216 - protein, 6 aa, Artificial Sequence
  Description of Artificial Sequence Cytochrome P-450 fragment
CTVSGA

SEQ ID NO: 217 - protein, 6 aa, Artificial Sequence
  Description of Artificial Sequence Cytochrome P-450 fragment
CTLSGE

SEQ ID NO: 218 - protein, 20 aa, Hepatitis B virus
TWVGVNLEDPASRDLVVSYV

SEQ ID NO: 219 - DNA, 63 bp, Hepatitis B virus
gctacctgggtgggtgttaatttggaagatccagcgtctagagacctagtagtcagttatgtc

SEQ ID NO: 220 - protein, 21 aa, Artificial Sequence
  Description of Artificial Sequence K inserted at amino acid position 75 of Hepatitis B core
TWVGVKNLEDPASRDLVVSYV

SEQ ID NO: 221 - DNA, 41 bp, Artificial Sequence
  Description of Artificial Sequence Lysine codon aaa inserted to make HBc- K75 mutant
gctacctgggtgggtgttaaaaatttggaagatccagcgtc

SEQ ID NO: 222 - protein, 21 aa, Artificial Sequence
  Description of Artificial Sequence K inserted at amino acid position 76 of Hepatitis B core
TWVGVNKLEDPASRDLVVSYV

SEQ ID NO: 223 - DNA, 27 bp, Artificial Sequence
  Description of Artificial Sequence Lysine codon aaa inserted to make HBc-K76 mutant
ttaataaattggaagatccagcgtcta

SEQ ID NO: 224 - protein, 21 aa, Artificial Sequence
  Description of Artificial Sequence K inserted at position 77 of Hepatitis B virus core
TWVGVNLKEDPASRDLVVSYV

SEQ ID NO: 225 - DNA, 27 bp, Artificial Sequence
  Description of Artificial Sequence Lysine codon aaa inserted to make HBc-K77 mutant
ttaatttgaaagaagatccagcgtcta

SEQ ID NO: 226 - protein, 21 aa, Artificial Sequence
  Description of Artificial Sequence K inserted at amino acid position 78 of Hepatitis B core
TWVGVNLEKDPASRDLVVSYV

SEQ ID NO: 227 - DNA, 32 bp, Artificial Sequence
  Description of Artificial Sequence Lysine codon aaa inserted to make HBc-K78 mutant
ttaatttggaaaaagatccagcgtctagagac

SEQ ID NO: 228 - protein, 21 aa, Artificial Sequence
  Description of Artificial Sequence K inserted at amino acid position 79 fo Hepatitis B core.
TWVGVNLEDKPASRDLVVSYV

SEQ ID NO: 229 - DNA, 36 bp, Artificial Sequence
  Description of Artificial Sequence Lysine codon aaa inserted to make HBc-K79 mutant
ttaatttggaagataaaccagcgtctagagacctag

SEQ ID NO: 230 - protein, 21 aa, Artificial Sequence
  Description of Artificial Sequence K inserted at amino acid position 79 of Hepatitis B core
TWVGVNLEDPKASRDLVVSYV

SEQ ID NO: 231 - DNA, 39 bp, Artificial Sequence
  Description of Artificial Sequence Lysine codon aaa inserted to make HBc-K80 mutant
ttaatttggaagatccaaaagcgtctagagacctagtag

SEQ ID NO: 232 - protein, 21 aa, Artificial Sequence
  Description of Artificial Sequence K inserted at amino acid position 81 of Hepatitis B core
TWVGVNLEDPAKSRDLVVSYV

SEQ ID NO: 233 - DNA, 43 bp, Artificial Sequence
  Description of Artificial Sequence Lysine codon aaa inserted to make HBc-K81 mutant
ttaatttggaagatccagcgaaatctagagacctagtagtcag

SEQ ID NO: 234 - protein, 21 aa, Artificial Sequence
  Description of Artificial Sequence K inserted at amino acid position 82 of Hepatitis B core
TWVGVNLEDPASKRDLVVSYV

SEQ ID NO: 235 - DNA, 45 bp, Artificial Sequence
  Description of Artificial Sequence Lysine codon aaa inserted to make HBc-K82 mutant
ttaatttggaagatccagcgtctaaaagagacctagtagtcagtt

SEQ ID NO: 236 - protein, 21 aa, Artificial Sequence
  Description of Artificial Sequence K inserted at amino acid position 83 to Hepatitis B core
TWVGVNLEDPASRKDLVVSYV

SEQ ID NO: 237 - DNA, 50 bp, Artificial Sequence
  Description of Artificial Sequence Lysine codon aaa inserted to make HBc-K83 mutant
ttaatttggaagatccagcgtctagaaaagacctagtagtcagttatgtc

SEQ ID NO: 238 - protein, 21 aa, Artificial Sequence
  Description of Artificial Sequence K inserted at amino acid position 83 of Hepatitis B core
TWVGVNLEDPASRDKLVVSYV

SEQ ID NO: 239 - DNA, 50 bp, Artificial Sequence
  Description of Artificial Sequence Lysine codon aaa inserted to make HBc-K84 mutant
ttaatttggaagatccagcgtctagagacaaactagtagtcagttatgtc

SEQ ID NO: 240 - protein, 21 aa, Artificial Sequence
  Description of Artificial Sequence K inserted at amino acid position 85 of Hepatitis B core
TWVGVNLEDPASRDLKVVSYV

SEQ ID NO: 241 - DNA, 31 bp, Artificial Sequence
  Description of Artificial Sequence Lysine codon aaa inserted to make HBc-K85 mutant
ctcgagagacctaaaagtagtcagttatgtc

SEQ ID NO: 242 - protein, 36 aa, Hepatitis B virus
GIQWMEWDREINNYTSLIHSLIEESQNQQEKNEQEL

SEQ ID NO: 243 - DNA, 102 bp, Artificial Sequence
  Description of Artificial Sequence human cytochrome P450
aatttggatgtgggaagatcgtgagatcaacaattataccagcctgatacattctttaattgaagagtcccagaaccaacaggagaaaaatgaacaagagct

SEQ ID NO: 244 - DNA, 94 bp, Hepatitis B virus
cttgttcatttttctcctgttggttctgggactcttcaattaaagaatgtatcaggctggtataattgttgatctcacgatcttcccacatcca

SEQ ID NO: 245 - protein, 6 aa, Hepatitis B virus
MDIDPY

SEQ ID NO: 246 - protein, 217 aa, Spermophilus variegatus
MYLFHLCLVFACVPCPTVQASKLCLGWLWDMDIDPYKEFGSSYQLLNFLPLDFFPDLNALVDTAAALYEEELTGREHCSPHHTAIRQALVCWEELTRLITWMSENTTEEVRRIIVDHVNNTWGLKVRQTLWFHLSCLTFGQHTVQEFLVSFGVWIRTPAPYRPPNAPILSTLPEHTVIRRRGGSRAARSPRRRTPSPRRRRSQSPRRRRSQSPASNC

SEQ ID NO: 247 - protein, 183 aa, Hepatitis B virus
MDIDPYKEFGATVELLSFLPSDFFPSVRDLLDTASALYREALESPEHCSPHHTALRQAILCWGELMTLATWVGVNLEDPASRDLVVSYVNTNMGLKFRQLLWFHISCLTFGRETVIEYLVSFGVWIRTPPAYRPPNAPILSTLPETTVVRRRGRSPRRRTPSPRRRRSQSPRRRRSQSRESQC

SEQ ID NO: 248 - protein, 185 aa, Hepatitis B virus
MDIDPYKEFGATVELLSFLPSDFFPSVRDLLDTASALYREALESPEHCSPHHTALRQAILCWGELMTLATWVGNNLQDPASRDLVVNYVNTNMGLKIRQLLWFHISCLTFGRETVLEYLVSFGVWIRTPPAYRPPNAPILSTLPETTVVRRRDRGRSPRRRTPSPRRRRSQSPRRRRSQSRESQC

SEQ ID NO: 249 - protein, 185 aa, Hepatitis B virus
MDIDPYKEFGATVELLSFLPSDFFPSVRDLLDTASALYREALESPEHCSPHHTALRQAILCWGELMTLATWVGNNLEDPASRDLVVNYVNTNVGLKIRQLLWFHISCLTFGRETVLEYLVSFGVWIRTPPAYRPPNAPILSTLPETTVVRRRDRGRSPRRRTPSPRRRPSQSPRRRRSQSRESQC

SEQ ID NO: 250 - protein, 183 aa, Hepatitis B virus
MDIDPYKEFGATVELLSFLPSDFFPSVRDLLDTAAALYRDALESPEHCSPHHTALRQAILCWGDLMTLATWVGTNLEDPASRDLVVSYVNTNVGLKFRQLLWFHISCLTFGRETVLEYLVSFGVWIRTPPAYRPPNAPILSTLPETTVVRRRGRSPRRRTPSPRRRRSQSPRRRRSQSRESQC

SEQ ID NO: 251 - protein, 183 aa, Marmota monax
MDIDPYKEFGSSYQLLNFLPLDFFPDLNALVDTATALYEEELTGREHCSPHHTAIRQALVCWDELTKLIAWMSSNITSEQVRTIIVNHVNDTWGLKVRQSLWFHLSCLTFGQHTVQEFLVSFGVWIRTPAPYRPPNAPILSTLPEHTVIRRRGGARASRSPRRRTPSPRRRRSQSPRRRRSQC

SEQ ID NO: 252 - protein, 26 aa, Bos taurus
STPPLPWPWSPAALRLLQRPPEEPAA

SEQ ID NO: 253 - protein, 17 aa, Ebola virus
ATQVEQHHRRTDNDSTA

SEQ ID NO: 254 - protein, 17 aa, Ebola virus
HNTPVYKLDISEATQVE

SEQ ID NO: 255 - protein, 17 aa, Ebola virus
GKLGLITNTIAGVAVLI

SEQ ID NO: 256 - protein, 10 aa, Artificial Sequence
  Description of Artificial Sequenceflexible linker arm
GGGGSGGGGT

SEQ ID NO: 257 - protein, 9 aa, Artificial Sequence
  Description of Artificial Sequence  flexible linker arm
GGGGSGGGG

SEQ ID NO: 258 - DNA, 513 bp, Plasmodium falciparum
  CDS (1)..(507)
atggacatcgacccttataaagaatttggagctactgtggagttactctcgtttttgccttctgacttctttccttcagtacgagatcttctagataccgcctcagctctgtatcgggaagccttagagtctcctgagcattgttcacctcaccatactgcactcaggcaagcaattctttgctggggggaactaatgactctagctacctgggtgggtgttaatttggaagatggaattaacgctaatccgaacgctaatccgaacgctaatccgaacgctaatccggagctcccagcgtctagagacctagtagtcagttatgtcaacactaatatgggcctaaagttcaggcaactcttgtggtttcacatttcttgtctcacttttggaagagaaacagttatagagtatttggtgtctttcggagtgtggattcgcactcctccagcttatagaccaccaaatgcccctatcctatcaacacttccggagactactgttgtttagtaa

SEQ ID NO: 259 - protein, 169 aa, Plasmodium falciparum
MDIDPYKEFGATVELLSFLPSDFFPSVRDLLDTASALYREALESPEHCSPHHTALRQAILCWGELMTLATWVGVNLEDGINANPNANPNANPNANPELPASRDLVVSYVNTNMGLKFRQLLWFHISCLTFGRETVIEYLVSFGVWIRTPPAYRPPNAPILSTLPETTVV

SEQ ID NO: 260 - DNA, 513 bp, Plasmodium falciparum
  CDS (1)..(507)
atggacatcgacccttataaagaatttggagctactgtggagttactctcgtttttgccttctgacttctttccttcagtacgagatcttctagataccgcctcagctctgtatcgggaagccttagagtctcctgagcattgttcacctcaccatactgcactcaggcaagcaattctttgctggggggaactaatgactctagctacctgggtgggtgttaatttggaaggaattaacgctaatccgaacgctaatccgaacgctaatccgaacgctaatccggagctcgatccagcgtctagagacctagtagtcagttatgtcaacactaatatgggcctaaagttcaggcaactcttgtggtttcacatttcttgtctcacttttggaagagaaacagttatagagtatttggtgtctttcggagtgtggattcgcactcctccagcttatagaccaccaaatgcccctatcctatcaacacttccggagactactgttgtttagtaa

SEQ ID NO: 261 - protein, 169 aa, Plasmodium falciparum
MDIDPYKEFGATVELLSFLPSDFFPSVRDLLDTASALYREALESPEHCSPHHTALRQAILCWGELMTLATWVGVNLEGINANPNANPNANPNANPELDPASRDLVVSYVNTNMGLKFRQLLWFHISCLTFGRETVIEYLVSFGVWIRTPPAYRPPNAPILSTLPETTVV

SEQ ID NO: 262 - DNA, 519 bp, Plasmodium falciparum
  CDS (1)..(519)
atggacatcgacccttataaagaatttggagctactgtggagttactctcgtttttgccttctgacttctttccttcagtacgagatcttctagataccgcctcagctctgtatcgggaagccttagagtctcctgagcattgttcacctcaccatactgcactcaggcaagcaattctttgctggggggaactaatgactctagctacctgggtgggtgttaatttggaagatccagcgtctagagacctagtagtcagttatgtcaacactaatatgggcctaaagttcaggcaactcttgtggtttcacatttcttgtctcacttttggaagagaaacagttatagagtatttggtgtctttcggagtgtggattcgcactcctccagcttatagaccaccaaatgcccctatcctatcaacacttccggagactactgttgttggaattgaatatctgaacaaaatccagaactctctgtccaccgaatggtctccgtgctccgttacctagtaa

SEQ ID NO: 263 - protein, 171 aa, Plasmodium falciparum
MDIDPYKEFGATVELLSFLPSDFFPSVRDLLDTASALYREALESPEHCSPHHTALRQAILCWGELMTLATWVGVNLEDPASRDLVVSYVNTNMGLKFRQLLWFHISCLTFGRETVIEYLVSFGVWIRTPPAYRPPNAPILSTLPETTVVGIEYLNKIQNSLSTEWSPCSVT

SEQ ID NO: 264 - DNA, 516 bp, Plasmodium falciparum
  CDS (1)..(516)
atggacatcgacccttataaagaatttggagctactgtggagttactctcgtttttgccttctgacttctttccttcagtacgagatcttctagataccgcctcagctctgtatcgggaagccttagagtctcctgagcattgttcacctcaccatactgcactcaggcaagcaattctttgctggggggaactaatgactctagctacctgggtgggtgttaatttggaagatggaattaacgctaatccgaacgctaatccgaacgctaatccgaacgctaatccggagctcccagcgtctagagacctagtagtcagttatgtcaacactaatatgggcctaaagttcaggcaactcttgtggtttcacatttcttgtctcacttttggaagagaaacagttatagagtatttggtgtctttcggagtgtggattcgcactcctccagcttatagaccaccaaatgcccctatcctatcaacacttccggagactactgttgtttgctagtaa

SEQ ID NO: 265 - protein, 170 aa, Plasmodium falciparum
MDIDPYKEFGATVELLSFLPSDFFPSVRDLLDTASALYREALESPEHCSPHHTALRQAILCWGELMTLATWVGVNLEDGINANPNANPNANPNANPELPASRDLVVSYVNTNMGLKFRQLLWFHISCLTFGRETVIEYLVSFGVWIRTPPAYRPPNAPILSTLPETTVVC

SEQ ID NO: 266 - DNA, 579 bp, Plasmodium falciparum
  CDS (1)..(579)
atggacatcgacccttataaagaatttggagctactgtggagttactctcgtttttgccttctgacttctttccttcagtacgagatcttctagataccgcctcagctctgtatcgggaagccttagagtctcctgagcattgttcacctcaccatactgcactcaggcaagcaattctttgctggggggaactaatgactctagctacctgggtgggtgttaatttggaagatggaattaacgctaatccgaacgctaatccgaacgctaatccgaacgctaatccggagctcccagcgtctagagacctagtagtcagttatgtcaacactaatatgggcctaaagttcaggcaactcttgtggtttcacatttcttgtctcacttttggaagagaaacagttatagagtatttggtgtctttcggagtgtggattcgcactcctccagcttatagaccaccaaatgcccctatcctatcaacacttccggagactactgttgttggaattgaatatctgaacaaaatccagaactctctgtccaccgaatggtctccgtgctccgttacctagtaa

SEQ ID NO: 267 - protein, 191 aa, Plasmodium falciparum
MDIDPYKEFGATVELLSFLPSDFFPSVRDLLDTASALYREALESPEHCSPHHTALRQAILCWGELMTLATWVGVNLEDGINANPNANPNANPNANPELPASRDLVVSYVNTNMGLKFRQLLWFHISCLTFGRETVIEYLVSFGVWIRTPPAYRPPNAPILSTLPETTVVGIEYLNKIQNSLSTEWSPCSVT

SEQ ID NO: 268 - DNA, 591 bp, Plasmodium falciparum
  CDS (1)..(591)
atggacatcgacccttataaagaatttggagctactgtggagttactctcgtttttgccttctgacttctttccttcagtacgagatcttctagataccgcctcagctctgtatcgggaagccttagagtctcctgagcattgttcacctcaccatactgcactcaggcaagcaattctttgctggggggaactaatgactctagctacctgggtgggtgttaatttggaagatggaattaacgcgaatccgaacgtggatccgaatgccaaccctaacgccaacccaaatgcgaacccagagctcccagcgtctagagacctagtagtcagttatgtcaacactaatatgggcctaaagttcaggcaactcttgtggtttcacatttcttgtctcacttttggaagagaaacagttatagagtatttggtgtctttcggagtgtggattcgcactcctccagcttatagaccaccaaatgcccctatcctatcaacacttccggagactactgttgttggaattgaatatctgaacaaaatccagaactctctgtccaccgaatggtctccgtgctccgttacctagtaa

SEQ ID NO: 269 - protein, 195 aa, Plasmodium falciparum
MDIDPYKEFGATVELLSFLPSDFFPSVRDLLDTASALYREALESPEHCSPHHTALRQAILCWGELMTLATWVGVNLEDGINANPNVDPNANPNANPNANPELPASRDLVVSYVNTNMGLKFRQLLWFHISCLTFGRETVIEYLVSFGVWIRTPPAYRPPNAPILSTLPETTVVGIEYLNKIQNSLSTEWSPCSVT

SEQ ID NO: 270 - DNA, 561 bp, Human immunodeficiency virus type 1
  CDS (1)..(561)
atggacatcgacccttataaagaatttggagctactgtggagttactctcgtttttgccttctgacttctttccttcagtacgagatcttctagataccgcctcagctctgtatcgggaagccttagagtctcctgagcattgttcacctcaccatactgcactcaggcaagcaattctttgctggggggaactaatgactctagctacctgggtgggtgttaatttggaagatggaattcaatggatggaatgggatcgtgagatcaacaattataccagcctgatacattctttaattgaagagtcccagaaccaacaggagaaaaatgaacaagagctcccagcgtctagagacctagtagtcagttatgtcaacactaatatgggcctaaagttcaggcaactcttgtggtttcacatttcttgtctcacttttggaagagaaacagttatagagtatttggtgtctttcggagtgtggattcgcactcctccagcttatagaccaccaaatgcccctatcctatcaacacttccggagactactgttgtttagtaa

SEQ ID NO: 271 - protein, 185 aa, Human immunodeficiency virus type 1
MDIDPYKEFGATVELLSFLPSDFFPSVRDLLDTASALYREALESPEHCSPHHTALRQAILCWGELMTLATWVGVNLEDGIQWMEWDREINNYTSLIHSLIEESQNQQEKNEQELPASRDLVVSYVNTNMGLKFRQLLWFHISCLTFGRETVIEYLVSFGVWIRTPPAYRPPNAPILSTLPETTVV

SEQ ID NO: 272 - DNA, 564 bp, Human immunodeficiency virus type 1
  CDS (1)..(564)
atggacatcgacccttataaagaatttggagctactgtggagttactctcgtttttgccttctgacttctttccttcagtacgagatcttctagataccgcctcagctctgtatcgggaagccttagagtctcctgagcattgttcacctcaccatactgcactcaggcaagcaattctttgctggggggaactaatgactctagctacctgggtgggtgttaatttggaagatggaattcaatggatggaatgggatcgtgagatcaacaattataccagcctgatacattctttaattgaagagtcccagaaccaacaggagaaaaatgaacaagagctcccagcgtctagagacctagtagtcagttatgtcaacactaatatgggcctaaagttcaggcaactcttgtggtttcacatttcttgtctcacttttggaagagaaacagttatagagtatttggtgtctttcggagtgtggattcgcactcctccagcttatagaccaccaaatgcccctatcctatcaacacttccggagactactgttgtttgctagtaa

SEQ ID NO: 273 - protein, 186 aa, Human immunodeficiency virus type 1
MDIDPYKEFGATVELLSFLPSDFFPSVRDLLDTASALYREALESPEHCSPHHTALRQAILCWGELMTLATWVGVNLEDGIQWMEWDREINNYTSLIHSLIEESQNQQEKNEQELPASRDLVVSYVNTNMGLKFRQLLWFHISCLTFGRETVIEYLVSFGVWIRTPPAYRPPNAPILSTLPETTVVC

SEQ ID NO: 274 - DNA, 651 bp, Spermophilus variegatus
atgtatctttttcacctgtgccttgtttttgcctgtgttccatgtcctactgttcaagcctccaagctgtgccttggatggctttgggacatggacatagatccctataaagaatttggttcttcttatcagttgttgaattttcttcctttggacttttttcctgatctcaatgcattggtggacactgctgctgctctttatgaagaagaattaacaggtagggagcattgttctcctcatcatactgctattagacaggccttagtgtgttgggaagaattaactagattaattacatggatgagtgaaaatacaacagaagaagttagaagaattattgttgatcatgtcaataatacttggggacttaaagtaagacagactttatggtttcatttatcatgtcttacttttggacaacacacagttcaagaatttttggttagttttggagtatggattagaactccagctccttatagaccacctaatgcacccattttatcaactcttccggaacatacagtcattaggagaagaggaggttcaagagctgctaggtccccccgaagacgcactccctctcctcgcaggagaaggtctcaatcaccgcgtcgcagacgctctcaatctccagcttccaactgc

SEQ ID NO: 275 - DNA, 549 bp, Hepatitis B virus
atggacatcgacccttataaagaatttggagctactgtggagttactctcgtttttgccttctgacttctttccttcagtacgagatcttctagataccgcctcagctctgtatcgggaagccttagagtctcctgagcattgttcacctcaccatactgcactcaggcaagcaattctttgctggggggaactaatgactctagctacctgggtgggtgttaatttggaagatccagcgtctagagacctagtagtcagttatgtcaacactaatatgggcctaaagttcaggcaactcttgtggtttcacatttcttgtctcacttttggaagagaaacagttatagagtatttggtgtctttcggagtgtggattcgcactcctccagcttatagaccaccaaatgcccctatcctatcaacacttccggagactactgttgttagacgacgaggcaggtcccctagaagaagaactccctcgcctcgcagacgaaggtctcaatcgccgcgtcgcagaagatctcaatctcgggaatctcaatgt

SEQ ID NO: 276 - DNA, 555 bp, Hepatitis B virus
atggacattgacccttataaagaatttggagctactgtggagttactctcgtttttgccttctgacttctttccttccgtacgagatctcctagacaccgcctcagctctgtatcgagaagccttagagtctcctgagcattgctcacctcaccatactgcactcaggcaagccattctctgctggggggaattgatgactctagctacctgggtgggtaataatttgcaagatccagcatccagagatctagtagtcaattatgttaatactaacatgggtttaaagatcaggcaactattgtggtttcatatatcttgccttacttttggaagagagactgtacttgaatatttggtctctttcggagtgtggattcgcactcctccagcctatagaccaccaaatgcccctatcttatcaacacttccggaaactactgttgttagacgacgggaccgaggcaggtcccctagaagaagaactccctcgcctcgcagacgcagatctcaatcgccgcgtcgcagaagatctcaatctcgggaatctcaatgt

SEQ ID NO: 277 - DNA, 555 bp, Hepatitis B virus
atggacattgacccttataaagaatttggagctactgtggagttactctcgtttttgccttctgacttctttccttccgtcagagatctcctagacaccgcctcagctctgtatcgagaagccttagagtctcctgagcattgctcacctcaccatactgcactcaggcaagccattctctgctggggggaattgatgactctagctacctgggtgggtaataatttggaagatccagcatctagggatcttgtagtaaattatgttaatactaacgtgggtttaaagatcaggcaactattgtggtttcatatatcttgccttacttttggaagagagactgtacttgaatatttggtctctttcggagtgtggattcgcactcctccagcctatagaccaccaaatgcccctatcttatcaacacttccggaaactactgttgttagacgacgggaccgaggcaggtcccctagaagaagaactccctcgcctcgcagacgcagatctccatcgccgcgtcgcagaagatctcaatctcgggaatctcaatgt

SEQ ID NO: 278 - DNA, 549 bp, Hepatitis B virus
atggacattgacccttataaagaatttggagctactgtggagttactctcgtttttgccttctgacttctttccttccgtacgagatcttctagataccgccgcagctctgtatcgggatgccttagagtctcctgagcattgttcacctcaccatactgcactcaggcaagcaattctttgctggggagacttaatgactctagctacctgggtgggtactaatttagaagatccagcatctagggacctagtagtcagttatgtcaacactaatgtgggcctaaagttcagacaattattgtggtttcacatttcttgtctcacttttggaagagaaacggttctagagtatttggtgtcttttggagtgtggattcgcactcctccagcttatagaccaccaaatgcccctatcctatcaacgcttccggagactactgttgttagacgacgaggcaggtcccctagaagaagaactccctcgcctcgcagacgaagatctcaatcgccgcgtcgcagaagatctcaatctcgggaatctcaatgt

SEQ ID NO: 279 - DNA, 549 bp, Marmota monax
atggctttggggcatggacatagatccttataaagaatttggttcatcttatcagttgttgaattttcttcctttggacttctttcctgatcttaatgctttggtggacactgctactgccttgtatgaagaagaactaacaggtagggaacattgctctccgcaccatacagctattagacaagctttagtatgctgggatgaattaactaaattgatagcttggatgagctctaacataacttctgaacaagtaagaacaatcattgtaaatcatgtcaatgatacctggggacttaaggtgagacaaagtttatggtttcatttgtcatgtctcactttcggacaacatacagttcaagaatttttagtaagttttggagtatggatcaggactccagctccatatagacctcctaatgcacccattctctcgactcttccggaacatacagtcattaggagaagaggaggtgcaagagcttctaggtcccccagaagacgcactccctctcctcgcaggagaagatctcaatcaccgcgtcgcag

SEQ ID NO: 280 - protein, 13 aa, Artificial Sequence
  Description of Artificial Sequence human cytochrome P450
QEKQLDENANVQL

SEQ ID NO: 281 - protein, 7 aa, Artificial Sequence
  Description of Artificial Sequence modified portion of Hepatitis  B core
CVVTTEP

SEQ ID NO: 282 - DNA, 42 bp, Artificial Sequence
  Description of Artificial Sequencemodified portion of  Hepatitis B core
gcaagcttactattgaattccgcaaacaacagtagtctccgg

SEQ ID NO: 283 - protein, 26 aa, Artificial Sequence
  Description of Artificial Sequence  modified portion of Hepatitis B core
TTVVGIEYLNKIQNSLSTEWSPCSVT

SEQ ID NO: 284 - protein, 27 aa, Artificial Sequence
  Description of Artificial Sequence modified portion of Hepatitis B core
TTVVCGIEYLNKIQNSLSTEWSPASVT

SEQ ID NO: 285 - DNA, 51 bp, plasmid pKK223
ttcacacaggaaacagaattcccggggatccgtcgacctgcagccaagctt

SEQ ID NO: 286 - DNA, 38 bp, plasmid pKK223
ttcacataaggaggaaaaaacattgggatccgaagctt

SEQ ID NO: 287 - protein, 20 aa, Plasmodium yoelii
EFVKQISSQLTEEWSQCSVT

SEQ ID NO: 288 - protein, 14 aa, Escherichia coli
CCELCCYPACAGCN

SEQ ID NO: 289 - protein, 18 aa, Escherichia coli
NTFYCCELCCYPACAGCN

SEQ ID NO: 290 - protein, 18 aa, Escherichia coli
SSNYCCELCCYPACAGCN

SEQ ID NO: 291 - protein, 10 aa, Influenza virus
LIDALLGDPC

SEQ ID NO: 292 - protein, 9 aa, Influenza virus
TLIDALLGC

SEQ ID NO: 293 - protein, 42 aa, Homo sapiens [sequence omitted]

SEQ ID NO: 294 - protein, 11 aa, Homo sapiens
EDVGSNKGAII

SEQ ID NO: 295 - protein, 33 aa, Homo sapiens
DAEFRHDSGYEVHHQKLVFFAEDVGSNKGAIIG

SEQ ID NO: 296 - DNA, 60 bp, Homo sapiens
aattgatgcggaatttcgtcatgacagcggctatgaggtgcaccatcagaaactggagct

SEQ ID NO: 297 - DNA, 52 bp, Homo sapiens
ccagtttctgatggtgcacctcatagccgctgtcatgacgaaattccgcatc

SEQ ID NO: 298 - DNA, 42 bp, Homo sapiens
aattgaagatgtcggttctaacaagggggcaattatcgagct

SEQ ID NO: 299 - DNA, 34 bp, Homo sapiens
cgataattgcccccttgttagaaccgacatcttc

SEQ ID NO: 300 - DNA, 82 bp, Homo sapiens
gcgggaattgatgcggaatttcgtcatgacagcggctatgaggtgcaccatcagaaactggttttctttgccgaagatgtcg

SEQ ID NO: 301 - DNA, 83 bp, Homo sapiens
gcggagctccgctatgacaaccccacccaccattaagccgataattgcccccttgttagaaccgacatcttcggcaaagaaaa

SEQ ID NO: 302 - DNA, 53 bp, Homo sapiens
gcggagctcgataattgcccccttgttagaaccgacatcttcggcaaagaaaa

SEQ ID NO: 303 - DNA, 31 bp, Homo sapiens
gcgggaattctggatgcggaatttcgtcatg

SEQ ID NO: 304 - DNA, 17 bp, Homo sapiens
gcggagctccgctatga

SEQ ID NO: 305 - DNA, 31 bp, Homo sapiens; identical to SEQ ID NO: 303

SEQ ID NO: 306 - DNA, 18 bp, Homo sapiens
gcggagctcgataattgc

SEQ ID NO: 307 - protein, 24 aa, Haemophilus influenzae
MSLLTEVETPIRNEWGCRCNDSSD

SEQ ID NO: 308 - protein, 23 aa, Haemophilus influenzae
SLLTEVETPIRNEWGCRCNDSSD

SEQ ID NO: 309 - protein, 23 aa, Haemophilus influenzae
SLLTEVETPIRNEWGARANDSSD

SEQ ID NO: 310 - protein, 35 aa, Haemophilus influenzae
MGISLLTEVETPIRNEWGCRCNDSSDELLGWLWGI

SEQ ID NO: 311 - protein, 35 aa, Haemophilus influenzae; identical to SEQ ID NO: 310

SEQ ID NO: 312 - protein, 23 aa, Influenza A virus
SLLTEVETPIRNEWGARANDSSD

SEQ ID NO: 313 - protein, 19 aa, Influenza A virus
EQQSAVDADDSHFVSIELE

(19) United States
(12) Patent Application Publication
(10) Pub. No.: US 2004/0152876 A1
(43) Pub. Date: Aug. 5, 2004

(54) Immunogenic HBc chimer particles having enhanced stability

(76) Inventor: Ashley J. Birkett, Escondido, CA (US)

Correspondence Address: WELSH & KATZ, LTD, 120 S RIVERSIDE PLAZA, 22ND FLOOR, CHICAGO, IL, 60606, US

(21) Appl. No.: 10/806,006

(22) Filed: Mar. 22, 2004

Related U.S. Application Data
(62) Division of application No. 09/930,915, filed on Aug. 15, 2001.
(60) Provisional application No. 60/226,867, filed on Aug. 22, 2000.
(60) Provisional application No. 60/225,843, filed on Aug. 16, 2000.

(30) Foreign Application Priority Data
Aug. 16, 2001 (WO) PCT/US01/41759

(51) Int. Cl. C07K 14/005

(52) U.S. Cl. 530/350

(57) ABSTRACT

A chimeric, carboxy-terminal truncated hepatitis B virus nucleocapsid protein (HBc) is disclosed that is engineered for both enhanced stability of self-assembled particles and the display of an immunogenic epitope. The display of the immunogenic epitope is displayed in the immunogenic loop of HBc, whereas the enhanced stability of self-assembled particles is obtained by the presence of at least one heterologous cysteine residue near the carboxy-terminus of the chimer molecule. Methods of making and using the chimers are also disclosed.

### CROSS-REFERENCE TO RELATED APPLICATIONS

This a continuation-in-part of application Serial No. 60/225,843, filed Aug. 16, 2000, and application Serial No. 60/226,867, filed Aug. 22, 2000 whose disclosures are incorporated herein by reference.

### TECHNICAL FIELD

The present invention relates to the intersection of the fields of immunology and protein engineering, and particularly to a chimeric hepatitis B virus (HBV) nucleocapsid protein that is engineered for both enhanced stability of self-assembled particles and the display of an immunogenic epitope.

### BACKGROUND OF THE INVENTION

The family hepadnaviridae are enveloped DNA-containing animal viruses that can cause hepatitis B in humans (HBV). The hepadnavirus family includes hepatitis B viruses of other mammals, e.g., woodchuck (WHV), and ground squirrel (GSHV), and avian viruses found in ducks (DHV) and herons (HeHV). Hepatitis B virus (HBV) used herein refers to a member of the family hepadnaviridae, unless the discussion is referring to a specific example.

The nucleocapsid or core of the mammalian hepatitis B virus (HBV or hepadnavirus) contains a sequence of 183 or 185 amino acid residues, depending on viral subtype, whereas the duck virus capsid contains 262 amino acid residues. Hepatitis B core protein monomers of the several hepadnaviridae self-assemble in infected cells into stable aggregates known as hepatitis B core protein particles (HBc particles). Two three-dimensional structures are reported for HBc particles. A first that comprises a minor population contains 90 copies of the HBc subunit protein as dimers or 180 individual monomeric proteins, and a second, major population that contains 120 copies of the HBc subunit protein as dimers or 240 individual monomeric proteins. These particles are referred to as T=4 or T=3 particles, respectively, wherein “T” is the triangulation number. These HBc particles of the human-infecting virus (human virus) are about are about 30 or 34 nm in diameter, respectively. Pumpens et al. (1995) Intervirology, 38:63-74; and Metzger et al. (1998) J. Gen. Viol., 79:587-590.

Conway et al., (1997) Nature, 386:91-94, describe the structure of human HBc particles at 9 Angstrom resolution, as determined from cryo-electron micrographs. Bottcher et al. (1997), Nature, 386:88-91, describe the polypeptide folding for the human HBc monomers, and provide an approximate numbering scheme for the amino acid residues at which alpha-helical regions and their linking loop regions form. Zheng et al. (1992), J. Biol. Chem., 267(13):9422-9429 report that core particle formation is not dependent upon the arginine-rich C-terminal domain, the binding of nucleic acids or the formation of disulfide bonds based on their study of mutant proteins lacking one or more cysteines and others' work with C-terminal-truncated proteins [Birnbaum et al., (1990) J. Virol. 64, 3319-3330].

The hepatitis B nucleocapsid or viral core protein (HBc) has been disclosed as an immunogenic carrier moiety that stimulates the T cell response of an immunized host animal. See, for example, U.S. Pat. No. 4,818,527, No 4,882,145 and No. 5,143,726. A particularly useful application of this carrier is its ability to present foreign or heterologous B cell epitopes at the site of the immunodominant loop that is present at about residue positions 70-90, and more usually recited as about positions 75 through 85 from the amino-terminus (N-terminus) of the protein. Clarke et al. (1991) F. Brown et al. eds., Vaccines 91, Cold Spring Harbor Laboratory, Cold Spring Harbor, N.Y., pp.313-318.

During viral replication, HBV nucleocapsids associate with the viral RNA pre-genome, the viral reverse transcriptase (Pol), and the terminal protein (derived from Pol) to form replication competent cores. The association between the nucleocapsid and the viral RNA pre-genome is mediated by an arginine-rich domain at the carboxyl-terminus (C-terminus). When expressed in heterologous expression systems, such as E. coli where viral RNA pre-genome is absent, the protamine-like C-terminus; i.e., residues at positions 150 through 183, can bind E. coli RNA. Zhang et al. (1992) JBC, 267 (13) 9422-29.

In an application as a vaccine carrier moiety, it is preferable that the HBV nucleocapsids not bind nucleic acid derived from the host. Birnbaum et al. (1990) J. Virol., 64:3319-3330 showed that the protamine-like C-terminal domain of HBV nucleocapsids could be deleted without interfering with the protein's ability to assemble into virus-like particles. It is thus reported that proteins truncated to about position 144; i.e., containing the HBc sequence from position one through about 144, can self-assemble, whereas deletions beyond residue 139 abrogate capsid assembly [F. Birnbaum & M. Nassal (1990) J. Virl., 64: 3319-30].

Zlotnick et al., (1997) Proc. Natl. Acad. Sci., USA, 94:9556-9561 studied the assembly of full length and truncated HBc proteins in to particles. In addition to discussing full length molecules, those authors reported the preparation of a truncated protein that contained the HBc sequence from position 1 through 149 in which the cysteines at positions 48, 61 and 107 were each replaced by alanines and in which a cysteine residue was added at the C-terminus (position 150). That C-terminal mercaptan was used for linkage to a gold atom cluster for labeling in electron microscopy.

More recently, Metzger E T al. (1998) J. Gen. Viol., 79:587-590 reported that the proline at position 138 (Pro-138 or P138) of the human viral sequence is required for particle formation. Those authors also reported that assembly capability of particles truncated at the carboxy-terminus to lengths of 142 and 140 residues was affected, with assembly capability being completely lost with truncations resulting in lengths of 139 and 137 residues.

Several groups have shown that truncated particles exhibit reduced stability relative to standard hepatitis B core particles [Galena et al. (1989) J. Virol., 63:4645-4652; Inada, et al. (1989) Virus Res., 14:27-48], evident by variability in particle sizes and the presence of particle fragments in purified preparations [Maassen et al., (1994) Arch. Virol., 135:131-142]. Thus, prior to the report of Metzger et al., above, Pumpens et al., (1995) Intervirology, 38:63-74 summarized the literature reports by stating that the carboxy-terminal border for HBc sequences required for self-assembly was located between amino acid residues 139 and 144, and that the first two or three amino-terminal residues could be replaced by other sequences, but elimination of four or eleven amino-terminal residues resulted in the complete disappearance of chimeric protein in transformed E. coli cells. Neirynck et al., (October 1999) Nature Med., 5(10):1157-1163 reported that particle formation occurred on E. coli expression of a HBc chimer that contained the N-terminal 24-residue portion of the influenza M2 protein fused to HBc at residue 5.

Recombinantly-produced hybrid HBc particles bearing internal insertions (referred to in the art as HBc chimeric particles or HBc chimers) containing various inserted polypeptide sequences have been prepared by heterologous expression in a wide variety of organisms, including E. coli, B. subtilis, Vaccinia, Salmonella typhimurium, Saccharomyces cerevisiae. See, for example Pumpens et al. (1995) Intervirology, 38:63-74, and the citations therein that note the work of several research groups.

The above Pumpens et al. report lists particle-forming chimers in which the inserted polypeptide sequence is at the N-terminus, the C-terminus and between the termini. Insert lengths reported in that article are 24 to 50 residues at the N-terminus, 7 to 43 residues internally, and 11 to 741 residues at the C-terminus.

Kratz et al., (1999) Proc. Natl. Acad. Sci., U.S.A., 96:1915-1920 recently described the E. coli expression of chimeric HBc particles comprised of a truncated HBc sequence internally fused to the 238-residue green fluorescent protein (GFP). This chimer contained the inserted GFP sequence flanked by a pair of glycine-rich flexible linker arms replacing amino acid residues 79 and 80 of HBc. Those particles were said to effectively elicit antibodies against native GFP in rabbits as host animals.

U.S. Pat. No. 5,990,085 describes two fusion proteins formed from an antigenic bovine inhibin peptide fused into (i) the immunogenic loop between residues 78 and 79 and (ii) after residue 144 of carboxy-terminal truncated HBc. Expressed fusion proteins were said to induce the production of anti-inhibin antibodies when administered in a host animal. The titers thirty days after immunization reported in that patent are relatively low, being 1:3000-15,000 for the fusion protein with the loop insertion and 1:100-125 for the insertion after residue 144.

Chimeric hepatitis B core particles bearing internal insertions often appear to have a less ordered structure, when analyzed by electron microscopy, compared to particles that lack heterologous epitopes [Schodel et al. (1994) J. Exp. Med., 180:1037-1046]. In some cases, the insertion of heterologous epitopes into C-terminally truncated HBc particles has such a dramatic destabilizing affect that hybrid particles cannot be recovered following heterologous expression [Schodel et al. (1994) Infect. Immunol., 62:1669-1676]. Thus, many chimeric HBc particles are so unstable that they fall apart during purification to such an extent that they are unrecoverable or they show very poor stability characteristics, making them problematic for vaccine development.

A structural feature whereby the stability of full-length HBc particles could be retained, while abrogating the nucleic acid binding ability of full-length HBc particles, would be highly beneficial in vaccine development using the hepadnaviral nucleocapsid delivery system. Indeed, Ulrich et al. in their recent review of the use of HBc chimers as carriers for foreign epitopes [Adv. Virus Res., vol.50 (1998) Academic Press pages 141-182] note three potential problems to be solved for use of those chimers in human vaccines. A first potential problem is the inadvertent transfer of nucleic acids in a chimer vaccine to an immunized host. A second potential problem is interference from preexisting immunity to HBc. A third possible problem relates to the requirement of reproducible preparation of intact chimer particles that can also withstand long-term storage.

As disclosed hereinafter, the present invention provides one solution to the problems of HBc chimer stability as well as the substantial absence of nucleic acid binding ability of the construct, while providing powerfully immunogenic materials.

### BRIEF SUMMARY OF THE INVENTION

The present invention contemplates a recombinant hepadnavirus nucleocapsid protein; i.e., a hepatitis B core (HBc) chimeric protein [or chimer hepatitis B core protein molecule or HBc chimer molecule or just chimer] that self-assembles into particles after expression in a host cell. The chimeric protein (i) displays one or more immunogenic epitopes at the N-terminus, HBc immunogenic loop or C-terminus, or has a heterologous linker residue for a conjugated epitope in the immunogenic loop, and contains a cysteine residue at or near the C-terminus that confers enhanced stability to the particles. The chimeric protein is sufficiently free of arginine residues so that the self-assembled particles are substantially free of nucleic acid binding.

The present invention also contemplates an immunogenic particle comprised of recombinant hepatitis B core (HBc) chimeric protein molecules. The chimeric protein (i) displays one or more immunogenic epitopes at the N-terminus, HBc immunogenic loop or C-terminus, or (ii) has a heterologous linker residue for a conjugated epitope in the HBc immunogenic loop. That recombinant protein contains a cysteine residue at or near the C-terminus. The particles are substantially free of nucleic acid binding and exhibit enhanced stability relative to particles comprised of otherwise identical proteins that are free of the cysteine residue.

One embodiment of the invention contemplates a recombinant chimer hepatitis B core (HBc) protein molecule up to about 515 amino acid residues in length that

(a) contains (i) a sequence of at least about 130 of the N-terminal 150 amino acid residues of the HBc molecule including a covalently linked peptide-bonded heterologous epitope or a heterologous linker residue for a conjugated epitope present in the HBc immunodominant loop, or (ii) a sequence of at least about 135 residues of the N-terminal 150 HBC amino acid residues,

(b) contains one to ten, and more preferably, one to three cysteine residues toward the C-terminus of the molecule from the C-terminal residue of the HBc sequence present and within about 30 residues from the C-terminus of the chimer molecule [C-terminal cysteine residue(s)], and

(c) contains a sequence of at least five amino acid residues from HBc residue position 135 to the HBc C-terminus.

The contemplated chimer molecules (i) contain no more than 20 percent substituted amino acid residues in the HBc sequence, and (ii) self-assemble on expression in a host cell into particles that are substantially free of binding to nucleic acids. Those particles are substantially free of binding to nucleic acids and are more stable than are particles formed from an otherwise identical HBc chimer that lacks the above C-terminal cysteine residue(s) or where a C-terminal cysteine residue is present in the chimer and is replaced in the molecule by another residue such as an alanine residue.

In one aspect of this embodiment, a contemplated HBc chimer has a sequence of about 135 to about 515 amino acid residues and contains four serially peptide-linked domains that are denominated Domains I, II, III and IV. From the N-terminus, Domain I comprises about 71 to about 100 amino acid residues whose sequence includes at least the sequence of the residues of about position 5 through position 75 of HBc, and optionally includes a heterologous epitope containing up to about 30 amino acid residues peptide-bonded to one of HBc residues 1-4. Domain II comprises 5 to about 250 amino acid residues peptide-bonded to HBc residue 75 of Domain I in which (i) zero to all, and preferably at least 4, residues in a sequence of HBc positions 76 to 85 are present peptide-bonded to one to about 245 amino acid residues that are heterologous (foreign) to HBc and constitute a heterologous epitope such as a B cell epitope or a heterologous linker residue for an epitope such as a B cell epitope or (ii) the sequence of HBc at positions 76 to 85 is present free from heterologous residues. Domain III is an HBc sequence from position 86 through position 135 peptide-bonded to residue 85 of Domain II. Domain IV comprises (i) zero through fourteen residues of a HBc amino acid residue sequence from position 136 through 149 peptide-bonded to the residue of position 135 of Domain III, (ii) one to ten, and more preferably one to three, cysteine residues peptide-bonded C-terminal to that HBc sequence [C-terminal cysteine residue(s)] and (iii) zero to about 100, more preferably zero to about 50, and most preferably about 25 amino acid residues in a sequence heterologous to HBc from position 150 to the C-terminus, with the proviso that Domain IV contain at least 6 amino acid residues including the above one to ten cysteine residues of (ii).

A contemplated recombinant chimer protein forms particles that are substantially free of binding to nucleic acids and are more stable than are particles formed from a HBc chimer containing the same peptide-linked Domain I, II and III sequences and a Domain IV sequence that is otherwise same but lacks any cysteine residues or in which a cysteine residue is replaced by another residue such as an alanine residue. When chimer molecules are assembled into particles, those particles exhibit an absorbance ratio at 280 nm to 260 nm (280/260 absorbance ratio) of about 1.2 to about 1.7. The particles formed are believed to be of the T=4 structure, containing 240 monomeric HBc chimers or 120 dimer HBc chimers.

More broadly, a contemplated chimer particle comprises a C-terminal truncated HBc protein (to at least residue 149) that contains a heterologous epitope or a heterologous linker residue for an epitope in the immunodominant loop, or an uninterrupted immunodominant loop, and regardless of the amino acid residue sequence of the immunodominant loop, one to three C-terminal cysteine residues heterologous to the HBc sequence. Such a particle exhibits a 280/260 absorbance ratio of about 1.2 to about 1.7 and is more stable than a particle formed from an otherwise identical HBc chimer that lacks the above C-terminal cysteine residue(s) or where a single C-terminal cysteine residue is present in the chimer and is replaced by another residue.

Another embodiment comprises an inoculum or vaccine that comprises an above HBc chimer particle or a conjugate of a hapten with an above HBc chimer particle that is dissolved or dispersed in a pharmaceutically acceptable diluent composition that typically also contains water. When administered in an immunogenic effective amount to an animal such as a mammal or bird, an inoculum (i) induces antibodies that immunoreact specifically with the chimer particle or the conjugated (pendently-linked) hapten or (ii) activates T cells, or (iii) both. The antibodies so induced also preferably immunoreact specifically with (bind to) an antigen containing the hapten, such as a protein where the hapten is a peptide or a saccharide where the hapten is an oligosaccharide.

The present invention has several benefits and advantages.

One benefit of the invention is that chimer HBc particles are formed that are more stable on storage in aqueous compositions than are particles of similar sequence that lack any C-terminal cysteine residues.

An advantage of the invention is that chimer molecules are prepared that exhibit the self-assembly characteristics of native HBc particles, while not exhibiting the nucleic acid binding of those native particles.

Another benefit of the present invention is that chimer particles are formed that exhibit excellent B cell and T cell immunogenicities.

Another advantage is that chimer particles of the present invention are typically prepared in higher yield than are similar particles that are free of a C-terminal cysteine residue.

A further benefit of the invention is that chimer particles are formed that are often far more immunogenic than are similar conjugates that lack a C-terminal cysteine residue.

A further advantage is that immunogenicities of particles assembled from chimer molecules containing at least one C-terminal cysteine residue are enhanced as compared to similar particles assembled from chimer molecules lacking at least one C-terminal cyeteine residue.

Still further benefits and advantages will be apparent to the skilled worker form the disclosure that follows.

### BRIEF DESCRIPTION OF THE DRAWINGS

In the drawings forming a portion of this disclosure

FIG. 1 shows the modifications made to commercial plasmid vector pKK223-3 in the preparation of plasmid vector pKK223-3N used herein for preparation of some recombinant HBc chimers. The modified sequence (SEQ ID NO: 285) is shown below the sequence of the commercially available vector (SEQ ID NO: 286). The bases of the added NcoI site are shown in lower case letters with all of the added bases being shown with double underlines, whereas the deleted bases are shown as dashes. The two restriction sites present in this segment of the sequence (NcoI and HindIII) are indicated.

FIG. 2, shown in three panels as FIGS. 2A, 2B and 2C, schematically illustrates a preferred cloning strategy in which a malarial B cell epitope such as (NANP)₄ (SEQ ID NO:1) is cloned into the EcoRI and SacI sites of an engineered HBc gene (FIG. 2A) between positions 78 and 79, which destroys the EcoRI site, while preserving the SacI site. FIG. 2B shows DNA that encodes a T cell epitope such as that referred to as Pf/CS-UTC and a stop codon (SEQ ID NO:120) cloned into the EcoRI and HindIII sites at the C-terminus of an engineered, truncated HBc gene containing the first 149 HBc residues (HBc149). PCR amplification of the construct of FIG. 2B using a primer having a 5′-terminal SacI restriction site adjacent to a HBc-encoding sequence beginning at residue position 79 digestion of the amplified sequence and the construct of FIG. 2A with SacI, followed by ligation of the appropriate portions is shown in FIG. 2C to form a single gene construct referred to hereinafter as V12 that encodes B cell- and T cell-containing epitopes of an immunogen for a vaccine against P. falciparum.

FIG. 3 is a photograph of an SDS-PAGE analysis under reducing conditions to show the stabilizing effects on expressed particles of a codon for a single cysteine residue inserted in frame between the C-terminal codon (V149) and the termination codon of HBc in a chimer that also contains (NANP)₄ inserted between the amino acids of positions 78 and 79 (V2.Pf1+C), and a similar construct whose C-terminus is residue V149 (V2.Pf1) at day zero and after 15 days at 37° C. [Lane 1, V2.Pf1—day 0; Lane 2, V2.Pf1—day 15 at 37° C.; Lane 3, V2.Pf1+C, day 0; Lane 4, V2.Pf1+C—day 15 at 37° C.]

FIG. 4 is a photograph of an SDS-PAGE analysis under reducing conditions that illustrates the stabilizing effects on chimer HBc149 particles containing (NANP)₄ inserted between amino acids 78 and 79 and the cysteine-containing T cell epitope fused to the C-terminus [V2.Pf1+Pf/CS-UTC also referred to as V12.Pf1] as compared to a similar particle in which the C-terminal Cys was replaced by an Ala residue [V2.Pf1+Pf/CS-UTC(C17A) also referred to as V12.Pf1(C17A)] at day zero and after 28 days at 37° C. [Lane 1, V2.Pf1+Pf/CS-UTC—day zero; Lane 2, V2.Pf1+Pf/CS-UTC—day 28 at 37° C.; Lane 3, V2.Pf1+Pf/CS-UTC(C17A)—day zero; Lane 4, V2.Pf1+Pf/CS-UTC(C17A)—day 28 at 37° C.]

FIG. 5 is a graph showing the results of an indirect immunofluorescence assay (IFA) carried out using glutaraldehyde-fixed P. falciparum sporozoites and FITC-labeled anti-mouse IgG (gamma-chain specific) to detect bound antibody titers (log of 1/dilution; ordinate) over time in weeks (abscissa) for three chimeric immunogens after immunization in mice. Data for the prior art chimer immunogen, CS-2, are shown as squares, those for the recombinant HBc chimer V12.Pf1 are shown as diamonds, whereas those for the recombinant HBc chimer V12.Pf3.1 are shown as triangles.

FIG. 6 illustrates a reaction scheme (Scheme 1) that shows two reaction sequences for (I) forming an activated carrier for pendently linking a hapten to a chimeric hepatitis B core protein (sm-HBc) particle using sulpho-succinimidyl 4-(N-maleimidomethyl)cyclohexane 1-carboxylate (sulpho-SMCC), and then (II) linking a sulfhydryl-terminated (cysteine-terminated) hapten to the activated carrier to form a conjugate particle. The sm-HBc particle is depicted as a box having a single pendent amino group (for purposes of clarity of the figure), whereas the sulfhydryl-terminated hapten is depicted as a line terminated with an SH group.

FIG. 7, shown in two panels as FIG. 7A and FIG. 7B, provides an alignment of six published amino acid residue sequences for mammalian HBc proteins from six viruses. The first (SEQ ID NO:247), human viral sequence is of the ayw subtype and was published in Galibert et al. (1983) Nature, 281:646-650; the second human viral sequence (SEQ ID NO:248), of the adw subtype, was published by Ono et al. (1983) Nucleic Acids Res., 11(6): 1747-1757; the third human viral sequence (SEQ ID NO:249), is of the adw2 subtype and was published by Valenzuela et al., Animal Virus Genetics, Field et al. eds., Academic Press, New York (1980) pages 57-70; the fourth human viral sequence (SEQ ID NO:250), is of the adyw subtype that was published by Pasek et al. (1979) Nature, 282:575-579; the fifth sequence (SEQ ID NO:251), is that of the woodchuck virus that was published by Galibert et al. (1982) J. Virol., 41:51-65; and the sixth mammalian sequence, (SEQ ID NO:246), is that of the ground squirrel that was published by Seeger et al. (1984) J. Virol., 51:367-375.

FIG. 8 is a photograph of an SDS-PAGE analysis under reducing conditions following incubations at 37° C. for 0, 1 and 2 days that illustrates the stabilizing effects on (1) chimer HBc149 particles containing the P. falciparum (NANP)₄ immunogenic sequence inserted between HBc amino acid residues 78 and 79 that also contain a carboxy-terminal universal P. falciparum malarial T cell epitope peptide-bonded to HBc position 149 [UTC; V12.Pf1=V2.Pf1+Pf/CS-UTC], and (2) similar particles in which the cysteine at position 17 of the UTC was mutated to be an alanine residue and a cysteine residue was added at residue position 150, between the HBc residue at position 149 and the beginning of the UTC [V12.Pf1(C17A)+C150].

FIG. 9 is a photograph of an SDS-PAGE analysis under reducing conditions following particle preparation that shows the ICC-1438 monomer construct was unstable (Lane 2) as compared to the ICC-1492 construct (Lane 3), with HBc-149 (Lane 1), ICC-1475 (Lane 4) and ICC-1473 (Lane 5) serving as additional molecular weight controls.

### DEFINITIONS

Numerals utilized in conjunction with HBc chimers indicate the position in the HBc ayw amino acid residue sequence of SEQ ID NO: 247 at which one or more residues has been added to the sequence, regardless of whether additions or deletions to the amino acid residue sequence are present. Thus, HBc149 indicates that the chimer ends at residue 149, whereas HBc149+C150 indicates that that same chimer contains a cysteine residue at HBc position 150. On the other hand, the malarial CS protein universal T cell epitope (UTC) is 20 residues long, and a replacement of the cysteine at position 17 in that sequence by an alanine is referred to as CS-UTC(C17A).

The term “antibody” refers to a molecule that is a member of a family of glycosylated proteins called immunoglobulins, which can specifically bind to an antigen.

The word “antigen” has been used historically to designate an entity that is bound by an antibody or receptor, and also to designate the entity that induces the production of the antibody. More current usage limits the meaning of antigen to that entity bound by an antibody or receptor, whereas the word “immunogen” is used for the entity that induces antibody production or binds to the receptor. Where an entity discussed herein is both immunogenic and antigenic, reference to it as either an immunogen or antigen is typically made according to its intended utility.

“Antigenic determinant” refers to the actual structural portion of the antigen that is immunologically bound by an antibody combining site or T-cell receptor. The term is also used interchangeably with “epitope”. The words “antigenic determinant” and “epitope” are used somewhat more broadly herein to include additional residues that are heterologous to the HBc sequence but may not actually be bound by an antibody. Thus, for example, the malarial CS protein repeat sequences (NANP)₄ and NANPNVDP(NANP)₃NVDP of SEQ ID Nos:1 and 21 are each thought to contain more than one actual epitope, but are considered herein to each constitute a single epitope. Use of both of those sequences in a single HBc chimer molecule is considered to be a use of a plurality of epitopes.

The word “conjugate” as used herein refers to a hapten operatively linked to a carrier protein, as through an amino acid residue side chain of the carrier protein such as a lysine, aspartic or glutamic acid, tyrosine or cysteine residue.

The term “conservative substitution” as used herein denotes that one amino acid residue has been replaced by another, biologically similar residue. Examples of conservative substitutions include the substitution of one hydrophobic residue such as isoleucine, valine, leucine or methionine for another, or the substitution of one polar residue for another such as between arginine and lysine, between glutamic and aspartic acids or between glutamine and asparagine and the like.

The term “corresponds” in its various grammatical forms as used in relation to peptide sequences means the peptide sequence described plus or minus up to three amino acid residues at either or both of the amino- and carboxy-termini and containing only conservative substitutions in particular amino acid residues along the polypeptide sequence.

The term “Domain” is used herein to mean a portion of a recombinant HBc chimer molecule that is identified by (i) residue position numbering relative to the position numbers of HBcAg subtype ayw as reported by Galibert et al., (1979) Nature, 281:646-650 (SEQ ID NO:246). The polypeptide portions of at least chimer Domains I, II and III are believed to exist in a similar tertiary form to the corresponding sequences of naturally occurring HBcAg.

As used herein, the term “fusion protein” designates a polypeptide that contains at least two amino acid residue sequences not normally found linked together in nature that are operatively linked together end-to-end (head-to-tail) by a peptide bond between their respective carboxy- and amino-terminal amino acid residues. The fusion proteins of the present invention are HBc chimers that induce the production of antibodies that immunoreact with a polypeptide or pathogen-related immunogen that corresponds in amino acid residue sequence to the polypeptide or pathogen-related portion of the fusion protein.

The phrase “hepatitis B” as used here refers in its broadest context to any member of the family hepadnaviridae, as discussed before.

The term “residue” is used interchangeably with the phrase amino acid residue, and means a reacted amino acid as is present in a peptide or protein.

As used herein, the term “expression vector” means a DNA sequence that forms control elements that regulate expression of a structural gene that encodes a protein so that the protein is formed.

As used herein, the term “operatively linked” used in the context of a nucleic acid means that a gene is covalently bonded in correct reading frame to another DNA (or RNA as appropriate) segment, such as to an expression vector so that the structural gene is under the control of the expression vector. The term “operatively linked” used in the context of a protein, polypeptide or chimer means that the recited elements are covalently bonded to each other.

As used herein, the term “promoter” means a recognition site on a DNA sequence or group of DNA sequences that provide an expression control element for a gene and to which RNA polymerase specifically binds and initiates RNA synthesis (transcription) of that gene.

As used herein, the term “recombinant DNA molecule” means a hybrid DNA sequence comprising at least two nucleotide sequences not normally found together in nature.

As used herein, the term “vector” means a DNA molecule capable of replication in a cell and/or to which another DNA segment can be operatively linked so as to bring about replication of the attached segment. A plasmid is an exemplary vector.

All amino acid residues identified herein are in the natural L-configuration. In keeping with standard polypeptide nomenclature, J. Biol. Chem., 243:3557-59, (1969), abbreviations for amino acid residues are as shown in the following Table of Correspondence:

TABLE OF CORRESPONDENCE

| | SYMBOL | |
|---|---|---|
| 1-Letter | 3-Letter | AMINO ACID |
| Y | Try | L-tyrosine |
| G | Gly | glycine |
| F | Phe | L-phenylalanine |
| M | Met | L-methionine |
| A | Ala | L-alanine |
| S | Ser | L-serine |
| I | Ile | L-isoleucine |
| L | Leu | L-leucine |
| T | Thr | L-threonine |
| V | Val | L-valine |
| P | Pro | L-proline |
| K | Lys | L-lysine |
| H | His | L-histidine |
| Q | Gln | L-glutamine |
| E | Glu | L-glutamic acid |
| W | Trp | L-tryptophan |
| R | Arg | L-arginine |
| D | Asp | L-aspartic acid |
| N | Asn | L-asparagine |
| C | Cys | L-cysteine |

### DETAILED DESCRIPTION OF THE INVENTION

The present invention contemplates a chimeric hepadnavirus nucleocapsid protein; i.e., a recombinant hepatitis B core (HBc) protein, that is engineered to (a) display an immunogenic B cell or T cell epitope, a linker for attachment of an immunogenic B cell or T cell epitope or a truncated HBc protein, (b) exhibit enhanced stability when present in a self-assembled particle, as well as exhibit (c) a substantial absence of nucleic acid binding as a self-assembled particle. A contemplated HBc chimer is truncated at the C-terminus of the molecule relative to a native HBc molecule.

Thus, the chimeric protein displays one or more immunogenic epitopes at the N-terminus, in the HBc immunogenic loop or C-terminus, or a linker for such an epitope in the immunogenic loop. The chimeric protein contains a cysteine residue at or near the C-terminus that confers enhanced stability to the self-assembled particles. The chimeric protein is sufficiently free of arginine residues downstream of (toward the carboxy-terminus from) HBc residue position 149 so that the self-assembled particles are substantially free of nucleic acid binding.

For ease of discussion, contemplated chimer sequences and sequence position numbers referred to herein are based on the sequence and position numbering of the human hepatitis B core protein of subtype ayw [Galibert et al. (1979) Nature, 281:64:650]. It is to be understood, however, that in view of the great similarity between the mammalian hepadnavirus capsid protein sequences and similar particle formation exhibited by those proteins, which are well-known to skilled workers, a discussion regarding human HBc subtype ayw is also applicable to subtype adw, as well as the woodchuck and ground squirrel proteins. As a consequence of those great similarities, HBc sequences are recited generally herein as a “HBc” sequence, unless otherwise stated.

In one embodiment, a contemplated HBc chimer is up to about 515 residues in length and

(a) contains (i) a sequence of at least about 130 of the N-terminal 150 amino acid residues of the HBc molecule including a covalently linked heterologous epitope or a heterologous linker residue for a conjugated epitope present peptide-bonded in the HBc immunodominant loop, or (ii) a sequence of at least about 135 residues of the N-terminal 150 HBc amino acid residues,

(b) contains one to ten, and more preferably one to three, cysteine residues toward the C-terminus of the molecule from the C-terminal residue of the HBc sequence present and within about 30 residues from the C-terminus of the chimer molecule [C-terminal cysteine residue(s)], and

(c) contains a sequence of at least five amino acid residues from HBc residue position 135 to the HBc C-terminus. Five of those six residues are preferably of the HBc sequence from positions 136-140, with the sixth being the required cysteine.

The contemplated chimer self-assembles into particles when the chimer protein molecules are expressed in a host cell, and those particles are substantially free of binding to nucleic acids and are more stable (1) than are particles formed from an otherwise identical HBc chimer that lacks the above one to ten cysteine residues [C-terminal cysteine residue(s)] or (2) where a single C-terminal cysteine residue is present in the chimer and is replaced by another residue such as an alanine residue.

In one aspect, a preferred HBc chimer has a sequence of about 135 to about 515 L-α-amino acid residues and contains four serially peptide-linked domains; i.e., Domains I, II, III and IV. Those four domains are linked together in the same manner as are native proteins, as compared to polypeptides that contain residues of other than α-amino acids and therefore cannot form peptide bonds, those that contain D-amino acid residues, or oligopeptide conjugates in which two or more polypeptides are operatively linked through an amino acid residue side chain. A contemplated chimeric HBc protein can therefore be prepared by expression using the usual methods of recombinant technology.

From the amino-terminus, Domain I comprises about 71 to about 100 amino acid residues whose sequence includes at least the sequence of the residues of position 5 through position 75 of HBc. Preferably, the sequence of residues 1 through 75 of the HBc sequence is present as part of Domain I. Most preferably, Domain I is comprised only of the HBc sequence from position 1 through position 75.

Domain II comprises 5 to about 250 amino acid residues peptide-bonded to HBc residue 75 of Domain I of which (i) zero to all of the residues, and preferably at least 4 residues, and more preferably at least 8 residues, in a sequence of HBc at positions 76 through 85 are present peptide-bonded to one to about 245 residues that are heterologous (foreign) to HBc and constitute a heterologous linker residue for an epitope such as a B cell epitope or a heterologous epitope such as a B cell epitope itself or (ii) the sequence of HBc at positions 76 through 85 is present free from heterologous residues.

It is particularly preferred that the sequence of 10 residues of positions 76 through 85 (76-85 sequence) be present, but interrupted by one to about 245 residues of the heterologous linker or heterologous epitope. In other instances, it is particularly preferred that that 10 residue sequence be present alone, uninterrupted by any heterologous residue.

A chimer containing only HBc residues in this Domain together with the features discussed below is useful for inducing a B and/or T cell response to HBc itself. A preferred HBc chimer molecule with an uninterrupted 76-85 sequence contains the uninterrupted HBc amino acid residue sequence of position 1 through at least position 140, and more preferably contains the uninterrupted HBc amino acid residue sequence of position 1 through position 149, plus a single cysteine residue at the C-terminus, as discussed below.

Domain III is an HBc sequence from position 86 through position 135 peptide-bonded to residue 85.

Domain IV comprises (i) zero to fourteen residues of a HBc amino acid residue sequence from position 136 through 149 peptide-bonded to the residue of position 135 of Domain III, (ii) one to ten cysteine residues [C-terminal cysteine residue(s)], and (iii) zero to about 100 amino acid residues in a sequence heterologous to HBc from position 150 to the C-terminus that typically constitute one T cell epitope or a plurality of T cell epitopes, with the proviso that Domain IV contains at least a sequence of 6 amino acid residues from HBc residue position 135 to the C-terminus of the chimer, including the above one to ten cysteine residues of (ii). Preferably, Domain IV contains a sequence of zero to about 50 amino acid residues in a sequence heterologous to HBc, and more preferably that sequence is zero to about 25 residues.

In one aspect, a contemplated chimer molecule can thus be free of epitopes or residues heterologous to HBc, except for the C-terminal cysteine. In another aspect, a contemplated chimer molecule contains a heterologous epitope at the N-terminus peptide-bonded to one of HBc residues 1-5. In a further aspect, a contemplated chimer molecule contains a heterologous epitope or a heterologous linker residue for an epitope peptide-bonded near the middle of the molecule located between HBc residues 76 and 85 in the immunodominant loop. In a still further aspect, a heterologous epitope is located at the C-terminal portion of the chimer molecule peptide-bonded to one of HBc residues 136-149. In yet other aspects, two or three heterologous epitopes are present at the above locations, or one or two heterologous epitopes are present along with a heterologous linker residue for an epitope. Each of those chimer molecules also contains a C-terminal cysteine residue(s), as discussed before. Specific examples of several of these chimer molecules and their self-assembled particles are discussed hereinafter.

As already noted, a contemplated HBc chimer molecule can contain about 135 to about 515 amino acid residues. In preferred embodiments, HBc residues 1-5 are present, so that Domain I begins at HBc residue 1 and continues through residue 75; i.e., the HBc residue at HBc position 75. The heterologous epitope present in Domain II in the immunodominant loop preferably contains about 15 to about 50 residues, although an epitope as short as about 6 amino acid residues can induce and be recognized by antibodies and T cell receptors. Domain III contains HBc residues 86 through 135 peptide-bonded to residue 85. Domain IV contains a sequence of at least six residues that are comprised of (i) zero, one or a sequence of the residues of HBc positions 136 through 149 peptide-bonded to residue 135, (ii) at least one cysteine residue and (iii) optionally can contain a heterologous sequence of an epitope of up to about 100 residues, particularly when the HBc sequence ends at residue 135, although a shorter sequence of up to about 25 residues is more preferred.

In one embodiment, a particularly preferred chimer contains two heterologous epitopes. Those two heterologous epitopes are present in Domains I and II, or II and IV, or I and IV. One of the two heterologous epitopes is preferably a B cell epitope in some embodiments. In other embodiments, one of the two heterologous epitopes is a T cell epitope. More preferably, one of the two heterologous epitopes is a B cell epitope and the other is a T cell epitope. In addition, a plurality of B cell epitopes can be present at the B cell epitope location and a plurality of T cell epitopes can be present at the T cell epitope location.

In the embodiments in which the chimer molecule contains a heterologous epitope in Domain II, it is preferred that that epitope be one or more B cell epitopes, that the HBc sequence between amino acid residues 76 and 85 be present, but interrupted by the heterologous epitope(s), and that the chimer further include one or more T cell epitopes in Domain IV peptide-bonded to one of HBc residues 140-149.

This same preference holds for those chimer molecules in which the heterologous linker residue for a conjugated epitope is present in Domain II, thereby providing one or more heterologous epitopes in Domain II, with residues 76 and 85 present, but interrupted by the heterologous linker residue, with a T cell epitope being present peptide-bonded to one of HBc residues 140-149. The particles formed from such chimer molecules typically contain a ratio of conjugated epitope to C-terminal peptide-bonded T cell epitope of about 1:4 to 1:1, with a ratio of about 1:2 being common.

In an illustrative structure of an above-described chimer molecule, a heterologous linker residue for a conjugated epitope is present in Domain II and a T cell epitope is present in Domain IV, with no additional B cell epitope being present in Domain II. Such a chimer exhibits immunogenicity of the T cell epitope, while exhibiting minimal, if any, HBc antigenicity as measured by binding of anti-loop monoclonal antibodies in an ELISA assay as discussed hereinafter.

A preferred contemplated HBc chimer molecule contains a sequence of about 140 to about 515 residues. A preferred HBc chimer molecule containing two heterologous epitopes of preferred lengths of about 15 to about 50 residues each and a preferred HBc portion length of about 140 to about 149 residues has a sequence length of about 175 to about 240 amino acid residues. Particularly preferred chimer molecules continuing two heterologous epitopes have a length of about 190 to about 210 residues. It is to be understood that a wide range of chimer molecule lengths is contemplated in view of the variations in length of the N- and C-terminal HBc portions and differing lengths of the several contemplated epitopes that can be inserted in the immunogenic loop.

A contemplated recombinant protein, after expression in a host cell, self-assembles to form particles that are substantially free of binding to nucleic acids. The contemplated HBc chimer particles are generally spherical in shape and are usually homogeneous in size for a given preparation. These chimeric particles thus resemble native HBc particles that have a similar shape and size and can be recovered from infected persons.

A contemplated chimer particle comprises previously discussed chimer molecules. More broadly, such a chimer particle comprises a chimeric C-terminal truncated HBc protein that has a sequence of at least about 130 of the N-terminal 150 residues and contains (i) a heterologous epitope or a heterologous linker residue for an epitope in the immunodominant loop, or at least about 130 of the N-terminal 150 residues and an uninterrupted immunodominant loop and (ii) one to three C-terminal cysteine residues as previously described, and at least a 5 HBc residue sequence from position 135. Such a particle is sufficiently free of arginine residues so that the self-assembled particles are substantially free of nucleic acid binding and exhibits a 280/260 absorbance ratio of about 1.2 to about 1.7, as discussed herein after. Thus, a contemplated chimeric protein can be free of the HBc sequence between positions 150 and 183. A contemplated particle is more stable than a particle formed from an otherwise identical HBc chimer protein that lacks the above C-terminal cysteine residue(s). Similarly, a particle whose chimer molecule contains a single C-terminal cysteine residue is more stable than a particle in which that cysteine is replaced by another residue such as an alanine residue. In some instances, particles do not form unless a C-terminal cysteine is present. Examples of enhanced stabilities for both types of sequences are illustrated in the Examples that follow and is particularly evident in Examples relating to FIGS. 3, 4 and 8.

The substantial freedom of nucleic acid binding can be readily determined by a comparison of the absorbance of the particles in aqueous solution measured at both 280 and 260 nm; i.e., a 280/260 absorbance ratio. The contemplated particles do not bind substantially to nucleic acids that are oligomeric and/or polymeric DNA and RNA species originally present in the cells of the organism used to express the protein. Such nucleic acids exhibit an absorbance at 260 nm and relatively less absorbance at 280 nm, whereas a protein such as a contemplated chimer absorbs relatively less at 260 nm and has a greater absorbance at 280 nm.

Thus, recombinantly expressed HBc particles or chimeric HBc particles that contain the arginine-rich sequence at residue positions 150-183 (or 150-185) sometimes referred to in the art as the protamine region exhibit a ratio of absorbance at 280 nm to absorbance at 260 nm (280/260 absorbance ratio) of about 0.8, whereas particles sufficiently free of arginine residues so that the self-assembled particles are substantially free of nucleic acid binding such as particles that are free of the arginine-rich nucleic acid binding region of naturally occurring HBc like as those that contain fewer than three arginine or lysine residues or mixtures thereof adjacent to each other, or those having a native or chimeric sequence that ends at about HBc residue position 140 to position 149, exhibit a 280/260 absorbance ratio of about 1.2 to about 1.6.

Chimeric HBc particles of the present invention are substantially free of nucleic acid binding and exhibit a 280/260 absorbance ratio of about 1.2 to about 1.6, and more typically, about 1.4 to about 1.6. This range is due in large part to the number of aromatic amino acid residues present in Domains II and IV of a given chimeric HBc particle. That range is also in part due to the presence of the Cys in Domain IV of a contemplated chimer, whose presence can diminish the observed ratio by about 0.1 for a reason that is presently unknown.

The contemplated chimer HBc particles are more stable in aqueous buffer at 37° C. over a time period of about two weeks to about one month than are particles formed from a HBc chimer containing the same peptide-linked Domain I, II and III sequences and an otherwise same Domain IV sequence in which the one to ten cysteine residues [C-terminal cysteine residue(s)] are absent or a single C-terminal residue present is replaced by another residue such as an alanine residue. Stability of various chimer particles is determined as discussed hereinafter.

Thus, for example, particles containing a heterologous malarial epitope in Domain II [e.g. (NANP)₄] and a single cysteine residue C-terminal to residue valine 149 is more stable than otherwise identical particles assembled from chimer molecules whose C-terminal residue is valine 149. Similarly, particles containing the above malarial B cell epitope in Domain II and the universal malarial T cell epitope that contains a single cysteine near the C-terminus are more stable than are otherwise identical particles in which that cysteine is replaced by an alanine residue. See, FIGS. 3, 4 and 8 and the discussion relating thereto hereinafter.

A contemplated particle containing a C-terminal cysteine residue is also typically prepared in greater yield than is a particle assembled from a chimer molecule lacking a C-terminal cysteine. This increase in yield can be seen from the mass of particles obtained or from analytical gel filtration analysis using Superose® 6 HR as discussed hereinafter and shown in Table 17.

Domain I of a contemplated chimeric HBc protein constitutes an amino acid residue sequence of HBc beginning with at least amino acid residue position 5 through position 75, and Domain III constitutes a HBc sequence from position 86 through position 137. The sequences from any of the mammalian hepadnaviruses can be used for either of Domains I and III, and sequences from two or more viruses can be used in one chimer. Preferably, and for ease of construction, the human ayw sequence is used through out the chimer.

HBc chimers having a Domain I that contains more than a deletion of the first three amino-terminal (N-terminal) residues have been reported to result in the complete disappearance of HBc chimer protein in E. coli cells. Pumpens et al., (1995) Intervirology, 38:63-74. On the other hand, a recent study in which an immunogenic 23-mer polypeptide from the influenza M2 protein was fused to the HBc N-terminal sequence reported that the resultant fusion protein formed particles when residues 1-4 of the native HBc sequence were replaced. Neirynck et al. (October 1999) Nature Med., 5(10):1157-1163. Thus, the art teaches that particles can form when an added amino acid sequence is present peptide-bonded to one of residues 1-4 of HBc, whereas particles do not form if no additional sequence is present and more than residues 1-3 are deleted from the N-terminus of HBc.

An N-terminal sequence peptide-bonded to one of the first five N-terminal residues of HBc can contain a sequence of up to about 25 residues that are heterologous to HBc. Exemplary sequences include a B cell or T cell epitope such as those discussed hereinafter, the 23-mer polypeptide from the influenza M2 protein of Neirynck et al., above, a sequence of another (heterologous) protein such as galactosidase as can occur in fusion proteins as a result of the expression system used, or another hepatitis B-related sequence such as that from the Pre-S1 or Pre-S2 regions or the major HbsAg immunogenic sequence.

Domain II is a sequence of about 5 to about 250 amino acid residues. Of those residues, zero (none), and preferably at least 4 residues, and more preferably at least 8 residues, constitute portions of the HBc sequence at positions 76 to 85, and one to about 245 residues, and preferably one to about 50 residues are heterologous (foreign) to HBc. Those heterologous residues constitute (i) a heterologous linker residue for a epitope such as a B cell or T cell epitope or (ii) a heterologous B or T cell epitope that preferably contains 6 to about 50, more preferably about 15 to about 50, and most preferably about 20 to about 30 amino acid residues, and are positioned so that they are peptide-bonded between zero, or more preferably at least 4, to all of the residues of positions 76 through 85 of the HBc sequence. Heterologous B cell epitopes are preferably linked at this position by the linker residue or are peptide-bonded into the HBc sequence, and use of a B cell epitope is discussed illustratively hereinafter.

Those preferred at least 4 HBc residues can be all in one sequence such as residues 82-85, or can be split on either side of (flank) the heterologous residue(s) as where residues 76-77 and 84-85 are present or where residues 76 and 83-85 are present. More preferably, Domain II contains at least 8 residues of the HBc sequence from residue 76 to 85. Most preferably, the sequence of all 10 residues of positions 76 through 85 are present in the chimer.

The one to about 245 residues added to the HBc loop sequence is (are) heterologous to a HBc sequence. A single added heterologous residue is a heterologous linker residue for a B cell epitope as discussed before. The longer sequences, typically at least 6 amino acid residues long to about 50 amino acid residues long and more preferably about 15 to about 50 residues in length, as noted before, are in a sequence that comprises a heterologous immunogen such as a B cell epitope, except for heterologous residues encoded by restriction sites.

Exemplary peptide immunogens useful for both linkage to the linker residue after expression of a contemplated chimer and for expression within a HBc chimer are illustrated in Table A, below, along with the common name given to the gene from which the sequence is obtained, the literature or patent citation for published epitopes, and SEQ ID NO.

TABLE A
B Cell Epitopes

| | | | | |
|---|---|---|---|---|
| | | | | SEQ ID |
| Organism | Gene | Sequence | Citation* | NO |

| | | | | |
|---|---|---|---|---|
| Streptococcus | PspA | KLEELSDKIDELDAE | 1 | 3 |
| pneumoniae | QKKYDEDQKKTEE- | 1 | 4 | |
| | | KAALEKAASEEM- | | |
| | | DKAVAAVQQA | | |
| Cryptosporidium | P23 | QDKPADAPAAEAPA- | 2 | 5 |
| parvum | | AEPAAQQDKPADA | | |
| HIV | GP120 | RKRIHIGPGR- | 3 | 6 |
| | | AFYITKN | | |
| Foot-and-mouth | VP1 | YNGECRYNRNA- | 4 | 7 |
| virus | | VPNLRGDLQVL- | | |
| | | AQKVARTLP | | |
| Influenza Virus | HA | YRNLLWLTEK | 8 | 8 |
| A8/PR8 | | | | |
| A8/PR8/34 | M2 | SLLTEVETPIR- | 29 | 9 |
| | | NEWGCRCNGSSD | | |
| | | SLLTEVETPIR- | 29 | 10 |
| | | NEWGCRCNDSSD | | |
| | | SLLTEVETPIR- | | 312 |
| | | NEWGARANDSSD | | |
| | | EQQSAVDADDS- | 35 | 313 |
| | | HFVSIELE | | |
| Yersinia pestis | V Ag | DILKVIVDSMNHH- | 9 | 2.1 |
| | | GDARSKLREELAE- | | |
| | | LTAELKIYSVIQA- | | |
| | | EINKHLSSSGTIN- | | |
| | | IHDKSINLMDKNL- | | |
| | | YGYTDEEIFKASA- | | |
| | | EYKILEKMPQTTI- | | |
| | | QVDGSEKKIVSIK- | | |
| | | DFLGSENKRTGAL- | | |
| | | GNLKNSYSYNKDN- | | |
| | | NELSHFATTCSD | | |
| Haemophilus | pBOMP | CSSSNNDAA- | 10 | 12 |
| influenza | | GNGAAQFGGY | | |
| | | NKLGTVSYGEE | | 13 |
| | | NDEAAYSKN- | | 14 |
| | | RRAVLAY | | |
| Moraxella | copB | LDIEKDKKK- | 11 | 15 |
| catarrhalis | | RTDEQLQAE- | | |
| | | LDDKYAGKGY | | |
| | | LDIEKNKKK- | | 16 |
| | | RTEAELQAE- | | |
| | | LDDKYAGKGY | | |
| | | IDIEKKGKI- | | 17 |
| | | RTEAELLAE- | | |
| | | LNKDYPGQGY | | |
| Porphyromonas | HA | GVSPKVCKDVTV- | 12 | 18 |
| gingivalis | | EGSNEFAPVQNLT | | |
| | | RIQSTWRQKTV- | | 19 |
| | | DLPAGTKYV | | |
| Trypanosoma cruzi | | KAAIAPAKAAA- | 14 | 20 |
| | | APAKAATAPA | | |
| Plasmodium | CS | (NANP)₄ | 24 | 1 |
| falciparum | NANPNVDP- | | 21 | |
| | | (NANP)₃NVDP | | |
| | | NANPMVDP- | | 22 |
| | | (NANP)₃ | | |
| | | (NANP)₃NVDPNANP | | 23 |
| | | NANPNVDP- | | 24 |
| | | (NANP)₃NVDPNANP | | |
| | | NPNVDP(NANP)₃NV | | 25 |
| | | NPNVDP- | | 26 |
| | | (NANP)₃NVDP | | |
| | | NPNVDP(NANP)₃- | | 27 |
| | | NVDPNA | | |
| | | NVDP(NANP)₃NV | | 28 |
| | | NVDP(NANP)₃NVDP | | 29 |
| | | NVDP(NANP)₃- | | 30 |
| | | NVDPNA | | |
| | | DP(NANP)₃NV | | 31 |
| | | DP(NANP)₃NVDP | | 32 |
| | | DP(NANP)₃- | | 33 |
| | | NVDPNA | | |
| vivax | CS | GDRADGQPAG- | 20 | 34 |
| | | DRADGQPAG | | |
| | | RADDRAAGQP- | | 35 |
| | | AGDGQPAG | | |
| | | NGAGNQPG- | | 36 |
| | | ANGAGDQPG | | |
| | | ANGADNQPG- | 27 | 37 |
| | | ANGADDQPG | | |
| | | ANGAGNQPG- | | 38 |
| | | ANGADNQPG | | |
| | | ANGAGNQPG- | | 39 |
| | | ANGADDQPG | | |
| | | APGANQEGGAA- | 28 | 40 |
| | | APGANQEGGAA | | |
| | | ANGAGNQPGAN- | | 199 |
| | | GAGDQPGANGA- | | |
| | | DNQPGANGADD- | | |
| | | QPG | | |
| berghi | CS | DPPPPNPN- | 2 | 41 |
| | | DPPPPNPN | | |
| yoelli | CS | (QGPGAP)₄ | | 42 |
| Streptococcus | AgI/II | KPRPIYEA- | 16 | 43 |
| sobrinus | | KLAQNQK | | |
| | AKADYEAK- | | 44 | |
| | LAQYEKDL | | | |
| Shigella | Invasin | KDRTLIEQK | 18 | 45 |
| flexneri | | | | |
| Respiratory | syncitia | CSICSNNPT- | 19 | 46 |
| virus (RSV) | G | CWAICK | | |
| Entamoeba | lectin | VECASTVCQNDN- | 21 | 47 |
| histolytica | | SCPIIADVEKCNQ | | |
| Schistosoma | para | DLQSEISLSLE- | 22 | 48 |
| japonicum | | NGELIRRAKSA- | | |
| | | ESLLASELQRRVD | | |
| Schistosoma mansoni | para | DLQSEISLSLE- | 22 | 49 |
| | | NSELIRRAKAA- | | |
| | | ESLASDLQRRVD | | |
| Bovine Inhibin | α_{c} subunit | STPPLPWPW- | 30 | 252 |
| | | SPAALRLLQ- | | |
| | | RPPEEPAA | | |
| Ebola Virus | membrane- | ATQVEQHHRR- | 31 | 253 |
| | anchored | TDNDSTA | | |
| | glycoprotein | HNTPVYKLD- | 31 | 254 |
| | | ISEATQVE | | |
| | | GKLGLITNTI- | 31 | 255 |
| | | AGVAVLI | | |
| Escherichia coli | ST | CCELCCYPACAGCN | 33 | 288 |
| | | NTFYCCELCC- | 33 | 289 |
| | | YPACAGCN | | |
| | | SSNYCCELCC- | 33 | 290 |
| | | YPACAGCN | | |
| Alzheimer's disease | β-Amyloid | DAEFRHDSGYE- | 34 | 293 |
| | | VHHQKLVFFAE- | | |
| | | DVGSNKGAIIG- | | |
| | | LMVGGVVIA | | |
| | | DAEFRHDSGYE- | | 188 |
| | | VHHQKL | | |
| | | EDVGSNKGAII | | 294 |
| | | DAEFRHDSGYE- | | 295 |
| | | VHHQKLVFFAE- | | |
| | | DVGSNKGAIIG | | |

*Citations to published epitopes are provided following Table B.

The remaining residues of Domain II that are present on either side of the heterologous residue or sequence are the residues of HBc position 76 to position 85. Thus, in a typical example, where residues 78 through 82 have been replaced, the chimer sequence in Domain II is 76 through 77, followed by restriction site-encoded residues, the heterologous immunogenic (epitope) sequence, further restriction site-encoded residues, and then HBc sequence 84 through 85. A typical exemplary sequence of a chimer prepared by an insertion strategy between residues 78 and 79 is that of HBc from position 1 through 78, followed by restriction site-encoded residues, the heterologous immunogenic sequence, further restriction site-encoded residues and HBc sequence 79 through 85. The sequence of other contemplated chimers through Domains I and II should be apparent from these illustrations and those that follow and need not be enumerated.

As already noted, a heterologous linker for a conjugated epitope is peptide-bonded at a position in the HBc sequence between amino acid residues 76 and 85. As was the case for the heterologous epitope, the HBc sequence of residues 76 through 85 is preferably present, but interrupted by the heterologous linker for a conjugated epitope. This chimer preferably includes the HBc sequence of position 1 through at least position 140, plus a cysteine residue at the C-terminus of the chimer protein. More preferably, the HBc sequence of positions 1 through 149 are present, but interrupted between residues 76 and 85 by the heterologous linker for a conjugated epitope, and the chimer molecule contains a C-terminal cysteine. The heterologous linker for a conjugated epitope is most preferably a lysine (K) residue. Glutamic or aspartic acid, tyrosine and cysteine residues can also be used as linker residues, as can tyrosine and cysteine residues. It is noted that more than one linker can be present such as a sequence of three lysines, but such use is not preferred because heterogeneous conjugates can be formed from such use in which the conjugated hapten is bonded to one linker in a first chimer and to a different linker in a second chimer molecule. Published application PCT/US99/03055 discloses HBc chimer molecules containing one or more linking residues, but lacking a stabilizing C-terminal cysteine residue.

It is also noted that a heterologous epitope sequence present in a contemplated HBc chimer can also be separated from the HBc sequence residues by a “flexible linker arm” on one or both sides of (flanking) the heterologous immunogenic (epitope) sequence. This is particularly the case where the heterologous immunogenic sequence is greater than about 30 amino acid residues long. Exemplary flexible linker arm sequences typically contain about 4 to about 10 glycine residues that are thought to permit the inserted sequence to “bulge” outwardly from the otherwise bulging loop sequence and add further stability to the construct. Illustrative flexible linker arm sequences are disclosed in Kratz et al. (March 1999) Proc. Natl. Acad. Sci., U.S.A., 96:1915-1920 and are exemplified by the amino acid residue sequences:

| | |
|---|---|
| GGGGSGGGGT | SEQ ID NO:256 |
| GGGGSGGGG | SEQ ID NO:257 |

As was noted previously, Domain III constitutes the sequence of HBc from position 86 through position 135. Consequently, the sequence of the illustrative chimers discussed above for Domains I and II, can be extended so that the first-discussed chimer has the sequence of HBc from position 84 through position 135, and the second-discussed chimer has the sequence of HBc from position 79 through position 135.

Domain IV is a sequence that (i) optionally includes a HBc sequence from position 136 through 149, (ii) contains at least one cysteine residue, up to three cysteine residues, and (iii) up to about 100 amino acid residues in a sequence heterologous to HBc at position 150 to the C-terminus, with the proviso that Domain IV contain at least 6 amino acid residues, including the above one to ten cysteine residues of (ii). The Domain IV sequence heterologous to HBc more preferably contains up to about 50 amino acid residues, and most preferably contains up to about 25 residues. The Domain IV sequence can thus be substantially any cysteine-containing sequence, except the C-terminal HBc sequence from position 150 to the C-terminus.

The length of the Domain IV sequence can be six residues; i.e., a cysteine plus any five residues containing up to a total of three cysteines, to about 100 amino acid residues, with the length being sufficient so that a contemplated chimeric protein has a total length of about 135 to about 515 residues, and more preferably up to about 460 residues, and most preferably up to about 435 amino acid residues. Where an epitope is peptide-bonded to Domains I or II contains up to about 30 or about 50 residues, respectively, as is preferred for those epitopes, more preferred lengths of the chimer molecule, including the Domain IV epitope, are about 175 to about 240 residues. Particularly preferred chimer molecules containing two heterologous epitopes have a length of about 190 to about 210 residues. Freedom of the resulting particle from nucleic acid-binding is determined by determination of the 280/260 absorbance ratio as discussed previously.

The Domain IV sequence includes at least one cysteine (Cys) residue and can contain up to three Cys residues. It is preferred that the one or more Cys residues be at or within about five amino acid residues of the C-terminus of the chimeric protein molecule. In addition, when more than one Cys residue is present in a Domain IV sequence, it is preferred that those Cys residues be adjacent to each other.

It is also preferred that the Domain IV sequence constitute a T cell epitope, a plurality of T cell epitopes that are the same or different or an additional B cell epitope for the organism against which a contemplated chimer is intended to be used as an immunogen. Exemplary Domain IV T cell epitope sequences are provided in Table B, below, as in Table A.

TABLE B
T Cell Epitopes

| | | | | |
|---|---|---|---|---|
| | | | | SEQ |
| | | | Cita- | ID |
| Organism | Gene | Sequence* | tion | NO |

| | | | | |
|---|---|---|---|---|
| HIV | P24 | GPKEPFRDY- | 3 | 50 |
| | | VDRFYKC | | |
| Corynebacterium | toxin | FQVVHNSYN- | 5 | 51 |
| diptheriae | | RPAYSPGC | | |
| Borrelia | ospA | VEIKEGTVTLKRE- | 6 | 52 |
| burgdorferi | | IDKNGKVTVSLC | | |
| | | TLSKNISKSG- | 7 | 53 |
| | | EVSVELNDC | | |
| Influenza Virus | HA | SSVSSFERFEC | 8 | 54 |
| A8/PR8 | | LIDALLGDPC | 32 | 291 |
| | | TLIDALLGC | 32 | 292 |
| Trypanosoma | | SHNFTLVASVII- | 13 | 55 |
| cruzi | | EEAPSGNTC | | |
| Plasmodium | MSP1 | SVQIPKVPYPNGIVYC | 15 | 56 |
| falciparum | | DFNHYYTLKTGLEADC | | 57 |
| | | PSDKHIEQYKKI- | 23 | |
| | | KNSISC | | 58 |
| | | EYLNKIQNSLST- | 26 | |
| | | EWSPCSVT | | 59 |
| P. vivax | | YLDKVRATVGTE- | | 60 |
| | | WTPCSVT | | |
| P. yoelii | | EFVKQISSQLTE- | | 287 |
| | | EWSQCSVT | | |
| Streptococcus | AgI/Il | KPRPIYEAKL- | 16 | 61 |
| sobrinus | | AQNQKC | | |
| | | AKADYEAKLA- | | 62 |
| | | QYEKDLC | | |
| LCMV | NP | RPQASGVYM- | 17 | 63 |
| (lymphocytic | | GNLTAQC | | |
| choriomeningitis | | | | |
| virus) | | | | |
| Clostridium | tox | QYIKANSKFIG- | 20 | 64 |
| tetani | | ITELC | | |

*Underlined C (C) is not from the native sequence.

### CITATIONS

1. EPO 786 521A.

2. WO 98/07320.

3. U.S. Pat. No. 5,639,854.

4. U.S. Pat. No. 4,544,500.

5. EPO 399001 B1.

6. Bockenstedt et al. (1996) J. Immunol., 157, 12:5496.

7. Zhong et al. (1996) Eur. J. Immunol., 26, 11:2749.

8. Brumeanu et al. (1996) Immunotechnology, 2, 2:85.

9. Hill et al. (1997) Infect. Immun., 65, 11:4476.

10. EPO 432 220 B1.

11. WO 98/06851.

12. Kelly et al. (1997) Clin. Exp. Immunol., 110, 2:285.

13. Kahn et al. (1997) J. Immunol., 159, 9:4444.

14. WO 97/18475.

15. Ohta et al. (1997) Int. Arch. Allergy Immunol., 114, 1:15.

16. Staffileno et al. (1990) Arch. Oral Biol., 35: Suppl. 47S.

17. Saron et al. (1997) Proc. Natl. Acad. Sci. USA, 94, 7:3314.

18. Corthesy et al. (1996) J. Biol. Chem., 271, 52:33670.

19. Bastien et al. (1997) Virol., 234, 1:118.

20. Yang et al. (1997) Vaccine, 15, 4:377.

21. Lotter et al. (1997) J. Exp. Med., 185, 10:1793.

22. Nara et al. (1997) Vaccine 15, 1:79.

23. U.S. Pat. No. 4,886,782.

24. Zavala et al. (1985) Science, 228:1436.

25. Schodel et al. (1994) J. Exper. Med., 180:1037.

26. Calvo-Calleet al. (1997) J. Immunol. 159, 3:1362.

27. Qari et al. (1992) Mol. Biochem. Parasitol., 55(1-2):105.

28. Qari et al. (1993) Lancet, 341(8848):780.

29. Neirynck et al. (October 1999) Nature Med., 5(10):1157-1163.

30. Thompson et al. (1994) Eur. J. Biochem., 226(3):751-764.

31. Wilson et al. (2000) Science, 287:1664-1666.

32. Brown et al. (1993) J. Virol., 67(5):2887-2893.

33. U.S. Pat. No. 4,886,663.

34. Schenk et al. (Jul. 8, 1999) Nature, 400(6740):116-117.

35. Slepushkin et al. (1995) Vaccine, 13(15):1399-1402.

In addition to the at least one cysteine residue present in Domain IV, the amino acid sequence of HBc from residue position 1 through at least position 140 is preferably present in a contemplated chimer molecule and particle. The sequence from position 1 through position 149 is more preferably present. A B cell epitope is preferably present between residues 76 and 85 and at least a single cysteine residue or a T cell epitope containing a cysteine residue is present as a C-terminal addition to the HBc sequence. A contemplated recombinant HBc chimer is substantially free of bound nucleic acid. A contemplated chimer particle that contains an added Cys residue at or near the C-terminus of the molecule is also more stable at 37° C. than is a similar particle that does not contain that added Cys. This enhanced stability is illustrated in FIGS. 3, 4 and 8, and is discussed hereinafter.

A contemplated recombinant HBc chimer molecule is typically present and is used as a self-assembled particle. These particles are comprised of 180 to 240 chimer molecules (90 or 120 dimer pairs), usually 240 chimer molecules, that separate into protein molecules in the presence of disulfide reducing agents such as 2-mercaptoethanol, and the individual molecules are therefore thought to be bound together into the particle primarily by disulfide bonds.

Although not wishing to be bound by theory, it is believed that the observed enhanced stability and in some cases enhanced expression for a contemplated HBc chimer is due to the formation of a further cystine disulfide bond between proteins of the chimer particles. Regardless of whether present as a cysteine or a cystine, the C-terminal cysteine(s) residue is referred to as a cysteine inasmuch as that is the residue coded-for by the codon present in the nucleic acid from which the protein and assembled particle is expressed.

These particles are similar to the particles observed in patients infected with HBV, but these particles are non-infectious. Upon expression in various prokaryotic and eukaryotic hosts, the individual recombinant HBc chimer molecules assemble in the host into particles that can be readily harvested from the host cells, and purified, if desired.

As noted before, the HBc immunodominant loop is usually recited as being located at about positions 75 through 85 from the amino-terminus (N-terminus) of the intact protein. The heterologous B cell epitope-containing sequence of Domain II is placed into that immunodominant loop sequence. That placement substantially eliminates the HBc immunogenicity of the HBc loop sequence, while presenting the heterologous sequence or linker residue in an extremely immunogenic position in the assembled chimer particles.

In addition to the before-discussed N- and C-truncations, insertion of various epitopes and spacers, a contemplated chimer molecule can also contain conservative substitutions in the amino acid residues that constitute HBc Domains I, II, III and IV. Conservative substitutions are as defined before.

More rarely, a “nonconservative” change, e.g., replacement of a glycine with a tryptophan is contemplated. Analogous minor variations can also include amino acid deletions or insertions, or both. Guidance in determining which amino acid residues can be substituted, inserted, or deleted without abolishing biological activity or particle formation can be found using computer programs well known in the art, for example LASERGENE software (DNASTAR Inc., Madison, Wis.)

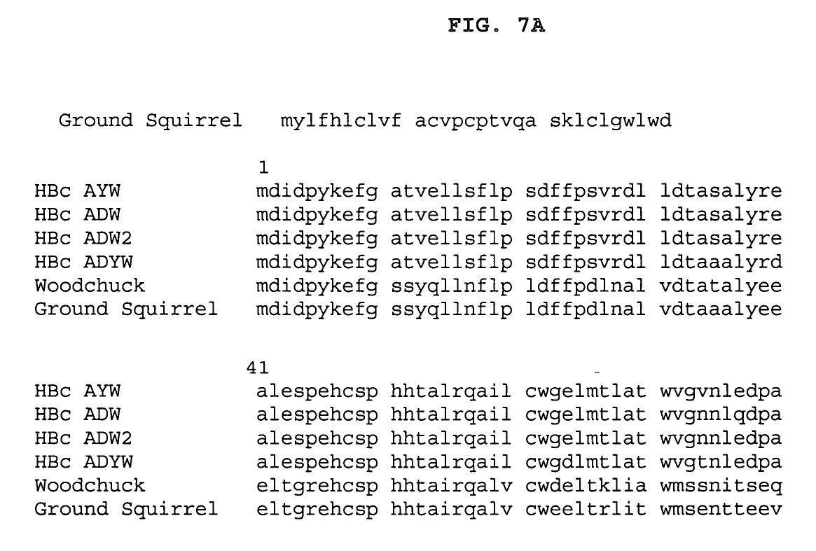

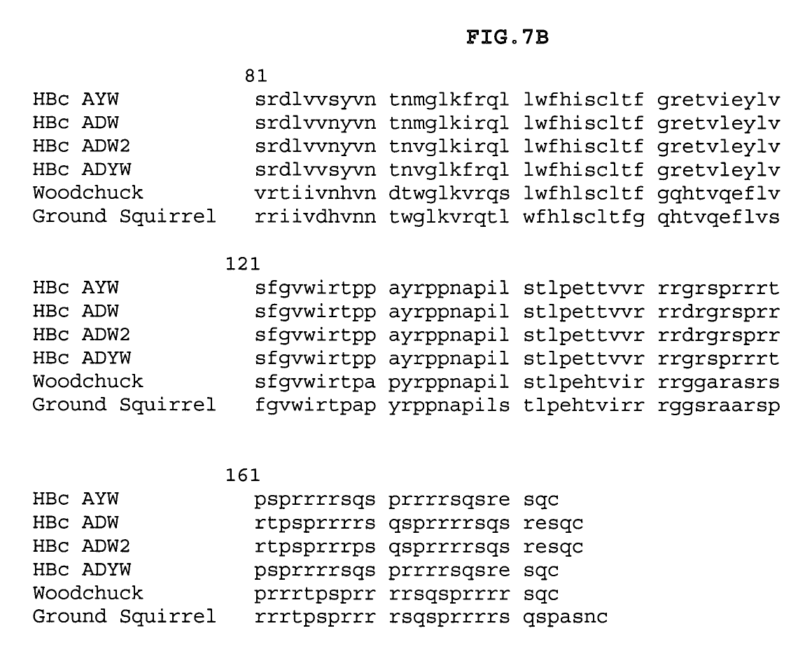

The HBc portion of a chimer molecule of the present invention; i.e., the portion having the HBc sequence that has other than a sequence or residue of an added epitope, linker, flexible linker arm or heterologous residue(s) that are a restriction enzyme artifact, most preferably has the amino acid residue sequence at positions 1 through 149 of subtype ayw that is shown in FIG. 7 (SEQ ID NO:247), less any portion or portions of the subtype ayw sequence that are absent because of truncation at one or both termini. Somewhat less preferred are the corresponding amino acid residue sequences of subtypes adw, adw2 and adyw that are also shown in FIG. 7 (SEQ ID NOs:248, 249 and 250). Less preferred still are the sequences of woodchuck and ground squirrel at aligned positions 1 through 149 that are the last two sequences of FIG. 7 (SEQ ID NOs:251 and 246). As noted elsewhere, portions of different sequences from different mammalian HBc proteins can be used together in a single chimer.

When the HBc portion of a chimer molecule of the present invention as above described has other than a sequence of a mammalian HBc molecule corresponding to positions 1 through 149, no more than about 20 percent of the amino acid residues are substituted as compared to SEQ ID NO:247 from position 1 through 149. It is preferred that no more than about 10 percent, and more preferably no more than about 5 percent, and most preferably no more than about 3 percent of the amino acid residues are substituted as compared to SEQ ID NO:247 from position 1 through 149.

A contemplated chimer of 149 HBc residues can therefore contain up to about 30 residues that are different from those of SEQ ID NO:247 at positions 1 through 149, and preferably about 15 residues. More preferably, about 7 or 8 residues are different from the ayw sequence (SEQ ID NO:247) at residue positions 1-149, and most preferably about 4 or 5 residues are different. Substitutions, other than in the immunodominant loop of Domain II or at the termini, are preferably in the non-helical portions of the chimer molecule and are typically between residues 1 to about 15 and residues 24 to about 50 to help assure particle formation. See, Koschel et al., J. Virol., 73(3):2153-2160 (March 1999).

Where a HBc sequence is truncated at the C-terminus beyond position 149 or at the N-terminus, or contains one or more deletions in the immunogenic loop, the number of substituted residues is proportionally different because the total length of the sequence is less that 149 residues. Deletions elsewhere in the molecule are considered conservative substitutions for purposes of calculation.

Chimer Preparation

A contemplated chimeric HBc immunogen is typically prepared using the well-known techniques of recombinant DNA technology. Thus, sequences of nucleic acid that encode particular polypeptide sequences are added to and deleted from the precursor sequence that encodes HBc to form a nucleic acid that encodes a contemplated chimer.

Either of two strategies is preferred for placing the heterologous epitope sequence into the loop sequence. The first strategy is referred to as replacement in which DNA that codes for a portion of the immunodominant loop is excised and replaced with DNA that encodes a heterologous epitope such as a B cell sequence. The second strategy is referred to as insertion in which a heterologous epitope is inserted between adjacent residues in the loop.

Site-directed mutagenesis using the polymerase chain reaction (PCR) is used in one exemplary replacement approach to provide a chimeric HBc DNA sequence that encodes a pair of different restriction sites, e.g. EcoRI and SacI, one near each end of the immunodominant loop-encoding DNA. Exemplary residues replaced are 76 through 81. The loop-encoding section is excised, a desired sequence that encodes the heterologous B cell epitope is ligated into the restriction sites and the resulting DNA is used to express the HBc chimer. See, for example, Table 2 of Pumpens et al., (1995) Intervirology, 38:63-74 for exemplary uses of this technique.

Alternatively, a single restriction site can be encoded into the region by site-directed mutagenesis, the DNA cut with a restriction enzyme to provide “sticky” ends, the sticky ends made blunt with endonuclease and a blunt-ended heterologous DNA segment ligated into the cut region. Examples of this type of sequence replacement into HBc can be found in the work reported in Schodel et al., (1991) F. Brown et al. eds., Vaccines 91, Cold Spring Harbor Laboratory, Cold Spring Harbor, N.Y., pp.319-325; Schodel et al., Behring Inst. Mitt., 1997(98): p. 114-119 and Schodel et al., J. Exp. Med., (1994) 180(3): p. 1037-4, the latter two papers discussing the preparation of vaccines against P. yoelii and P. berghei, respectively.

It has been found that the insertion position within the HBc immunogenic loop and the presence of loop residues can be of import to the activity of the immunogen. Thus, as is illustrated hereinafter, placement of a malarial B cell epitope between HBc residue positions 78 and 79 provides a particulate immunogen that is ten to one thousand times more immunogenic than placement of the same immunogen in an excised and replaced region between residues 76 and 81. In addition, placement of the same malarial immunogen between residues 78 and 79 as compared to between residues 77 and 78 provided an unexpected enhancement in immunogenicity of about 15-fold.

Insertion is therefore generally preferred. In an illustrative example of the insertion strategy, site-directed mutagenesis is used to create two restriction sites adjacent to each other and between codons encoding adjacent amino acid residues, such as those at residue positions 78 and 79. This technique adds twelve base pairs that encode four amino acid residues (two for each restriction site) between formerly adjacent residues in the HBc loop.

Upon cleavage with the restriction enzymes, ligation of the DNA coding for the heterologous B cell epitope sequence and expression of the DNA to form HBc chimers, the HBc loop amino acid sequence is seen to be interrupted on its N-terminal side by the two residues encoded by the 5′ restriction site, followed toward the C-terminus by the heterologous B-cell epitope sequence, followed by two more heterologous, non-loop residues encoded by the 3′ restriction site and then the rest of the loop sequence. This same strategy can be used for insertion into Domain I of a N-terminal sequence as was reported in Neirynck et al., (October 1999) Nature Med., 5(10):1157-1163 or for insertion into Domain IV of a T cell epitope or one or more cysteine residues that are not a part of a T cell epitope. A similar strategy using an insertion between residues 82 and 83 is reported in Schodel et al., (1990) F. Brown et al. eds., Vaccines 90, Cold Spring Harbor Laboratory, Cold Spring Harbor, N.Y., pp.193-198.

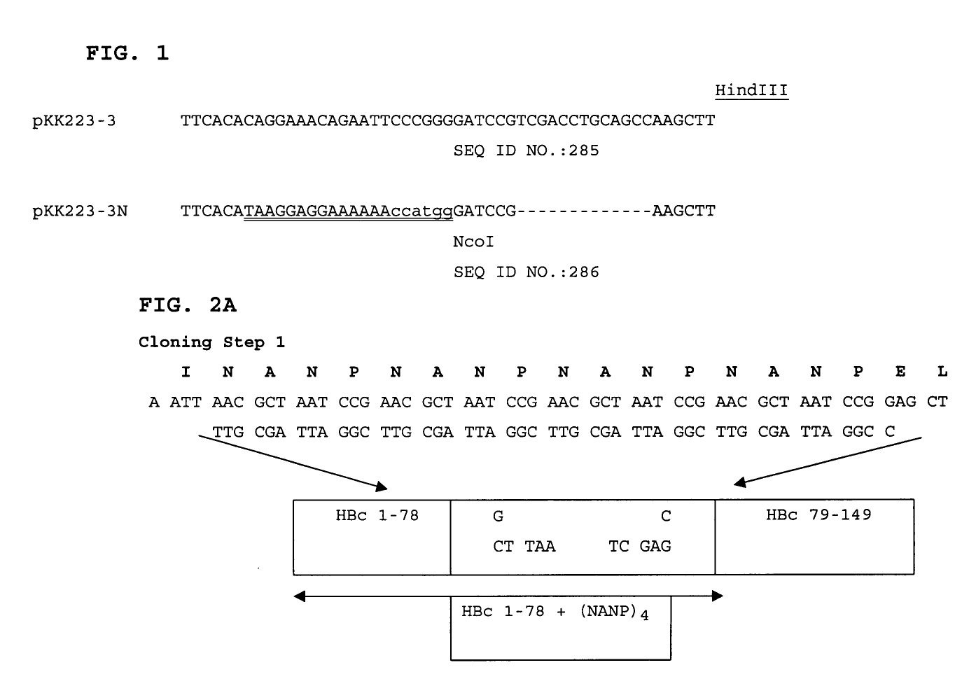

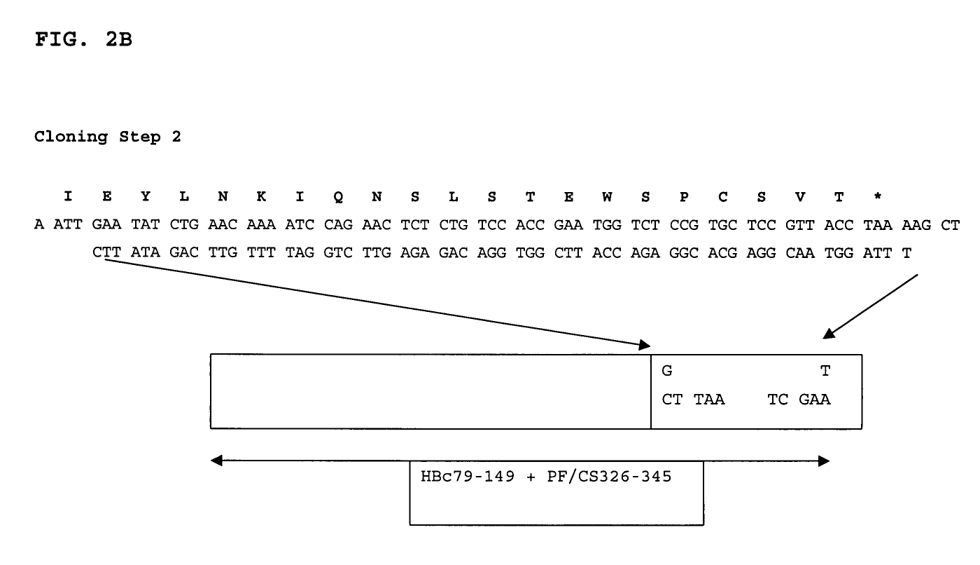

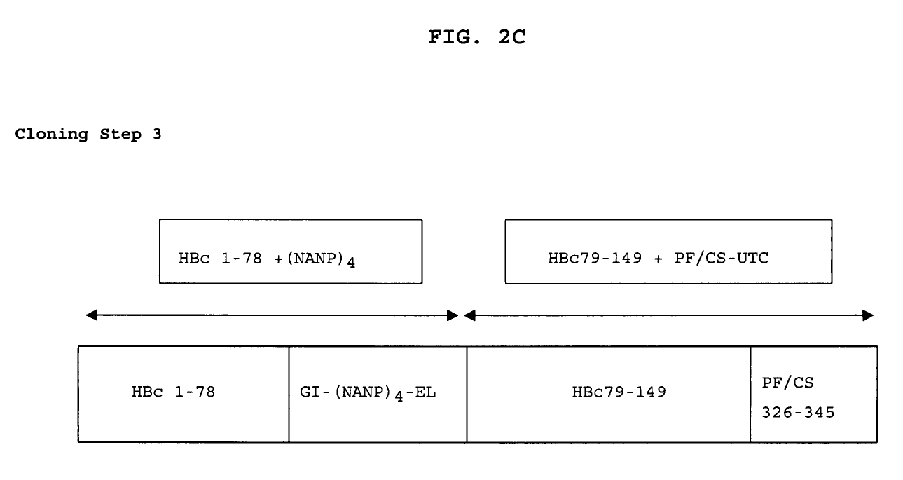

More specifically, this cloning strategy is illustrated schematically in FIGS. 2A, 2B and 2C. In FIG. 2A, a DNA sequence that encodes a C-terminal truncated HBc sequence (HBc149) is engineered to contain adjacent EcoRI and SacI sites between residues 78 and 79. Cleavage of that DNA with both enzymes provides one fragment that encodes HBc positions 1-78 3′-terminated with an EcoRI sticky end, whereas the other fragment has a 5′-terminal SacI sticky end and encodes residues of positions 79-149. Ligation of a synthetic nucleic acid having a 5′ AATT overhang followed by a sequence that encodes a desired malarial B cell epitope and a AGCT 3′overhang provides a HBc chimer sequence that encodes that B cell epitope flanked on each side by two heterologous residues [GlyIle (GI) and GluLeu (EL), respectively] between residues 78 and 79, while usually destroying the EcoRI site and preserving the SacI site.

A similar strategy is shown in FIG. 2B for insertion of a cysteine-containing sequence in Domain IV, such as a particularly preferred malarial T cell epitope that contains the P. falciparum CS protein sequence from position 326 through position 345 and is referred to herein as PF/CS326-345 (Pf-UTC). Here, EcoRI and HindIII restriction sites were engineered into the HBc DNA sequence after amino acid residue position 149. After digestion with EcoRI and HindIII, a synthetic DNA having the above AATT 5′overhang followed by a T cell epitope-encoding sequence, one or more stop codons and a 3′ AGCT overhang were ligated into the digested sequence to form a sequence that encoded HBc residues 1-149 followed by two heterologous residues (GI), the stop codon and the HindIII site.

PCR amplification using a forward primer having a SacI restriction site followed by a sequence encoding HBc beginning at residue position 79, followed by digestion with SacI and HindIII provided a sequence encoding HBc positions 79-149 plus the two added residues and the T cell epitope at the C-terminus. Digestion of the construct of FIG. 2B with SacI and ligation provided the complete gene encoding a desired recombinant HBc chimer immunogen having the sequence, from the N-terminus, of HBc positions 1-78, two added residues, the malarial B cell epitope, two added residues, HBc positions 79-149, two added residues, and the T cell epitope that is shown in FIG. 2C.

Similar techniques can be used to place a heterologous linker residue for conjugation of a B cell epitope into the loop region sequence. Contemplated linker residues include lysine (Lys), which is particularly preferred, aspartic acid (Asp), glutamic acid (Glu), cysteine (Cys) and tyrosine (Tyr).

It is noted that the amino acid residue sequence shown in SEQ ID NO: 247 contains a Glu and an Asp residue at positions 77 and 78. Nonetheless, introduction of an additional, heterologous, carboxyl-containing residue is still contemplated. The chemical reactivity of the existing glutamic and aspartic acids may be reduced by other factors. For example, it is known in the art that a neighboring proline, such as that found at position 79, can neutralize and thereby reduce the chemical reactivity of a proximal carboxyl group.

Here, using the first noted insertion strategy, five heterologous residues are placed into the loop sequence; one that is the heterologous linker residue for conjugating a B cell epitope and two residues adjacent on either side of that one residue that are themselves also adjacent to loop sequence residues and are an expression product of the inserted restriction sites (restriction enzyme artifacts). It is noted that one can also use site-directed mutagenesis to add a single codon into the HBc loop sequence that encodes the heterologous linker residue for a B cell epitope.

It is noted that the preferred use of two heterologous residues on either side of (flanking) a B cell or T cell epitope is a matter of convenience. As a consequence, one can also use zero to three or more added residues that are not part of the HBc sequence on either or both sides of an inserted sequence. One or both ends of the insert and HBc nucleic acid can be “chewed back” with an appropriate nuclease (e.g. S1 nuclease) to provide blunt ends that can be ligated together. Added heterologous residues that are neither part of the inserted B cell or T cell epitopes nor a part of the HBc sequence are not counted in the number of residues present in a recited Domain.

It is also noted that one can also synthesize all or a part of a desired recombinant HBc chimer nucleic acid using well-known synthetic methods as is discussed and illustrated in U.S. Pat. No. 5,656,472 for the synthesis of the 177 base pair DNA that encodes the 59 residue ribulose bis-phosphate carboxylase-oxygenase signal peptide of Nicotiana tabacum. For example, one can synthesize Domains I and II with a blunt or a “sticky end” that can be ligated to Domains III and IV to provide a construct that expresses a contemplated HBc chimer that contains zero added residues to the N-terminal side of the B cell epitope and zero to three added residues on the C-terminal side or at the Domain II/III junction or at some other desired location.

An alternative insertion technique was reported in Clarke et al. (1991) F. Brown et al. eds., Vaccines 91, Cold Spring Harbor Laboratory, Cold Spring Harbor, N.Y., pp.313-318. Here, taking advantage of the degeneracy of the genetic code, those workers engineered a single restriction site corresponding to residues 80 and 81 that encoded the original residues present at those positions. Their expressed HBc chimers thereby contained no restriction site-encoded residues, and contained the residues of the HBc loop immediately adjacent to the inserted sequence.

A nucleic acid sequence (segment) that encodes a previously described HBc chimer molecule or a complement of that coding sequence is also contemplated herein. Such a nucleic acid segment is present in isolated and purified form in some preferred embodiments.

In living organisms, the amino acid residue sequence of a protein or polypeptide is directly related via the genetic code to the deoxyribonucleic acid (DNA) sequence of the gene that codes for the protein. Thus, through the well-known degeneracy of the genetic code additional DNAs and corresponding RNA sequences (nucleic acids) can be prepared as desired that encode the same chimer amino acid residue sequences, but are sufficiently different from a before-discussed gene sequence that the two sequences do not hybridize at high stringency, but do hybridize at moderate stringency.

High stringency conditions can be defined as comprising hybridization at a temperature of about 50°-55° C. in 6×SSC and a final wash at a temperature of 68° C. in 1-3×SSC. Moderate stringency conditions comprise hybridization at a temperature of about 50° C. to about 65° C. in 0.2 to 0.3 M NaCl, followed by washing at about 50° C. to about 55° C. in 0.2×SSC, 0.1% SDS (sodium dodecyl sulfate).

A nucleic sequence (DNA sequence or an RNA sequence) that (1) itself encodes, or its complement encodes, a chimer molecule whose HBc portion from residue position 1 through 136, when present, is that of SEQ ID NOs: 246, 247, 248, 249, 250 or 251 and (2) hybridizes with a DNA sequence of SEQ ID NOs: 274, 275, 276, 277, 278 or 279 at least at moderate stringency (discussed above); and (3) whose HBc sequence shares at least 80 percent, and more preferably at least 90 percent, and even more preferably at least 95 percent, and most preferably 100 percent identity with a DNA sequence of SEQ ID NOs: 274, 275, 276, 277, 278 and 279, is defined as a DNA variant sequence. As is well-known, a nucleic acid sequence such as a contemplated nucleic acid sequence is expressed when operatively linked to an appropriate promoter in an appropriate expression system as discussed elsewhere herein.

An analog or analogous nucleic acid (DNA or RNA) sequence that encodes a contemplated chimer molecule is also contemplated as part of this invention. A chimer analog nucleic acid sequence or its complementary nucleic acid sequence encodes a HBc amino acid residue sequence that is at least 80 percent, and more preferably at least 90 percent, and most preferably is at least 95 percent identical to the HBc sequence portion from residue position 1 through residue position 136 shown in SEQ ID NOs: 246, 247, 248, 249, 250 and 251. This DNA or RNA is referred to herein as an “analog of” or “analogous to” a sequence of a nucleic acid of SEQ ID NOs: 274, 275, 276, 277, 278 and 279, and hybridizes with the nucleic acid sequence of SEQ ID NOs: 274, 275, 276, 277, 278 and 279 or their complements herein under moderate stringency hybridization conditions. A nucleic acid that encodes an analogous sequence, upon suitable transfection and expression, also produces a contemplated chimer.

Different hosts often have preferences for a particular codon to be used for encoding a particular amino acid residue. Such codon preferences are well known and a DNA sequence encoding a desired chimer sequence can be altered, using in vitro mutagenesis for example, so that host-preferred codons are utilized for a particular host in which the enzyme is to be expressed. In addition, one can also use the degeneracy of the genetic code to encode the HBc portion of a sequence of SEQ ID NOs: 246, 247, 248, 249, 250 or 251 that avoids substantial identity with a DNA of SEQ ID Nos: 274, 275, 276, 277, 278 or 279, or their complements. Thus, a useful analogous DNA sequence need not hybridize with the nucleotide sequences of SEQ ID NOs: 274, 275, 276, 277, 278 or 279 or a complement under conditions of moderate stringency, but can still provide a contemplated chimer molecule.

A recombinant nucleic acid molecule such as a DNA molecule, comprising a vector operatively linked to an exogenous nucleic acid segment (e.g., a DNA segment or sequence) that defines a gene that encodes a contemplated chimer, as discussed above, and a promoter suitable for driving the expression of the gene in a compatible host organism, is also contemplated in this invention. More particularly, also contemplated is a recombinant DNA molecule that comprises a vector comprising a promoter for driving the expression of the chimer in host organism cells operatively linked to a DNA segment that defines a gene for the HBc portion of a chimer or a DNA variant that has at least 90 percent identity to the chimer gene of SEQ ID NOs: 274, 275, 276, 277, 278 or 279 and hybridizes with that gene under moderate stringency conditions.

Further contemplated is a recombinant DNA molecule that comprises a vector containing a promoter for driving the expression of a chimer in host organism cells operatively linked to a DNA segment that is an analog nucleic acid sequence that encodes an amino acid residue sequence of a HBc chimer portion that is at least 80 percent identical, more preferably 90 percent identical, and most preferably 95 percent identical to the HBc portion of a sequence of SEQ ID NOs: 246, 247, 248, 249, 250 or 251. That recombinant DNA molecule, upon suitable transfection and expression in a host cell, provides a contemplated chimer molecule.

It is noted that because of the 30 amino acid residue N-terminal sequence of ground squirrel HBc does not align with any of the other HBc sequences, that sequence and its encoding nucleic acid sequences and their complements are not included in the above percentages of identity, nor are the portions of nucleic acid that encode that 30-residue sequence or its complement used in hybridization determinations. Similarly, sequences that are truncated at either or both of the HBc N- and C-termini are not included in identity calculations, nor are those sequences in which residues of the immunodominant loop are removed for insertion of a heterologous epitope. Thus, only those HBc-encoding bases or HBc sequence residues that are present in a chimer molecule are included and compared to an aligned nucleic acid or amino acid residue sequence in the identity percentage calculations.

Inasmuch as the coding sequences for the gene disclosed herein is illustrated in SEQ ID NOs: 274, 275, 276, 277, 278 and 279, isolated nucleic acid segments, preferably DNA sequences, variants and analogs thereof can be prepared by in vitro mutagenesis, as is well known in the art and discussed in Current Protocols In Molecular Biology, Ausabel et al. eds., John Wiley & Sons (New York: 1987) p. 8.1.1-8.1.6, that begin at the initial ATG codon for a gene and end at or just downstream of the stop codon for each gene. Thus, a desired restriction site can be engineered at or upstream of the initiation codon, and at or downstream of the stop codon so that other genes can be prepared, excised and isolated.

As is well known in the art, so long as the required nucleic acid, illustratively DNA sequence, is present, (including start and stop signals), additional base pairs can usually be present at either end of the segment and that segment can still be utilized to express the protein. This, of course, presumes the absence in the segment of an operatively linked DNA sequence that represses expression, expresses a further product that consumes the enzyme desired to be expressed, expresses a product that consumes a wanted reaction product produced by that desired enzyme, or otherwise interferes with expression of the gene of the DNA segment.

Thus, so long as the DNA segment is free of such interfering DNA sequences, a DNA segment of the invention can be about 500 to about 15,000 base pairs in length. The maximum size of a recombinant DNA molecule, particularly an expression vector, is governed mostly by convenience and the vector size that can be accommodated by a host cell, once all of the minimal DNA sequences required for replication and expression, when desired, are present. Minimal vector sizes are well known. Such long DNA segments are not preferred, but can be used.

DNA segments that encode the before-described chimer can be synthesized by chemical techniques, for example, the phosphotriester method of Matteucci et al. (1981) J. Am. Chem. Soc., 103:3185. Of course, by chemically synthesizing the coding sequence, any desired modifications can be made simply by substituting the appropriate bases for those encoding the native amino acid residue sequence. However, DNA segments including sequences discussed previously are preferred.

A contemplated HBc chimer can be produced (expressed) in a number of transformed host systems, typically host cells although expression in acellular, in vitro, systems is also contemplated. These host cellular systems include, but are not limited to, microorganisms such as bacteria transformed with recombinant bacteriophage, plasmid, or cosmid DNA expression vectors; yeast transformed with yeast expression vectors; insect cell systems infected with virus expression vectors (e.g. baculovirus); plant cell systems transformed with virus expression vectors (e.g. cauliflower mosaic virus; tobacco mosaic virus) or with bacterial expression vectors (e.g., Ti plasmid); or appropriately transformed animal cell systems such as CHO, VERO or COS cells. The invention is not limited by the host cell employed.

DNA segments containing a gene encoding the HBc chimer are preferably obtained from recombinant DNA molecules (plasmid vectors) containing that gene. Vectors capable of directing the expression of a chimer gene into the protein of a HBc chimer is referred to herein as an “expression vector”.

An expression vector contains expression control elements including the promoter. The chimer-coding gene is operatively linked to the expression vector to permit the promoter sequence to direct RNA polymerase binding and expression of the chimer-encoding gene. Useful in expressing the polypeptide coding gene are promoters that are inducible, viral, synthetic, constitutive as described by Poszkowski et al. (1989) EMBO J., 3:2719 and Odell et al. (1985) Nature, 313:810, as well as temporally regulated, spatially regulated, and spatiotemporally regulated as given in Chua et al. (1989) Science, 244:174-181.

One preferred promoter for use in prokaryotic cells such as E. coli is the Rec 7 promoter that is inducible by exogenously supplied nalidixic acid. A more preferred promoter is present in plasmid vector JHEX25 (available from Promega) that is inducible by exogenously supplied isopropyl-β-D-thiogalacto-pyranoside (IPTG). A still more preferred promoter, the tac promoter, is present in plasmid vector pKK223-3 and is also inducible by exogenously supplied IPTG. The pKK223-3 plasmid can be successfully expressed in a number of E. coli strains, such as XL-1, TB1, BL21 and BLR, using about 25 to about 100 μM IPTG for induction. Surprisingly, concentrations of about 25 to about 50 μM IPTG have been found to provide optimal results in 2 L shaker flasks and fermentors.

Several strains of Salmonella such as S. typhi and S. typhimurium and S. typhimurium-E. coli hybrids have been used to express immunogenic transgenes including prior HBc chimer particles both as sources of the particles for use as immunogens and as live, attenuated whole cell vaccines and inocula, and those expression and vaccination systems can be used herein. See, U.S. Pat. No. 6,024,961; U.S. Pat. No. 5,888,799; U.S. Pat. No. 5,387,744; U.S. Pat. No. 5,297,441; Ulrich et al., (1998) Adv. Virus Res., 50:141-182; Tacket et al., (August 1997) Infect. Immun., 65(8):3381-3385; Schodel et al., (February 1997) Behring Inst. Mitt., 98:114-119; Nardelli-Haefliger et al., (December 1996) Infect. Immun., 64(12):5219-5224; Londono et al., (April 1996) Vaccine, 14(6):545-552, and the citations therein.

Expression vectors compatible with eukaryotic cells, such as those compatible with yeast cells or those compatible with cells of higher plants or mammals, are also contemplated herein. Such expression vectors can also be used to form the recombinant DNA molecules of the present invention. Vectors for use in yeasts such as S. cerivisiae or Pichia pastoris can be episomal or integrating, as is well known. Eukaryotic cell expression vectors are well known in the art and are available from several commercial sources. Normally, such vectors contain one or more convenient restriction sites for insertion of the desired DNA segment and promoter sequences. Optionally, such vectors contain a selectable marker specific for use in eukaryotic cells. Exemplary promoters for use in S. cerevisiae include the S. cerevisiae phosphoglyceric acid kinase (PGK) promoter and the divergent promoters GAL 10 and GAL 1, whereas the alcohol oxidase gene (AOX1) is a useful promoter for Pichia pastoris.

For example, to produce chimers in the methylotrophic yeast, P. pastoris, a gene that encodes a desired chimer is placed under the control of regulatory sequences that direct expression of structural genes in Pichia. The resultant expression-competent forms of those genes are introduced into Pichia cells.

More specifically, the transformation and expression system described by Cregg et al. (1987) Biotechnology, 5:479-485; (1987) Molecular and Cellular Biology, 12:3376-3385 can be used. A gene for a chimer V12.Pf3.1 is placed downstream from the alcohol oxidase gene (AOX1) promoter and upstream from the transcription terminator sequence of the same AOX1 gene. The gene and its flanking regulatory regions are then introduced into a plasmid that carries both the P. pastoris HIS4 gene and a P. pastoris ARS sequence (Autonomously Replicating Sequence), which permit plasmid replication within P. pastoris cells [Cregg et al. (1987) Molecular and Cellular Biology, 12:3376-3385].

The vector also contains appropriate portions of a plasmid such as pBR322 to permit growth of the plasmid in E. coli cells. The resultant plasmid carrying a chimer gene, as well as the various additional elements described above, is illustratively transformed into a his4 mutant of P. pastoris; i.e. cells of a strain lacking a functional histidinol dehydrogenase gene.

After selecting transformant colonies on media lacking histidine, cells are grown on media lacking histidine, but containing methanol as described Cregg et al. (1987) Molecular and Cellular Biology, 12:3376-3385, to induce the AOX1 promoters. The induced AOX1 promoters cause expression of the chimer protein and the production of chimer particles in P. pastoris.

A contemplated chimer gene can also be introduced by integrative transformation, which does not require the use of an ARS sequence, as described by Cregg et al. (1987) Molecular and Cellular Biology, 12:3376-3385.

Production of chimer particles by recombinant DNA expression in mammalian cells is illustratively carried out using a recombinant DNA vector capable of expressing the chimer gene in Chinese hamster ovary (CHO) cells. This is accomplished using procedures that are well known in the art and are described in more detail in Sambrook et al., Molecular Cloning: A Laboratory Manual, 2^{nd} ed., Cold Spring Harbor Laboratories (1989).

In one illustrative example, the simian virus (SV40) based expression vector, pKSV-10 (Pharmacia Fine Chemicals, Piscataway, N.J.), is subjected to restriction endonuclease digestion by NcoI and HindIII. A NcoI/HindIII sequence fragment that encodes the desired HBc chimer prepared as described in Example 1 is ligated into the expression plasmid, which results in the formation of a circular recombinant expression plasmid denominated pSV-Pf.

The expression plasmid pSV-Pf contains an intact E. coli ampicillin resistance gene. E. coli RR101 (Bethesda Research Laboratories, Gaithersburg, Md.), when transformed with pSV-Pf, can thus be selected on the basis of ampicillin resistance for those bacteria containing the plasmid. Plasmid-containing bacteria are then cloned and the clones are subsequently screened for the proper orientation of the inserted coding gene into the expression vector.

The above obtained plasmid, pSV-Pf, containing the gene that encodes a desired HBc chimer is propagated by culturing E. coli containing the plasmid. The plasmid DNA is isolated from E. coli cultures as described in Sambrook et al., above.

Expression of a chimer is accomplished by the introduction of pSV-Pf into the mammalian cell line, e.g., CHO cells, using the calcium phosphate-mediated transfection method of Graham et al. (1973) Virol., 52:456, or a similar technique.

To help ensure maximal efficiency in the introduction of pSV-Pf into CHO cells in culture, the transfection is carried out in the presence of a second plasmid, pSV2NEO (ATCC #37149) and the cytotoxic drug G418 (GIBCO Laboratories, Grand Island, N.Y.) as described by Southern et al. (1982) J. Mol. Appl. Genet., 1:327. Those CHO cells that are resistant to G418 are cultured, have acquired both plasmids, pSV2NEO and pSV-Pf, and are designated CHO/pSV-Pf cells. By virtue of the genetic architecture of the pSV-Pf expression vector, a chimer is expressed in the resulting CHO/pSV-Pf cells and can be detected in and purified from the cytoplasm of these cells. The resulting composition containing cellular protein is separated on a column as discussed elsewhere herein.

The choice of which expression vector and ultimately to which promoter a chimer-encoding gene is operatively linked depends directly on the functional properties desired, e.g. the location and timing of protein expression, and the host cell to be transformed. These are well known limitations inherent in the art of constructing recombinant DNA molecules. However, a vector useful in practicing the present invention can direct the replication, and preferably also the expression (for an expression vector) of the chimer gene included in the DNA segment to which it is operatively linked.

In one preferred embodiment, the host that expresses the chimer is the prokaryote, E. coli, and a preferred vector includes a prokaryotic replicon; i.e., a DNA sequence having the ability to direct autonomous replication and maintenance of the recombinant DNA molecule extrachromosomally in a prokaryotic host cell transformed therewith. Such replicons are well known in the art.

Those vectors that include a prokaryotic replicon can also include a prokaryotic promoter region capable of directing the expression of a contemplated HBc chimer gene in a host cell, such as E. coli, transformed therewith. Promoter sequences compatible with bacterial hosts are typically provided in plasmid vectors containing one or more convenient restriction sites for insertion of a contemplated DNA segment. Typical of such vector plasmids are pUC8, pUC9, and pBR329 available from Biorad Laboratories, (Richmond, Calif.) and pPL and pKK223-3 available from Pharmacia, Piscataway, N.J.

Typical vectors useful for expression of genes in cells from higher plants and mammals are well known in the art and include plant vectors derived from the tumor-inducing (Ti) plasmid of Agrobacterium tumefaciens described by Rogers et al. (1987) Meth. in Enzymol., 153:253-277 and mammalian expression vectors pKSV-10, above, and pCI-neo (Promega Corp., #E1841, Madison, Wis.). However, several other expression vector systems are known to function in plants including pCaMVCN transfer control vector described by Fromm et al. (1985) Proc. Natl. Acad. Sci. USA, 82:58-24. Plasmid pCaMVCN (available from Pharmacia, Piscataway, N.J.) includes the cauliflower mosaic virus CaMV 35S promoter.

The above plant expression systems typically provide systemic or constitutive expression of an inserted transgene. Systemic expression can be useful where most or all of a plant is used as the source to a contemplated chimer molecule or resultant particles or where a large part of the plant is used to provide an oral vaccine. However, it can be more efficacious to express a chimer molecule or particles in a plant storage organ such as a root, seed or fruit from which the particles can be more readily isolated or ingested.

One manner of achieving storage organ expression is to use a promoter that expresses its controlled gene in one or more preselected or predetermined non-photosynthetic plant organs. Expression in one or more preselected storage organs with little or no expression in other organs such as roots, seed or fruit versus leaves or stems is referred to herein as enhanced or preferential expression. An exemplary promoter that directs expression in one or more preselected organs as compared to another organ at a ratio of at least 5:1 is defined herein as an organ-enhanced promoter. Expression in substantially only one storage organ and substantially no expression in other storage organs is referred to as organ-specific expression; i.e., a ratio of expression products in a storage organ relative to another of about 100:1 or greater indicates organ specificity. Storage organ-specific promoters are thus members of the class of storage organ-enhanced promoters.

Exemplary plant storage organs include the roots of carrots, taro or manioc, potato tubers, and the meat of fruit such as red guava, passion fruit, mango, papaya, tomato, avocado, cherry, tangerine, mandarin, palm, melons such cantaloupe and watermelons and other fleshy fruits such as squash, cucumbers, mangos, apricots, peaches, as well as the seeds of maize (corn), soybeans, rice, oil seed rape and the like.

The CaMV 35S promoter is normally deemed to be a constitutive promoter. However, recent research has shown that a 21-bp region of the CaMV 35S promoter, when operatively linked into another, heterologous usual green tissue promoter, the rbcS-3A promoter, can cause the resulting chimeric promoter to become a root-enhanced promoter. That 21-bp sequence is disclosed in U.S. Pat. No. 5,023,179. The chimeric rbcS-3A promoter containing the 21-bp insert of U.S. Pat. No. 5,023,179 is a useful root-enhanced promoter herein.

A similar root-enhanced promoter, that includes the above 21-bp segment is the −90 to +8 region of the CAMV 35S promoter itself. U.S. Pat. No. 5,110,732 discloses that that truncated CaMV 35S promoter provides enhanced expression in roots and the radical of seed, a tissue destined to become a root. That promoter is also useful herein.

Another useful root-enhanced promoter is the −1616 to −1 promoter of the oil seed rape (Brassica napus L.) gene disclosed in PCT/GB92/00416 (WO 91/13922 published Sep. 19, 1991). E. coli DH5.alpha. harboring plasmid pRlambdaS4 and bacteriophage lambda.beta.l that contain this promoter were deposited at the National Collection of Industrial and Marine Bacteria, Aberdeen, GB on Mar. 8, 1990 and have accession numbers NCIMB40265 and NCIMB40266. A useful portion of this promoter can be obtained as a 1.0 kb fragment by cleavage of the plasmid with HaeIII.

A preferred root-enhanced promoter is the mannopine synthase (mas) promoter present in plasmid pKan2 described by DiRita and Gelvin (1987) Mol. Gen. Genet, 207:233-241. This promoter is removable from its plasmid pKan2 as a XbaI-XbaII fragment.

The preferred mannopine synthase root-enhanced promoter is comprised of the core mannopine synthase (mas) promoter region up to position −138 and the mannopine synthase activator from −318 to −213, and is collectively referred to as AmasPmas. This promoter has been found to increase production in tobacco roots about 10- to about 100-fold compared to leaf expression levels.

Another root specific promoter is the about 500 bp 5′ flanking sequence accompanying the hydroxyproline-rich glycopeprotein gene, HRGPnt3, expressed during lateral root initiation and reported by Keller et al. (1989) Genes Dev., 3:1639-1646. Another preferred root-specific promoter is present in the about −636 to −15′ flanking region of the tobacco root-specific gene TORBF reported by Yamamoto et al. (1991) Plant Cell, 3:371-381. The cis-acting elements regulating expression are more specifically located by those authors in the region from about −636 to about −299 5′ from the transcription initiation site. Yamamoto et al. reported steady state mRNA production from the TORBF gene in roots, but not in leaves, shoot meristems or stems.

Still another useful storage organ-specific promoter are the 5′ and 3′ flanking regions of the fruit-ripening gene E8 of the tomato, Lycopersicon esculentum. These regions and their cDNA sequences are illustrated and discussed in Deikman et al. (1988) EMBO J., 7(11):3315-3320 and (1992) Plant Physiol., 100:2013-2017.

Three regions are located in the 2181 bp of the 5′ flanking sequence of the gene and a 522 bp sequence 3′ to the poly (A) addition site appeared to control expression of the E8 gene. One region from −2181 to −1088 is required for activation of E8 gene transcription in unripe fruit by ethylene and also contributes to transcription during ripening. Two further regions, −1088 to −863 and −409 to −263, are unable to confer ethylene responsiveness in unripe fruit but are sufficient for E8 gene expression during ripening.

The maize sucrose synthase-1 (Sh) promoter that in corn expresses its controlled enzyme at high levels in endosperm, at much reduced levels in roots and not in green tissues or pollen has been reported to express a chimeric reporter gene, β-glucuronidase (GUS), specifically in tobacco phloem cells that are abundant in stems and roots. Yang et al. (1990) Proc. Natl. Acad. Sci., U.S.A., 87:4144-4148. This promoter is thus useful for plant organs such as fleshy fruits like melons, e.g. cantaloupe, or seeds that contain endosperm and for roots that have high levels of phloem cells.

Another exemplary tissue-specific promoter is the lectin promoter, which is specific for seed tissue. The lectin protein in soybean seeds is encoded by a single gene (Le1) that is only expressed during seed maturation and accounts for about 2 to about 5 percent of total seed mRNA. The lectin gene and seed-specific promoter have been fully characterized and used to direct seed specific expression in transgenic tobacco plants. See, e.g., Vodkin et al. (1983) Cell, 34:1023 and Lindstrom et al. (1990) Developmental Genetics, 11:160.

A particularly preferred tuber-specific expression promoter is the 5′ flanking region of the potato patatin gene. Use of this promoter is described in Twell et al. (1987) Plant Mol. Biol., 9:365-375. This promoter is present in an about 406 bp fragment of bacteriophage LPOTI. The LPOTI promoter has regions of over 90 percent homology with four other patatin promoters and about 95 percent homology over all 400 bases with patatin promoter PGT5. Each of these promoters is useful herein. See, also, Wenzler et al. (1989) Plant Mol. Biol., 12:41-50.

Still further organ-enhanced and organ-specific promoter are disclosed in Benfey et al. (1988) Science, 244:174-181.

Each of the promoter sequences utilized is substantially unaffected by the amount of chimer molecule or particles in the cell. As used herein, the term “substantially unaffected” means that the promoter is not responsive to direct feedback control (inhibition) by the chimer molecules or particles accumulated in transformed cells or transgenic plant.

Transfection of plant cells using Agrobacterium tumefaciens is typically best carried out on dicotyledonous plants. Monocots are usually most readily transformed by so-called direct gene transfer of protoplasts. Direct gene transfer is usually carried out by electroportation, by polyethyleneglycol-mediated transfer or bombardment of cells by microprojectiles carrying the needed DNA. These methods of transfection are well-known in the art and need not be further discussed herein. Methods of regenerating whole plants from transfected cells and protoplasts are also well-known, as are techniques for obtaining a desired protein from plant tissues. See, also, U.S. Pat. Nos. 5,618,988 and 5,679,880 and the citations therein.

A transgenic plant formed using Agrobacterium transformation, electroportation or other methods typically contains a single gene on one chromosome. Such transgenic plants can be referred to as being heterozygous for the added gene. However, inasmuch as use of the word “heterozygous” usually implies the presence of a complementary gene at the same locus of the second chromosome of a pair of chromosomes, and there is no such gene in a plant containing one added gene as here, it is believed that a more accurate name for such a plant is an independent segregant, because the added, exogenous chimer molecule-encoding gene segregates independently during mitosis and meiosis. A transgenic plant containing an organ-enhanced promoter driving a single structural gene that encodes a contemplated HBc chimeric molecule; i.e., an independent segregant, is a preferred transgenic plant.

More preferred is a transgenic plant that is homozygous for the added structural gene; i.e., a transgenic plant that contains two added genes, one gene at the same locus on each chromosome of a chromosome pair. A homozygous transgenic plant can be obtained by sexually mating (selfing) an independent segregant transgenic plant that contains a single added gene, germinating some of the seed produced and analyzing the resulting plants produced for enhanced chimer particle accumulation relative to a control (native, non-transgenic) or an independent segregant transgenic plant. A homozygous transgenic plant exhibits enhanced chimer particle accumulation as compared to both a native, non-transgenic plant and an independent segregant transgenic plant.

It is to be understood that two different transgenic plants can also be mated to produce offspring that contain two independently segregating added, exogenous (heterologous) genes. Selfing of appropriate progeny can produce plants that are homozygous for both added, exogenous genes that encode a chimeric HBc molecule. Back-crossing to a parental plant and out-crossing with a non-transgenic plant are also contemplated.

A transgenic plant of this invention thus has a heterologous structural gene that encodes a contemplated chimeric HBc molecule. A preferred transgenic plant is an independent segregant for the added heterologous chimeric HBc structural gene and can transmit that gene to its progeny. A more preferred transgenic plant is homozygous for the heterologous gene, and transmits that gene to all of its offspring on sexual mating.

Inasmuch as a gene that encodes a chimeric HBc molecule does not occur naturally in plants, a contemplated transgenic plant accumulates chimeric HBc molecule particles in a greater amount than does a non-transformed plant of the same type or strain when both plants are grown under the same conditions.

The phrase “same type” or “same strain” is used herein to mean a plant of the same cross as or a clone of the untransformed plant. Where alleic variations among siblings of a cross are small, as with extensively inbred plant, comparisons between siblings can be used or an average arrived at using several siblings. Otherwise, clones are preferred for the comparison.

Seed from a transgenic plant is grown in the field greenhouse, window sill or the like, and resulting sexually mature transgenic plants are self-pollinated to generate true breeding plants. The progeny from these plants become true breeding lines that are evaluated for chimeric HBc molecule particle accumulation, preferably in the field, under a range of environmental conditions.

A transgenic plant homozygous for chimeric HBc molecule particle accumulation is crossed with a parent plant having other desired traits. The progeny, which are heterozygous or independently segregatable for chimeric HBc molecule particle accumulation, are backcrossed with one or the other parent to obtain transgenic plants that exhibit chimeric HBc molecule particle accumulation and the other desired traits. The backcrossing of progeny with the parent may have to be repeated more than once to obtain a transgenic plant that possesses a number of desirable traits.

An insect cell system can also be used to express a HBc chimer. For example, in one such system Autographa californica nuclear polyhedrosis virus (AcNPV) or baculovirus is used as a vector to express foreign genes in Spodoptera frugiperda cells or in Trichoplusia larvae.

The sequences encoding a chimer can be cloned into a non-essential region of the virus, such as the polyhedrin gene, and placed under control of the polyhedrin promoter. Successful insertion of chimer sequence renders the polyhedrin gene inactive and produces recombinant virus lacking coat protein. The recombinant viruses can then be used to infect, for example, S. Frugiperda cells or Trichoplusia larvae in which the HBc chimer can be expressed. E. Engelhard et al. (1994) Proc. Natl. Acad. Sci., USA, 91:3224-3227; and V. Luckow, Insect Cell Expression Technology, pp. 183-218, in Protein Engineering: Principles and Practice, J. L. Cleland et al. eds., Wiley-Liss, Inc, 1996). Heterologous genes placed under the control of the polyhedrin promoter of the Autographa californica nuclear polyhedrosis virus (AcNPV) are often expressed at high levels during the late stages of infection.

Recombinant baculoviruses containing the chimeric gene are constructed using the baculovirus shuttle vector system (Luckow et al. (1993) J. Virol., 67:4566-4579], sold commercially as the Bac-To-Bac™ baculovirus expression system (Life Technologies). Stocks of recombinant viruses are prepared and expression of the recombinant protein is monitored by standard protocols (O'Reilly et al., Baculovirus Expression Vectors: A Laboratory Manual, W.H. Freeman and Company, New York, 1992; and King et al., The Baculovirus Expression System: A Laboratory Guide, Chapman & Hall, London, 1992).

A variety of methods have been developed to operatively link DNA to vectors via complementary cohesive termini or blunt ends. For instance, complementary homopolymer tracts can be added to the DNA segment to be inserted into the vector DNA. The vector and DNA segment are then joined by hydrogen bonding between the complementary homopolymeric tails to form recombinant DNA molecules.

Alternatively, synthetic linkers containing one or more restriction endonuclease sites can be used to join the DNA segment to the expression vector, as noted before. The synthetic linkers are attached to blunt-ended DNA segments by incubating the blunt-ended DNA segments with a large excess of synthetic linker molecules in the presence of an enzyme that is able to catalyze the ligation of blunt-ended DNA molecules, such as bacteriophage T4 DNA ligase.

Thus, the products of the reaction are DNA segments carrying synthetic linker sequences at their ends. These DNA segments are then cleaved with the appropriate restriction endonuclease and ligated into an expression vector that has been cleaved with an enzyme that produces termini compatible with those of the synthetic linker. Synthetic linkers containing a variety of restriction endonuclease sites are commercially available from a number of sources including New England BioLabs, Beverly, Mass. A desired DNA segment can also be obtained using PCR technology in which the forward and reverse primers contain desired restriction sites that can be cut after amplification so that the gene can be inserted into the vector. Alternatively PCR products can be directly cloned into vectors containing T-overhangs (Promega Corp., A3600, Madison, Wis.) as is well known in the art.

The expressed chimeric protein self-assembles into particles within the host cells, whether in single cells or in cells within a multicelled host. The particle-containing cells are harvested using standard procedures, and the cells are lysed using a French pressure cell, lysozyme, sonicator, bead beater or a microfluidizer (Microfluidics International Corp., Newton Mass.). After clarification of the lysate, particles are precipitated with 45% ammonium sulfate, resuspended in 20 mM sodium phosphate, pH 6.8 and dialyzed against the same buffer. The dialyzed material is clarified by brief centrifugation and the supernatant subjected to gel filtration chromatography using Sepharose® CL-4B. Particle-containing fractions are identified, subjected to hydroxyapatite chromatography, and reprecipitated with ammonium sulfate prior to resuspension, dialysis and sterile filtration and storage at −70° C.

HBc Chimer Conjugates

Any hapten to which a B cell or T cell response is desired can be linked to a contemplated HBc chimer or chimer particle such as a chimer particle containing a heterologous linker residue such as a lysine, glutamic or aspartic acid, cysteine or tyrosine in the loop region of Domain II and an added cysteine residue in Domain IV to form a HBc chimer conjugate. The hapten of interest typically is a B cell immunogen. The hapten can be a polypeptide, a carbohydrate (saccharide; i.e., oligo- or polysaccharide), or a non-polypeptide, non-carbohydrate chemical such as 2,4-dinitrobenzene or a medicament such as cocaine or nicotine. A HBc chimer particle conjugate so formed is useful as an inoculum or vaccine, as is discussed hereinafter. Because the chimer protein self assembles upon expression and a conjugate is formed after expression, conjugate formation is typically done using the assembled particles as compared to the free protein molecules.

Methods for operatively linking individual haptens to a protein or polypeptide through an amino acid residue side chain of the protein or polypeptide to form a pendently-linked immunogenic conjugate, e.g., a branched-chain polypeptide polymer, are well known in the art. Those methods include linking through one or more types of functional groups on various side chains and result in the carrier protein polypeptide backbone (here, a HBc chimer) within the particle being pendently linked—covalently linked (coupled)—to the hapten but separated by at least one side chain.

Methods for linking carrier proteins to haptens using each of the above functional groups are described in Erlanger, (1980) Method of Enzymology, 70:85; Aurameas et al., (1978) Scand. J. Immunol., Vol. 8, Suppl. 7, 7-23 and U.S. Pat. No. 4,493,795 to Nestor et al. In addition, a site-directed coupling reaction, as described in Rodwell et al. (1985) Biotech., 3:889-894 can be carried out so that the biological activity of the polypeptides is not substantially diminished.

Furthermore, as is well known in the art, both the HBc protein and a polypeptide hapten can be used in their native form or their functional group content can be modified by succinylation of lysine residues or reaction with cysteine-thiolactone. A sulfhydryl group can also be incorporated into either carrier protein or conjugate by reaction of amino functional groups with 2-iminothiolane, the N-hydroxysuccinimide ester of 3-(3-dithiopyridyl)-propionate, or other reagents known in the art.

The HBc chimer or hapten can also be modified to incorporate a spacer arm, such as hexamethylene diamine or another bifunctional molecule, to facilitate the pendent linking. Such a procedure is discussed below.

Methods for covalent bonding of a polypeptide hapten are extremely varied and are well known by workers skilled in the immunological arts. For example, following U.S. Pat. No. 4,818,527, m-maleimidobenzoyl-N-hydroxysuccinimide ester (ICN Biochemicals, Inc., Costa Mesa, Calif.) or succinimidyl 4-(N-maleimidomethyl)cyclohexane-1-carboxylate (SMCC, Pierce Chemical Co., Rockford, Ill.) is reacted with an appropriate HBc chimer to form an activated carrier.

That activated carrier is then reacted with a hapten such as a sulfhydryl-terminated hapten or a polypeptide that either contains a terminal cysteine or to which an additional amino- or carboxy-terminal cysteine residue has been added to form a covalently bonded HBc chimer conjugate. As an alternative example, the amino group of a polypeptide hapten can be first reacted with N-succinimidyl 3-(2-pyridylthio)propionate (SPDP, Pharmacia, Piscataway, N.J.), and that thiol-containing polypeptide can be reacted with the activated carrier after reduction. Of course, the sulfur-containing moiety and double bond-containing Michael acceptor can be reversed. These reactions are described in the supplier's literature, and also in Kitagawa, et al. (1976) J. Biochem., 79:233 and in Lachmann et al., in 1986 Synthetic Peptides as Antigens, (Ciba Foundation Symposium 119), pp. 25-40 (Wiley, Chichester: 1986).

U.S. Pat. No. 4,767,842 teaches several modes of covalent attachment between a carrier and polypeptide that are useful here. In one method, tolylene diisocyanate is reacted with the carrier in a dioxane-buffer solvent at zero degrees C. to form an activated carrier. A polypeptide hapten is thereafter admixed and reacted with the activated carrier to form the covalently bonded HBc chimer conjugate.

Particularly useful are a large number of heterobifunctional agents that form a disulfide link at one functional group end and an amide link at the other, including N-succidimidyl-3-(2-pyridyldithio)-propionate (SPDP), discussed before that creates a disulfide linkage between itself and a thiol in either the HBc chimer or the hapten. Exemplary reagents include a cysteine residue in a polypeptide hapten and an amine on the coupling partner such as the ε-amine of a lysine or other free amino group in the carrier protein. A variety of such disulfide/amide forming agents are known. See for example Immun. Rev. (1982) 62:185.

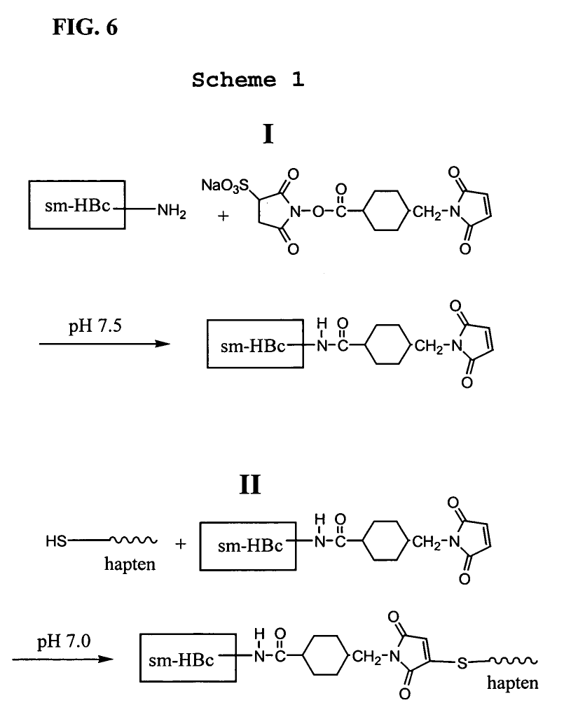

Other bifunctional coupling agents form a thioether rather than a disulfide linkage. Many of these thioether-forming agents are commercially available and include reactive esters of 6-maleimidocaproic acid, 2-bromoacetic acid, 2-iodoacetic acid, 4-(N-maleimidomethyl)cyclohexane-1-carboxylic acid and the like. The carboxyl groups can be activated by combining them with succinimide or 1-hydroxy-2-nitro-4-sulfonic acid, sodium salt. The particularly preferred coupling agent for the method of this invention is succinimidyl 4-(N-maleimidomethyl)cyclohexane-1-carboxylate (SMCC) obtained from Pierce Chemical Co., Rockford, Ill. The foregoing list is not meant to be exhaustive, and modifications of the named compounds can clearly be used. FIG. 6 provides a schematic representation (Scheme 1) of the formation of a HBc activated carrier using SMCC (I) and the subsequent reaction of that activated carrier with a sulfhydryl-terminated hapten (II).

A polypeptide hapten can be obtained in a number of ways well known in the art. Usual peptide synthesis techniques can be readily utilized. For example, recombinant and PCR-based techniques to produce longer peptides are useful. Because the desired sequences are usually relatively short, solid phase chemical synthesis is useful.

Exemplary polypeptide haptens are shown in Tables A and B hereinbefore. Each of those polypeptides can be utilized via its N-terminal amino group, or by use of an additional N-terminal cysteine that is not shown in the table.

Related chemistry is used to couple what may be called “chemical compounds” to carrier proteins. Typically, an appropriate functional group for coupling is designed into the chemical compound. An exemplary chemical hapten to which induced antibodies protect against Streptococcus pneumoniae is 6-O-phosphocholine hydroxyhexanoate. Fischer et al. (1995) J. Immunol., 154:3373-3382. The table below provides further exemplary chemical haptens.

Chemical Haptens

| Chemical Hapten | Citation |
|---|---|
| piperidine N-oxide | U.S. Pat. No. 5,304,252 |
| phospholactone or | U.S. Pat. No. 5,248,611 |
| lactamide | |
| metal ion complexes | U.S. Pat. No. 5,236,825 |
| [2.2.1] or [7.2.2] | U.S. Pat. No. 5,208,152 |
| bicyclic ring | |
| compounds | |
| ionically charged | U.S. Pat. No. 5,187,086 |
| hydroxyl-containing | |
| compounds | |
| phosphonate analogs | U.S. Pat. No. 5,126,258 |
| of carboxylate | |
| esters | |
| cocaine analogs | Carrera et al., (1995) |
| | Nature 378:725 |

There are many methods known in the art to couple carrier proteins to polysaccharides. Aldehyde groups can be prepared on either the reducing end [Anderson (1983) Infect. Immun., 39:233-238; Jennings, et al. (1981) J. Immunol., 127:1011-1018; Poren et al. (1985) Mol. Immunol., 22:907-919] or the terminal end [Anderson et al. (1986) J. Immunol., 137:1181-1186; Beuvery et al. (1986) Dev. Bio. Scand., 65:197-204] of an oligosaccharide or relatively small polysaccharide, which can be linked to the carrier protein via reductive amination.

Large polysaccharides can be conjugated by either terminal activation [Anderson et al. (1986) J. Immunol., 137:1181-1186] or by random activation of several functional groups along the polysaccharide chain [Chu et al. (1983) Infect. Immun., 40:245-256; Gordon, U.S. Pat. No. 4,619,828 (1986); Marburg, U.S. Pat. No. 4,882,317 (1989)]. Random activation of several functional groups along the polysaccharide chain can lead to a conjugate that is highly cross-linked due to random linkages along the polysaccharide chain. The optimal ratio of polysaccharide to carrier protein depend on the particular polysaccharide, the carrier protein, and the conjugate used.

Detailed reviews of methods of conjugation of saccharide to carrier proteins can be found in Dick et al., in Contributions to Microbiology and Immunology, Vol. 10, Cruse et al., eds., (S. Karger: 1989), pp. 48-114; Jennings et al., in Neoglycoconjugates: Preparation and Applications, Lee et al., eds., (Academic Press: 1994), pp. 325-371; Aplin et al., (1981) CRC Crit. Rev. Biochem., 10:259-306; and Stowell et al. (1980) Adv. Carbohydr. Chem. Biochem., 37:225-281.

The carbohydrate itself can be synthesized by methods known in the art, for example by enzymatic glycoprotein synthesis as described by Witte et al. (1997) J. Am. Chem. Soc., 119:2114-2118.

Several oligosaccharides, synthetic and semi-synthetic, and natural, are discussed in the following paragraphs as examples of oligosaccharides that are contemplated haptens to be used in making a HBc conjugate of the present invention.

An oligosaccharide hapten suitable for preparing vaccines for the treatment of Haemophilus influenza type b (Hib) is made up of from 2 to 20 repeats of D-ribose-D-ribitol-phosphate (I, below), D-ribitol-phosphate-D-ribose (II, below), or phosphate-D-ribose-D-ribitol (III, below). Eduard C. Beuvery et al., EP-0 276 516-B1.

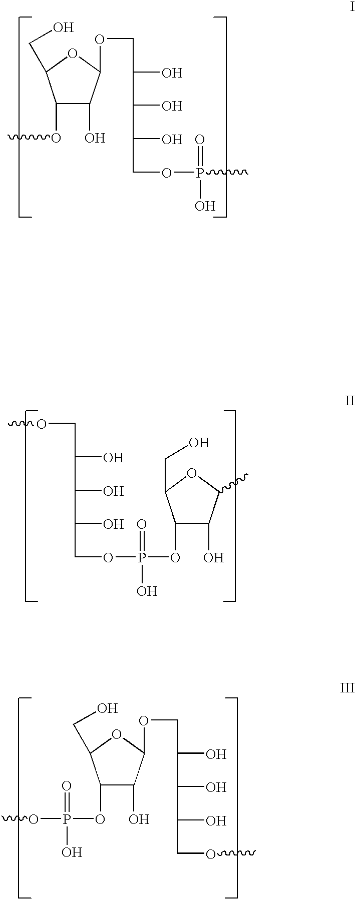

U.S. Pat. No. 4,220,717 also discloses a polyribosyl ribitol phosphate (PRP) hapten for Haemophilus influenzae type b.

Peterson et al. (1998) Infect. Immun., 66(8):3848-3855, disclose a trisaccharide hapten, αKdo(2 8)αKdo(2 4)αKdo, that provides protection from Chlamydia pneumoniae. Chlamydia pneumoniae is a cause of human respiratory infections ranging from pharyngitis to fatal pneumonia. Kdo is 3-deoxy-D-manno-oct-2-ulosonic acid.

Andersson et al., EP-0 126 043-A1, disclose saccharides that can be used in the treatment, prophylaxis or diagnosis of bacterial infections caused by Streptococci pneumoniae. One class of useful saccharides is derived from the disaccharide GlcNAcβ1 3Gal. Andersson et al. also reported neolactotetraosylceramide to be useful, which is Galβ1 4GlcNAcpβ1 3Galβ1 4Glc-Cer.

McKenney et al. (1999) Science, 284:1523-1527, disclose a polysaccharide, poly-N-succinyl β1 6GlcN (PNSG) that provides protection from Staphylococcus aureus. S. aureus is a common cause of community-acquired infections, including endocarditis, osetemylitis, septic arthritis, pneumonia, and abscesses.

European Patent No. 0 157 899-B1, the disclosures of which are incorporated herein by reference, discloses the isolation of pneumococcal polysaccharides that are useful in the present invention. The following table lists the pneumococcal culture types that produce capsular polysaccharides useful as haptens in the present invention.

Polysaccharide Hapten Sources

| Danish Type | U.S. | 1978 ATCC Catalogue |
|---|---|---|
| Nomenclature | Nomenclature | Number |
| 1 | 1 | 6301 |
| 2 | 2 | 6302 |
| 3 | 3 | 6303 |
| 4 | 4 | 6304 |
| 5 | 5 | |
| 6A | 6 | 6306 |
| 6B | 26 | 6326 |
| 7F | 51 | 10351 |
| 8 | 8 | 6308 |
| 9N | 9 | 6309 |
| 9V | 68 | |
| 10A | 34 | |
| 11A | 43 | |
| 12F | 12 | 6312 |
| 14 | 14 | 6314 |
| 15B | 54 | |
| 17F | 17 | |
| 18C | 56 | 10356 |
| 19A | 57 | |
| 19F | 19 | 6319 |
| 20 | 20 | 6320 |
| 22F | 22 | |
| 23F | 23 | 6323 |
| 25 | 25 | 6325 |
| 33F | 70 | |

Moraxella (Branhamella) catarrhalis is a reported cause of otitis media and sinusitis in children and lower respiratory tract infections in adults. The lipid A portion of the lipooligo-saccharide surface antigen (LOS) of the bacterium is cleaved at the 3-deoxy-D-manno-octulosonic acid-glucosamine linkage. The cleavage product is treated with mild-alkali to remove ester-linked fatty acids, while preserving amide-linked fatty acids to yield detoxified lipopolysaccharide (dLOS) from M. catarrhalis. The dLOS is not immunogenic until it is attached to a protein carrier. Xin-Xing Gu et al. (1998) Infect. Immun., 66(5):1891-1897.

Group B streptococci (GBS) is a cause of sepsis, meningitis, and related neurologic disorders in humans. The Capsular polysaccharide-specific antibodies are known to protect human infants from infection. Jennings et al., U.S. Pat. No. 5,795,580. The repeating unit of the GBS capsular polysaccharide type II is: 4)-β-D-GlcpNAc-(1 3)-[β-D-Galp(1 6)]-β-D-Galp(1 4)-β-D-Glcp-(1 3)-β-D-Glcp-(1 2)-[α-D-NeupNAc(2 3)]-β-D-Galp-(1, where the bracketed portion is a branch connected to the immediately following unbracketed subunit. The repeating unit of GBS capsular polysaccharide type V is: 4)-[α-D-NeupNAc-(2 3)-β-D-Galp-(1 4)-β-D-GlcpNAc-(1 6)]-α-D-Glcp-(1 4)-[β-D-Glcp-(1 3)]-β-D-Galp-(1 4)-β-D-Glcp-(1.

European patent application No. EU-0 641 568-A1, Brade, discloses the method of obtaining ladder-like banding pattern antigen from Chlamydia trachomatis, pneumoniae and psittaci.

Slovin et al., (1999) Proc. Natl. Acad. Sci., U.S.A., 96(10):5710-5715 report use of a synthetic oligosaccharide, globo H, linked to KLH as a carrier in the preparation of a vaccine used against prostate cancer. Similarly, Helling et al., (July 1995) Cancer Res., 55:2783-2788 report the use of KLH-linked G_{M2} in a vaccine for treating patients with melanoma. The latter vaccine was prepared by ozone cleavage of the ceramide double bond of G_{M2}, introduction of an aldehyde group and reductive alkylation onto KLH. A similar procedure can be utilized with a contemplated chimer particle.

Oligosaccharidal portions of sphingolipids such as globosides and gangliosides that are present on the surface of other tumor cells as well as normal cells such as melanoma, neuroblastoma and healthy brain cells can similarly be used herein as a hapten. The oligosaccharide portion of the globoside globo H has the structure Fucα-(1 2)-Galβ(1 3)-GalNAcβ-(1 3)-Galα-(1 4)-Galβ-(1 4)Glc, whereas the saccharide protions of gangliosides G_{M2}, G_{M1} and G_{D1a} have the following structures: GalNAcβ-(1 4)-[NeuAcα-(2 3)]-Galβ-(14)-Glc; Galβ-(1 3)-GalNAcβ-(1 4)-[NeuAcα-(2 3)]-Galβ-(1 4)-Glc; and NeuAc-(2 3)-Galβ-(1 3)-GalNAcβ-(1 4)-[NeuAcα-(2 3)]-Galβ-(1 4)-Glc, respectively.

U.S. Pat. No. 4,356,170 discloses the preparation of useful polysaccharides that are reduced and then oxidized to form compounds having terminal aldehyde groups that can be reductively aminated onto free amine groups of carrier proteins such as tetanus toxoid and diphtheria toxoid with or without significant cross-linking. Exemplary useful bacterial polysaccharides include β-hemolytic streptococci, Haemophilus influenza, meningococci, pneumococci and E. coli. Rather than reductively aminating the particles, a linker arm such as that provided by an ε-amino C₂-C₈ alkylcarboxylic acid can be reductively aminated on to the polysaccharide, followed by linkage to the particles using a water-soluble carbodiimide.

Inocula and Vaccines

In yet another embodiment of the invention, a HBc chimer particle or HBc chimer particle conjugate with a hapten is used as the immunogen of an inoculum that induces a B cell or T cell response (stimulation) in an inoculated host animal such as production of antibodies that immunoreact with the heterologous epitope or hapten or T cell activation, or as a vaccine to provide protection against the pathogen from which the heterologous epitope or the hapten is derived.

T cell activation can be measured by a variety of techniques. In usual practice, a host animal is inoculated with a contemplated HBc chimer particle vaccine or inoculum, and peripheral mononuclear blood cells (PMBC) are thereafter collected. Those PMBC are then cultured in vitro in the presence of the T cell immunogen for a period of about three to five days. The cultured PMBC are then assayed for proliferation or secretion of a cytokine such as IL-2, GM-CSF of IFN-γ. Assays for T cell activation are well known in the art. See, for example, U.S. Pat. No. 5,478,726 and the art cited therein.

Using antibody formation as exemplary, a contemplated inoculum or vaccine comprises an immunogenic effective amount of HBc chimer particles or HBc chimer particle conjugates that are dissolved or dispersed in a pharmaceutically acceptable diluent composition that typically also contains water. When administered to a host animal in need of immunization or in which antibodies are desired to be induced such as a mammal (e.g., a mouse, dog, goat, sheep, horse, bovine, monkey, ape, or human) or bird (e.g., a chicken, turkey, duck or goose), an inoculum induces antibodies that immunoreact with the conjugated (pendently-linked) hapten. Those antibodies also preferably bind to the protein or saccharide of the B cell immunogen.

A vaccine is a type of inoculum in which the heterologous B cell epitope or conjugated hapten corresponds to a portion of a protein or saccharidal structure that is related to a disease state, as is an exemplary malarial B cell sequence related to a malarial pathogen. The vaccine-induced antibodies not only immunoreact with the epitope or hapten or activated T cells respond to that heterologous epitope or hapten, but also immunoreact with the pathogen or diseased cell in vivo, and provide protection from that disease state.

The amount of recombinant HBc chimer immunogen utilized in each immunization is referred to as an immunogenic effective amount and can vary widely, depending inter alia, upon the recombinant HBc chimer immunogen, mammal immunized, and the presence of an adjuvant in the vaccine, as discussed below. Immunogenic effective amounts for a vaccine and an inoculum provide the protection or antibody activity, respectively, discussed hereinbefore.

Vaccines or inocula typically contain a recombinant HBc chimer immunogen concentration of about 1 microgram to about 1 milligram per inoculation (unit dose), and preferably about 10 micrograms to about 50 micrograms per unit dose. The term “unit dose” as it pertains to a vaccine or inoculum of the present invention refers to physically discrete units suitable as unitary dosages for animals, each unit containing a predetermined quantity of active material calculated to individually or collectively produce the desired immunogenic effect in association with the required diluent; i.e., carrier, or vehicle.

Vaccines or inocula are typically prepared from a recovered recombinant HBc chimer immunogen by dispersing the immunogen, preferably in particulate form, in a physiologically tolerable (acceptable) diluent vehicle such as water, saline phosphate-buffered saline (PBS), acetate-buffered saline (ABS), Ringer's solution or the like to form an aqueous composition. The diluent vehicle can also include oleaginous materials such as peanut oil, squalane or squalene as is discussed hereinafter.

The preparation of inocula and vaccines that contain proteinaceous materials as active ingredients is also well understood in the art. Typically, such inocula or vaccines are prepared as parenterals, either as liquid solutions or suspensions; solid forms suitable for solution in, or suspension in, liquid prior to injection can also be prepared. The preparation can also be emulsified, which is particularly preferred.

The immunogenic active ingredient is often mixed with excipients that are pharmaceutically acceptable and compatible with the active ingredient. Suitable excipients are, for example, water, saline, dextrose, glycerol, ethanol, or the like and combinations thereof. In addition, if desired, an inoculum or vaccine can contain minor amounts of auxiliary substances such as wetting or emulsifying agents, pH buffering agents that enhance the immunogenic effectiveness of the composition.

A contemplated vaccine or inoculum advantageously also includes an adjuvant. Suitable adjuvants for vaccines and inocula of the present invention comprise those adjuvants that are capable of enhancing the antibody responses against B cell epitopes of the chimer, as well as adjuvants capable of enhancing cell mediated responses towards T cell epitopes contained in the chimer. Adjuvants are well known in the art °(see, for example, Vaccine Design—The Subunit and Adjuvant Approach, 1995, Pharmaceutical Biotechnology, Volume 6, Eds. Powell, M. F., and Newman, M. J., Plenum Press, New York and London, ISBN 0-306-44867-X).

Exemplary adjuvants include complete

Freund's adjuvant (CFA) that is not used in humans, incomplete Freund's adjuvant (IFA), squalene, squalane and alum [e.g., Alhydrogel™ (Superfos, Denmark)], which are materials well known in the art, and are available commercially from several sources.

Preferred adjuvants for use with immunogens of the present invention include aluminum or calcium salts (for example hydroxide or phosphate salts). A particularly preferred adjuvant for use herein is an aluminum hydroxide gel such as Alhydrogel™. For aluminum hydroxide gels, the chimer protein is admixed with the adjuvant so that between 50 to 800 micrograms of aluminum are present per dose, and preferably between 400 and 600 micrograms are present.

Another particularly preferred adjuvant for use with an immunogen of the present invention is an emulsion. A contemplated emulsion can be an oil-in-water emulsion or a water-in-oil emulsions. In addition to the immunogenic chimer protein, such emulsions comprise an oil phase of squalene, squalane, peanut oil or the like as are well-known, and a dispersing agent. Non-ionic dispersing agents are preferred and such materials include mono- and di-C₁₂-C₂₄-fatty acid esters of sorbitan and mannide such as sorbitan mono-stearate, sorbitan mono-oleate and mannide mono-oleate. An immunogen-containing emulsion is administered as an emulsion.

Preferably, such emulsions are water-in-oil emulsions that comprise squalene and mannide mono-oleate (Arlacel™ A), optionally with squalane, emulsified with the chimer protein in an aqueous phase. Well-known examples of such emulsions include Montanide™ ISA-720, and Montanide™ ISA 703 (Seppic, Castres, France), each of which is understood to contain both squalene and squalane, with squalene predominating in each, but to a lesser extent in Montanide™ ISA 703. Most preferably, Montanide™ ISA-720 is used, and a ratio of oil-to-water of 7:3 (w/w) is used. Other preferred oil-in-water emulsion adjuvants include those disclosed in WO 95/17210 and EP 0 399 843.

The use of small molecule adjuvants is also contemplated herein. One type of small molecule adjuvant useful herein is a 7-substituted-8-oxo- or 8-sulfo-guanosine derivative described in U.S. Pat. No. 4,539,205, No. 4,643,992, No. 5,011,828 and No. 5,093,318, whose disclosures are incorporated by reference. Of these materials, 7-allyl-8-oxoguanosine (loxoribine) is particularly preferred. That molecule has been shown to be particularly effective in inducing an antigen-(immunogen-) specific response.

Still further useful adjuvants include monophosphoryl lipid A (MPL) available from Corixa Corp. (see, U.S. Pat. No. 4,987,237), CPG available from Coley Pharmaceutical Group, QS21 available from Aquila Biopharmaceuticals, Inc., SBAS2 available from SKB, the so-called muramyl dipeptide analogues described in U.S. Pat. No. 4,767,842, and MF59 available from Chiron Corp. (see, U.S. Pat. No. 5,709,879 and No. 6,086,901).

More particularly, immunologically active saponin fractions having adjuvant activity derived from the bark of the South American tree Quillaja Saponaria Molina (e.g. Quil™ A) are also useful. Derivatives of Quil™ A, for example QS21 (an HPLC purified fraction derivative of Quil™ A), and the method of its production is disclosed in U.S. Pat. No. 5,057,540. In addition to QS21 (known as QA21), other fractions such as QA17 are also disclosed.

3-De-O-acylated monophosphoryl lipid A is a well-known adjuvant manufactured by Ribi Immunochem, Hamilton, Mont. The adjuvant contains three components extracted from bacteria, monophosphoryl lipid (MPL) A, trehalose dimycolate (TDM) and cell wall skeleton (CWS) (MPL+TDM+CWS) in a 2% squalene/Tween® 80 emulsion. This adjuvant can be prepared by the methods taught in GB 2122204B. A preferred form of 3-de-O-acylated monophosphoryl lipid A is in the form of an emulsion having a small particle size less than 0.2 μm in diameter (EP 0 689 454 B1).

The muramyl dipeptide adjuvants include N-acetyl-muramyl-L-threonyl-D-isoglutamine(thur-MDP), N-acetyl-nor-muramyl-L-alanyl-D-isoglutamine (CGP 11637, referred to as nor-MDP), and N-acetylmuramyl-L-alanyl-D-isoglutaminyl-L-alanine-2-(1′-2′-dipalmityol-sn-glycero-3-hydroxyphosphoryloxy)-ethylamin (CGP) 1983A, referred to as MTP-PE).

Preferred adjuvant mixtures include combinations of 3D-MPL and QS21 (EP 0 671 948 B1), oil-in-water emulsions comprising 3D-MPL and QS21 (WO 95/17210, PCT/EP98/05714), 3D-MPL formulated with other carriers (EP 0 689 454 B1), QS21 formulated in cholesterol-containing liposomes (WO 96/33739), or immunostimulatory oligonucleotides (WO 96/02555). Alternative adjuvants include those described in WO 99/52549 and non-particulate suspensions of polyoxyethylene ether (UK Patent Application No. 9807805.8).

Adjuvants are utilized in an adjuvant amount, which can vary with the adjuvant, mammal and recombinant HBc chimer immunogen. Typical amounts can vary from about 1 μg to about 1 mg per immunization. Those skilled in the art know that appropriate concentrations or amounts can be readily determined.

Inocula and vaccines are conventionally administered parenterally, by injection, for example, either subcutaneously or intramuscularly. Additional formulations that are suitable for other modes of administration include suppositories and, in some cases, oral formulation. The use of a nasal spray for inoculation is also contemplated as discussed in Neirynck et al. (October 1999) Nature Med., 5(10):1157-1163. For suppositories, traditional binders and carriers can include, for example, polyalkalene glycols or triglycerides; such suppositories may be formed from mixtures containing the active ingredient in the range of 0.5% to 10%, preferably 1-2%. Oral formulations include such normally employed excipients as, for example, pharmaceutical grades of mannitol, lactose, starch, magnesium stearate, sodium saccharine, cellulose, magnesium carbonate and the like.

An inoculum or vaccine composition takes the form of a solution, suspension, tablet, pill, capsule, sustained release formulation or powder, and contains an immunogenic effective amount of HBc chimer or HBc chimer conjugate, preferably as particles, as active ingredient. In a typical composition, an immunogenic effective amount of preferred HBc chimer or HBc chimer conjugate particles is about 1 μg to about 1 mg of active ingredient per dose, and more preferably about 5 μg to about 50 μg per dose, as noted before.

A vaccine is typically formulated for parenteral administration. Exemplary immunizations are carried out sub-cutaneously (SC) intra-muscularly (IM), intravenusly (IV), intraperitoneally (IP) or intra-dermally (ID).

The HBc chimer particles and HBc chimer particle conjugates can be formulated into the vaccine as neutral or salt forms. Pharmaceutically acceptable salts, include the acid addition salts (formed with the free amino groups of the protein or hapten) and are formed with inorganic acids such as, for example, hydrochloric or phosphoric acids, or such organic acids as acetic, oxalic, tartaric, mandelic, and the like. Salts formed with the free carboxyl groups can also be derived form inorganic bases such as, for example, sodium, potassium, ammonium, calcium, or ferric hydroxides, and such organic bases as isopropylamine, trimethylamine, 2-ethylamino ethanol, histidine, procaine, and the like.

In yet another embodiment, a vaccine or inoculum is contemplated in which a gene encoding a contemplated HBc chimer is transfected into suitably attenuated enteric bacteria such as S. typhi, S. typhimurium, S. typhimurium-E. coli hybrids or E. coli. Exemplary attenuated or avirulent S. typhi and S. typhimurium and S. typhimurium-E. coli hybrids are discussed in the citations provided before. These vaccines and inocula are particularly contemplated for use against diseases that infect or are transmitted via mucosa of the nose, the gut and reproductive tract such as influenza, yeasts such as Aspergiullus and Candida, viruses such as polio, moot-and-mouth disease, hepatitis A, and bacteria such as Cholera, Salmonella and E. coli and where a mucosal IgA response is desired in addition to or instead of an IgG systemic response.

The enteric bacteria can be freeze dried, mixed with dry pharmaceutically acceptable diluents, made into tablets or capsules for ingestion and administered to or taken by the host animal as are usual solid phase medications. In addition, aqueous preparations of these bacterial vaccines are adapted for use in mucosal immunization as by oral, nasal, rectal or vaginal administration.

Oral immunization using plant matter containing contemplated chimeric molecule particles can be achieved by simple ingestion of the transgenic plant tissue such as a root like a carrot or seed such as rice or corn. In this case, the water of the mouth or gastrointestinal tract provides the usually used aqueous medium used for immunization and the surrounding plant tissue provides the pharmaceutically acceptable diluent.

The inocula or vaccines are administered in a manner compatible with the dosage formulation, and in such amount as are therapeutically effective and immunogenic. The quantity to be administered depends on the subject to be treated, capacity of the subject's immune system to synthesize antibodies, and degree of protection desired. Precise amounts of active ingredient required to be administered depend on the judgment of the practitioner and are peculiar to each individual. However, suitable dosage ranges are of the order of tens of micrograms active ingredient per individual. Suitable regimes for initial administration and booster shots are also variable, but are typified by an initial administration followed in intervals (weeks or months) by a subsequent injection or other administration.

Once immunized, the mammal is maintained for a period of time sufficient for the recombinant HBc chimer immunogen to induce the production of a sufficient titer of antibodies that bind to an antigen of interest such as a sporozoite for a malarial vaccine. The maintenance time for the production of illustrative anti-sporozoite antibodies typically lasts for a period of about three to about twelve weeks, and can include a booster, second immunizing administration of the vaccine. A third immunization is also contemplated, if desired, at a time 24 weeks to five years after the first immunization. It is particularly contemplated that once a protective level titer of antibodies is attained, the vaccinated mammal is preferably maintained at or near that antibody titer by periodic booster immunizations administered at intervals of about 1 to about 5 years.

The production of anti-sporozoite or other antibodies is readily ascertained by obtaining a plasma or serum sample from the immunized mammal and assaying the antibodies therein for their ability to bind to an approriate antigen such as a synthetic circumsporozoite immunodominant antigen [e.g. the P. falciparum CS protein peptide (NANP)₅ used herein] in an ELISA assay as described hereinafter or by another immunoassay such as a Western blot as is well known in the art.

It is noted that the induced antibodies such as anti-CS antibodies can be isolated from the blood of an inoculated host mammal using well known techniques, and then reconstituted into a second vaccine for passive immunization as is also well known. Similar techniques are used for gamma-globulin immunizations of humans. For example, antiserum from one or a number of immunized hosts can be precipitated in aqueous ammonium sulfate (typically at 40-50 percent of saturation), and the precipitated antibodies purified chromatographically as by use of affinity chromatography in which (NANP)₅ is utilized as the antigen immobilized on the chromatographic column. Thus, for example, an inoculum can be used in a horse or sheep to induce antibody production against a malarial species for use in a passive immunization in yet another animal such as humans.

Another embodiment of the invention is a process for inducing antibodies, activated T cells or both in an animal host comprising the steps of inoculating said animal host with an inoculum. The inoculum used in the process comprises an immunogenic amount of a before-described HBc chimer particle or HBc chimer particle conjugate dissolved or dispersed in a pharmaceutically acceptable diluent. The animal host is maintained for a time sufficient for antibodies or activated T cells to be induced, as can be assayed by well-known techniques, which typically requires a time period of weeks to months, as is again well-known. A plurality of such immunizations is contemplated during this maintenance period.

The invention is illustrated by the following non-limiting examples.

### EXAMPLE 1

### B Cell Epitope-Containing Chimer Preparation

A. Preparation of Plasmid Vector pKK223-3N, a modified form of pKK223-3

Plasmid vector pKK223-3 (Pharmacia) was modified by the establishment of a unique NcoI restriction site to enable insertion of HBc genes as NcoI-HindIII restriction fragments and subsequent expression in E. coli host cells. To modify the pKK223-3 plasmid vector, a new SphI-HindIII fragment was prepared using the PCR primers pKK223-3/433-452-F and pKK223-NcoI-mod-R, and pKK223-3 as the template. This PCR fragment was cut with the restriction enzymes SphI and HindIII to provide a 467 bp fragment that was then ligated with a 4106 bp fragment of the pKK223-3 vector, to effectively replace the original 480 bp SphI-HindIII fragment. The resultant plasmid (pKK223-3N) is therefore 13 bp shorter than the parent plasmid and contains modified nucleotide sequence upstream of the introduced NcoI site (see FIG. 1 in which the dashes indicate the absent bases). The final plasmid, pKK223-3N, has a size of 4573 bp. Restriction sites in plasmid pKK223-3N are indicated in FIG. 1, and the nucleotide changes made to pKK223-3 to form plasmid pKK223-3N are indicated by an underline as shown below.

| | |
|---|---|
| pKK223-3/433-452-F | |
| GGTGCATGCAAGGAGATG | SEQ ID NO:65 |
| pKK223-NcoI-mod-R | |
| GCGAAGCTTCGGATCccatggTTTTTTCCTCCTTATG- | SEQ ID NO:66 |
| TGAAATTGTTATCCGCTC | |

B. Preparation of V1 and V2 Cloning Vectors

Modified HBc149 genes, able to accept the directional insertion of synthetic dsDNA fragments into the immunodominant loop region, were constructed using PCR. [The plasmid accepting inserts between amino acids E77 and D78 was named V1, whereas the plasmid accepting inserts between D78 and P79 was named V2.] The HBc149 gene was amplified in two halves using two PCR primer pairs, one of which amplifies the amino terminus, the other amplifies the carboxyl terminus. For V1, the products of the PCR reactions (N- and C-terminus) are both 246 bp fragments; for V2, the products are a 249 bp (N-terminus) and a 243 bp fragment (C-terminus).

The N-terminal fragments prepared were digested with NcoI and EcoRI, and the C-terminal fragments were digested with EcoRI and HindIII. The V1 and V2 fragments pairs were then ligated together at the common EcoRI overhangs. The resultant NcoI-HindIII fragments were then ligated into the pKK223-3N vector, which had been prepared by digestion with NcoI and HindIII.

To insert B cell epitopes into the V1 and V2 plasmids, the plasmids were digested with EcoRI and SacI restriction enzymes. Synthetic dsDNA fragments containing 5′ EcoRI and 3′ SacI overhangs were then inserted. In both cases, V1 and V2, glycine-isoleucine (EcoRI) and glutamic acid-leucine (SacI) amino acid pairs, coded for by the restriction sites, flank the inserted B cell epitopes. The inserted restriction sites are underlined in the primers below.

| | |
|---|---|
| V1 | |
| HBc149/NcoI-F | |
| 5′-TTGGGCCATGGACATCGACCCTTA | SEQ ID NO:67 |
| HBc-E77/EcoRI-R | |
| 5′-GCGGAATTCCTTCCAAATTAACACCCACC | SEQ ID NO:68 |
| HBc-D78/EcoRI-SacI-F | |
| 5′-CGCGAATTCAAAAAGAGCTCGATCCAGCGTCTA | SEQ ID NO:69 |
| GAGAC | |
| HBc149/HindIII-R | |
| 5′-CGCAAGCTTAAACAACAGTAGTCTCCGGAAG | SEQ ID NO:70 |
| V2 | |
| HBc149/NcoI-F | |
| 5′-TTGGGCCATGGACATCGACCCTTA | SEQ ID NO:67 |
| HBc-D78/EcoRI-R | |
| 5′-GCGGAATTCCATCTTCCAAATTAACACCCAC | SEQ ID NO:72 |
| HBc-P79/EcoRI-SacI-F | |
| 5′-CGCGAATTCAAAAAGAGCTCCCAGCGTCTAGAGA | SEQ ID NO:73 |
| CCTAG | |
| HBc149/HindIII-R | |
| 5′-CGCAAGCTTAAACAACAGTAGTCTCCGGAAG | SEQ ID NO:70 |

C. Preparation of V7 Cloning Vector

To enable the fusion of T cell epitopes to the C terminus of a HBc chimer, a new vector, V7, was constructed. Unique EcoRI and SacI restriction sites were inserted between valine-149 and the HindIII site to facilitate directional insertion of synthetic dsDNAs into EcoRI-HindIII (or EcoRI-SacI) restriction sites. The pair of PCR primers below was used to amplify the HBc 149 gene with a NcoI restriction site at the amino-terminus and EcoRI, SacI and HindIII sites at the carboxyl-terminus. The product of the PCR reaction (479 bp) was digested with NcoI/HindIII and cloned into pKK223-3N to form V7.

To insert T cell epitopes, the plasmid (V7) was digested EcoRI/HindIII (or EcoRI-SacI) and synthetic dsDNA fragments having EcoRI/HindIII (or EcoRI/SacI) overhangs, were ligated into V7. For all V7 constructs, the final amino acid of native HBc (valine-149) and the first amino acid of the inserted T cell epitope are separated by a glycine-isoleucine dipeptide sequence coded for by the nucleotides that form the EcoRI restriction site. For epitopes inserted at EcoRI/SacI, there are additional glutamic acid-leucine residues after the T cell epitope, prior to the termination codon, contributed by the SacI site. Restriction sites are again underlined in the primers shown.

| | |
|---|---|
| HBc149/NcoI-F | |
| 5′-TTGGGCCATGGACATCGACCCTTA | SEQ ID NO:67 |
| HBc149/SacI-EcoRI-H3-R | |
| 5′-CGCAAGCTTAGAGCTCTTGAATTCCAACAACAGT | SEQ ID NO:75 |
| AGTCTCCG | |

D. Preparation of V12 Expression Constructs

V12 vectors, which contain B cell epitopes between amino acids 78 and 79, as well as T cell epitopes downstream of valine-149, were constructed from V2 and V7 vectors. The carboxyl terminus of a V7 vector containing a AT cell epitope inserted at EcoRI/HindIII was amplified using two PCR primers (HBc-P79/SacI-F and pKK223-2/4515-32R) to provide a dsDNA fragment corresponding to amino acids 79-149 plus the T cell epitope, flanked with SacI and HindIII restriction sites.

The PCR products were cut with SacI and

HindIII and then cloned into the desired V2 vector prepared by cutting with the same two enzymes. The PCR primers shown are amenable for the amplification of the carboxyl terminus of all V7 genes, irrespective of the T cell epitope present after amino acid 149 of the HBc gene.

One exception to the generality of this approach was in the preparation of the V12 constructs containing the Pf-CS(C17A) mutation, which were prepared from existing V12 constructs. In this case, V12 constructs were amplified with HBc149/NcoI-F (SEQ ID NO:67) and the mis-match reverse PCR primer (SEQ ID NO: 145), which facilitated the C17A mutation. The resultant PCR product was digested with NcoI and HindIII and cloned back into pKK223-3N (previously cut with the same enzymes). Restriction sites are underlined.

| | |
|---|---|
| HBc-P79/SacI-F | |
| 5′-CGCGAGCTCCCAGCGTCTAGAGACCTAG | SEQ ID NO:76 |
| pKK223-2/4515-32R | |
| 5′-GTATCAGGCTGAAAATC | SEQ ID NO:77 |

E. P. falciparum CS-repeat B cell Epitopes Inserted into V2

For V2 and V7 constructs, synthetic dsDNA fragments coding for the B (V2) or T cell epitope (V7) of interest were inserted into EcoRI/SacI restriction sites. Synthetic dsDNA fragments, encoding B and T cell epitopes of interest, were prepared by mixing complementary single stranded DNA oligonucleotides at equimolar concentrations, heating to 95° C. for 5 minutes, and then cooling to room temperature at a rate of −1° C. per minute. This annealing reaction was performed in TE buffer. The double-stranded DNAs are shown below with the encoded epitope sequence shown above. The pound symbol, #, is used in some of the amino acid residue sequences that follow to indicate the presence of a stop codon.

| | |
|---|---|
| Pf1 | |
| I N A N P N A N P N A N P N A | |
| AATTAACGCTAATCCGAACGCTAATCCGAACGCTAATCCGAACGCTA | |
| TTGCGATTAGGCTTGCGATTAGGCTTGCGATTAGGCTTGCGAT | |
| N P E L | SEQ ID NO:78 |
| ATCCGGAGCT | SEQ ID NO:79 |
| TAGGCC | SEQ ID NO:80 |
| Pf3 | |
| I N A N P N V D P N A N P N A N P | |
| AATTAACGCTAATCCGAACGTTGACCCGAACGCTAATCCGAACGCTAATCCGA | |
| TTGCGATTAGGCTTGCAACTGGGCTTGCGATTAGGCTTGCGATTAGGCT | |
| N A N P N V D P N A N P E L | SEQ ID NO:81 |
| ACGCTAATCCGAACGTTGACCCGAACGCTAATCCGGAGCT | SEQ ID NO:82 |
| TGCGATTAGGCTTGCAACTGGGCTTGCGATTAGGCCTCGAGG | SEQ ID NO:83 |
| Pf3.1 | |
| I N A N P N V D P N A N P N A N P | |
| AATTAACGCGAATCCGAACGTGGATCCGAATGCCAACCCTAACGCCAACCC | |
| TTGCGCTTAGGCTTGCACCTAGGCTTACGGTTGGGATTGCGGTTGGG | |
| N A N P E L | SEQ ID NO:84 |
| AAATGCGAACCCAGAGCT | SEQ ID NO:85 |
| TTTACGCTTGGGTC | SEQ ID NO:86 |
| Pf3.2 | |
| I N A N P N A N P N A N P N V D P | |
| AATTAACGCGAATCCGAATGCCAACCCTAACGCCAACCCAAACGTGGATCCGA | |
| TTGCGCTTAGGCTTACGGTTGGGATTGCGGTTGGGTTTGCACCTAGGCT | |
| N A N P E L | SEQ ID NO:87 |
| ATGCGAACCCAGAGCT | SEQ ID NO:88 |
| TACGCTTGGGTC | SEQ ID NO:89 |
| Pf3.3 | |
| I N A N P N V D P N A N P N A N P | |
| AATTAACGCGAATCCGAACGTGGATCCAAATGCCAACCCTAACGCTAATCCAA | |
| TTGCGCTTAGGCTTGCACCTAGGTTTACGGTTGGGATTGCGATTAGGTT | |
| N A N P N V D P N A N P E L | SEQ ID NO:90 |
| ACGCCAACCCGAATGTTGACCCCAATGCCAATCCGGAGCT | SEQ ID NO:91 |
| TGCGGTTGGGCTTACAACTGGGGTTACGGTTAGGCC | SEQ ID NO:92 |
| Pf3.4 | |
| I N P N V D P N A N P N A N P N A | |
| AATTAATCCGAACGTGGATCCAAATGCCAACCCTAACGCTAATCCAAACGCCA | |
| TTAGGCTTGCACCTAGGTTTACGGTTGGGATTGCGATTAGGTTTGCGGT | |
| N P N V E L | SEQ ID NO:93 |
| ACCCGAATGTTGAGCT | SEQ ID NO:94 |
| TGGGCTTACAAC | SEQ ID NO:95 |
| Pf3.5 | |
| I N P N V D P N A N P N A N P N A | |
| AATTAATCCGAACGTGGATCCAAATGCCAACCCTAACGCTAATCCAAACGCCA | |
| TTAGGCTTGCACCTAGGTTTACGGTTGGGATTGCGATTAGGTTTGCGGT | |
| N P N V D P E L | SEQ ID NO:96 |
| ACCCGAATGTTGACCCTGAGCT | SEQ ID NO:97 |
| TGGGCTTACAACTGGGAC | SEQ ID NO:98 |
| Pf3.6 | |
| I N P N V D P N A N P N A N P N A | |
| AATTAATCCGAACGTGGATCCAAATGCCAACCCTAACGCTAATCCAAACGCCA | |
| TTAGGCTTGCACCTAGGTTTACGGTTGGGATTGCGATTAGGTTTGCGGT | |
| N P N V D P N A E L | SEQ ID NO:99 |
| ACCCGAATGTTGACCCTAATGCTGAGCT | SEQ ID NO:100 |
| TGGGCTTACAACTGGGATTACGAC | SEQ ID NO:101 |
| Pf3.7 | |
| I N V D P N A N P N A N P N A N P | |
| AATTAACGTGGATCCAAATGCCAACCCTAACGCTAATCCAAACGCCAACCCGA | |
| TTGCACCTAGGTTTACGGTTGGGATTGCGATTAGGTTTGCGGTTGGGCT | |
| N V E L | SEQ ID NO:102 |
| ATGTTGAGCT | SEQ ID NO:103 |
| TACAAC | SEQ ID NO:104 |
| Pf3.8 | |
| I N V D P N A N P N A N P N A N P | |
| AATTAACGTGGATCCAAATGCCAACCCTAACGCTAATCCAAACGCCAACCCGA | |
| TTGCACCTAGGTTTACGGTTGGGATTGCGATTAGGTTTGCGGTTGGGCT | |
| N V D P E L | SEQ ID NO:105 |
| ATGTTGACCCTGAGCT | SEQ ID NO:106 |
| TACAACTGGGAC | SEQ ID NO:107 |
| Pf3.9 | |
| I N V D P N A N P N A N P N A N P | |
| AATTAACGTGGATCCAAATGCCAACCCTAACGCTAATCCAAACGCCAACCCGA | |
| TTGCACCTAGGTTTACGGTTGGGATTGCGATTAGGTTTGCGGTTGGGCT | |
| N V D P N A E L | SEQ ID NO:108 |
| ATGTTGACCCTAATGCTGAGCT | SEQ ID NO:109 |
| TACAACTGGGATTACGAC | SEQ ID NO:110 |
| Pf3.10 | |
| I D P N A N P N A N P N A N P | |
| AATTGATCCAAATGCCAACCCTAACGCTAATCCAAACGCCAACC | |
| CTAGGTTTACGGTTGGGATTGCGATTAGGTTTGCGGTTGG | |
| N V E L | SEQ ID NO:111 |
| CGAATGTTGAGCT | SEQ ID NO:112 |
| GCTTACAAC | SEQ ID NO:113 |
| Pf3.11 | |
| I D P N A N P N A N P N A N P N V | |
| AATTGATCCAAATGCCAACCCTAACGCTAATCCAAACGCCAACCCGAATGTTG | |
| CTAGGTTTACGGTTGGGATTGCGATTAGGTTTGCGGTTGGGCTTACAAC | |
| D P E L | SEQ ID NO:114 |
| ACCCTGAGCT | SEQ ID NO:115 |
| TGGGAC | SEQ ID NO:116 |
| Pf3.12 | |
| I D P N A N P N A N P N A N P N V | |
| AATTGATCCAAATGCCAACCCTAACGCTAATCCAAACGCCAACCCGAATGTTG | |
| CTAGGTTTACGGTTGGGATTGCGATTAGGTTTGCGGTTGGGCTTACAAC | |
| D P N A E L | SEQ ID NO:117 |
| ACCCTAATGCCGAGCT | SEQ ID NO:118 |
| TGGGATTACGGC | SEQ ID NO:119 |

F. P. falciparum universal T cell epitope

| | |
|---|---|
| Pf-UTC (PF/CS326-345) | |
| I E Y L N K I Q N S L S T E W S P | |
| AATTGAATATCTGAACAAAATCCAGAACTCTCTGTCCACCGAATGGTCTCCGT | |
| CTTATAGACTTGTTTTAGGTCTTGAGAGACAGGTGGCTTACCAGAGGCA | |
| C S V T # # | SEQ ID NO:120 |
| GCTCCGTTACCTAGTA | SEQ ID NO:121 |
| CGAGGCAATGGATCATTCGA | SEQ ID NO:122 |

P. vivax CS-repeat B cell epitopes

| | |
|---|---|
| Pv-T1A | |
| I P A G D R A D G Q P A G D R A A | |
| AATTCCGGCTGGTGACCGTGCAGATGGCCAGCCAGCGGGTGACCGCGCTGCAG | |
| GGCCGACCACTGGCACGTCTACCGGTCGGTCGCCCACTGGCGCGACGTC | |
| G Q P A G E L | SEQ ID NO:123 |
| GCCAGCCGGCTGGCGAGCT | SEQ ID NO:124 |
| CGGTCGGCCGACCGC | SEQ ID NO:125 |
| Pv-T1B | |
| I D R A A G Q P A G D R A D G Q P | |
| AATTGACAGAGCAGCCGGACAACCAGCAGGCGATCGAGCAGACGGACAGCCCG | |
| CTGTCTCGTCGGCCTGTTGGTCGTCCGCTAGCTCGTCTGCCTGTCGGGC | |
| A G E L | SEQ ID NO:126 |
| CAGGGGAGCT | SEQ ID NO:127 |
| GTCCCC | SEQ ID NO:128 |
| Pv-T2A | |
| I A N G A G N Q P G A N G A G D Q | |
| AATTGCGAACGGCGCCGGTAATCAGCCGGGGGCAAACGGCGCGGGTGATCAAC | |
| CGCTTGCCGCGGCCATTAGTCGGCCCCCGTTTGCCGCGCCCACTAGTTG | |
| P G E L | SEQ ID NO:129 |
| CAGGGGAGCT | SEQ ID NO:130 |
| GTCCCC | SEQ ID NO:131 |
| Pv-T2B | |
| I A N G A D N Q P G A N G A D D Q | |
| AATTGCGAACGGCGCCGATAATCAGCCGGGTGCAAACGGGGCGGATGACCAAC | |
| CGCTTGCCGCGGCTATTAGTCGGCCCACGTTTGCCCCGCCTACTGGTTG | |
| P G E L | SEQ ID NO:132 |
| CAGGCGAGCT | SEQ ID NO:133 |
| GTCCGC | SEQ ID NO:134 |
| Pv-T2C | |
| I A N G A G N Q P G A N G A G D Q | |
| AATTGCGAACGGCGCCGGTAATCAGCCGGGAGCAAACGGCGCGGGGGATCAAC | |
| CGCTTGCCGCGGCCATTAGTCGGCCCTCGTTTGCCGCGCCCCCTAGTTG | |
| P G A N G A D N Q P G A N G A D D | |
| CAGGCGCCAATGGTGCAGACAACCAGCCTGGGGCGAATGGAGCCGATGACC | |
| GTCCGCGGTTACCACGTCTGTTGGTCGGACCCCGCTTACCTCGGCTACTGG | |
| Q P G E L | SEQ ID NO:135 |
| AACCCGGCGAGCT | SEQ ID NO:136 |
| TTGGGCCGC | SEQ ID NO:137 |
| PV-T3 | |
| I A P G A N Q E G G A A A P G A N | |
| AATTGCGCCGGGCGCCAACCAGGAAGGTGGGGCTGCAGCGCCAGGAGCCAATC | |
| CGCGGCCCGCGGTTGGTCCTTCCACCCCGACGTCGCGGTCCTCGGTTAG | |
| Q E G G A A E L | SEQ ID NO:138 |
| AAGAAGGCGGTGCAGCGGAGCT | SEQ ID NO:139 |
| TTCTTCCGCCACGTCGCC | SEQ ID NO:140 |

Example 2: P. vivax universal T cell epitope

Pv-UTC
I E Y L D K V R A T V G T E W T P
AATTGAATATCTGGATAAAGTGCGTGCGACCGTTGGCACGGAATGGACTCCGT
CTTATAGACCTATTTCACGCACGCTGGCAACCGTGCCTTACCTGAGGCA

| | |
|---|---|
| C S V T # # | SEQ ID NO:141 |
| GCAGCGTGACCTAATA | SEQ ID NO:142 |
| CGTCGCACTGGATTATTCGA | SEQ ID NO:143 |

A. PCR Primers for Site-Directed Mutagenesis

| | |
|---|---|
| Pf-CS(C17A)-R | SEQ ID NO:144 |
| # # T V S A P S W E T S | |
| GCCAAGCTTACTAGGTAACGGAGGCCGGAGACCATTCGGTGG | |
| HindIII | SEQ ID NO:145 |

B. PCR Primers for Truncation and Cysteine Addition at C-terminus

To modify the C-terminus of HBc chimer genes, either via the addition of cysteine residues or varying the length of the HBc gene, PCR reactions were performed using HBc149 as template with the HBc/NcoI-F primer and a reverse primer (e.g. HBc149+C/HindIII-R) that directed the desired modification of the C-terminus. PCR products were digested with NcoI and HindIII and cloned into pKK223-3N at the same restriction sites.

| | |
|---|---|
| HBc149/NcoI-F | SEQ ID NO:245 |
| M D I D P Y | |
| 5′-TTGGGCCATGGACATCGACCCTTA | SEQ ID NO:67 |
| HBc149 + C/HindIII-R | SEQ ID NO:147 |
| # C V V T T E P L | |
| 5′-CGCAAGCTTACTAGCAAACAACAGTAGTCTCCGGAAG | |
| HindIII | SEQ ID NO:148 |
| HBC144/HindIII-R | SEQ ID NO:149 |
| # P L T S L I P | |
| CGCAAGCTTACGGAAGTGTTGATAGGATAGGG | SEQ ID NO:150 |
| HBc142/HindIII-R | SEQ ID NO:151 |
| # T S L I P A N P | |
| CGCAAGCTTATGTTGATAGGATAGGGGCATTTGG | SEQ ID NO:152 |
| HBc140/HindIII-R | SEQ ID NO:153 |
| # L I P A N P P | |
| CGCAAGCTTATAGGATAGGGGCATTTGGTGG | SEQ ID NO:154 |
| HBc139/HindIII-R | SEQ ID NO:155 |
| # I P A N P P | |
| GCGAAGCTTAGATAGGGGCATTTGGTGG | SEQ ID NO:156 |
| HBc138/HindIII-R | SEQ ID NO:157 |
| # P A N P P R | |
| CGCAAGCTTAAGGGGCATTTGGTGGTCT | SEQ ID NO:158 |
| HBc138 + C/HindIII-R | SEQ ID NO:159 |
| # C P A N P P R | |
| GCGAAGCTTAGCAAGGGGCATTTGGTGGTCT | SEQ ID NO:160 |
| HBc137/HindIII-R | SEQ ID NO:161 |
| # A N P P R Y A | |
| GCGAAGCTTAGGCATTTGGTGGTCTATAGC | SEQ ID NO:162 |
| HBc137 + C/HindIII-R | SEQ ID NO:163 |
| # C A N P P R Y A | |
| GCGAAGCTTAGCAGGCATTTGGTGGTCTATAA | SEQ ID NO:164 |
| HBc136/HindIII-R | SEQ ID NO:165 |
| # N P P R Y A P | |
| CGCAAGCTTAATTTGGTGGTCTATAAGCTGG | SEQ ID NO:166 |

### EXAMPLE 3

### Assay Procedures

A. Antigenicity

1. Particle ELISA

Purified particles were diluted to a concentration of 10 μg/mL in coating buffer (50 mM sodium bicarbonate, pH 9.6) and coated onto the wells of ELISA strips (50 μL/well). The ELISA strips were incubated at room temperature overnight (about 18 hours). Next morning the wells were washed with ELISA wash buffer [phosphate buffered saline (PBS), pH 7.4, 0.05% Tween®-20] and blocked with 3% BSA in PBS for 1 hour (75 μL/well). ELISA strips were stored, dry, at −20° C. until needed.

To determine the antigenicity of particles, antisera were diluted using 1% BSA in PBS and 50 μL/well added to antigen-coated ELISA wells. Sera were incubated for 1 hour, washed with ELISA wash buffer and probed using an anti-mouse(IgG)-HRP (The Binding Site, San Diego, Calif.; HRP=horseradish peroxidase) conjugate (50 μL/well) or other appropriate antibody for 30 minutes. After washing with ELISA wash buffer the reaction was visualized by the addition of TM blue substrate (50 μL/well). After 10 minutes, the reaction was stopped by the addition of 1N H₂SO₄ (100 μL/well) and read on an ELISA plate reader set at 450 nm.

2. Synthetic Peptide ELISA

A 20 amino acid residue synthetic peptide (NANP)₅ was diluted to a concentration of 2 μg/mL in coating buffer (50 mM sodium bicarbonate, pH 9.6) and coated onto the wells of ELISA strips (50 μL/well). Peptides were dried onto the wells by incubating overnight (about 18 hours), in a hood with the exhaust on. Next morning, the wells were washed with ELISA wash buffer (phosphate buffered saline, pH 7.4, 0.05% Tween®-20) and blocked with 3% BSA in PBS (75 μL/well) for 1 hour. ELISA strips were stored, dry, at −20° C. until needed.

To determine antibody antigenicity of particles, antisera (monoclonal or polyclonal) were diluted using 1% BSA in PBS, and 50 μL/well added to antigen-coated ELISA wells. Sera were incubated for 1 hour, washed with ELISA wash buffer, and probed using an anti-mouse(IgG)-HRP conjugate (as above at 50 μL/well) or other appropriate antibody for 30 minutes, washed again with ELISA wash buffer, and then visualized by the addition of TM blue substrate (50 μL/well). After 10 minutes, the reaction was stopped by the addition of 1N H₂SO₄ (100 μL/well) and read on an ELISA plate reader set at 450 nm.

B. Immunogenicity of Particles

To assay the immunogenicity of particles, mice were immunized, IP, with 20 μg of particles in Freund's complete adjuvant, and then boosted at 4 weeks with 10 μg in Freund's incomplete adjuvant. Mice were bled at 2, 4, 6, and 8 weeks.

C. Sporozoite IFA

Indirect immunofluorescence assay (IFA) was carried out using glutaraldehyde-fixed P. falciparum sporozoites and FITC-labeled anti-mouse IgG (gamma-chain specific) (Kirkegaard and Perry, Gaithersburg, Md.) to detect bound antibody [Munesinghe et al., Eur. J. Immunol. 1991, 21, 3015-3020]. Sporozoites used were dissected from the salivary glands of Anopheles mosquitoes infected by feeding on P. falciparum (NF54 isolate) gametocytes derived from in vitro cultures.

### EXAMPLE 4

### Expression of Recombinant Chimer HBc Particles

A. Effect of Insertion Position on Immunogenicity

Antibody titers (1/reciprocal dilution) were measured for mice immunized with HBc particles containing the P. f-CS B cell epitope (NANP)₄, inserted either between amino acids E77/D78 (SEQ ID NOs:260 and 261) or D78/P79 (SEQ ID NOs: 259 and 260), or by using a loop replacement approach (CS-2) [discussed in Schodel et al., (1994) J. Exp. Med., 180:1037-1046, using complete Freund's adjuvant]. Mice were immunized with a single 20 μg dose, IP, with adjuvant as noted before, and antibody titers determined in an ELISA using immobilized (NANP)₅ synthetic peptide. The results of those studies are shown in Table 1, below.

TABLE 1

| Time | CS-2* | E77/D78 (V1) | D78/P79 (V2) |
|---|---|---|---|
| 2 weeks | 0 | 2,560 | 2,560 |
| 4 weeks | 640 | 2,560 | 40,960 |

*Schodel et al., (1994) J. Exp. Med., 180: 1037-1046.

Another comparison was made of insertion position of the NANP CS-repeat epitope on immunogenicity, using BALB/c mice. Antibody titers induced by the CS-2 particle of Schodel et al. were compared to titers achieved using the same (NANP)₄ B cell epitope, inserted between HBc positions 78 and 79, and using the above V2.Pf1 particles as immunogen. Sera were analyzed 4 weeks after primary (1°) and 2 weeks after booster (2°) immunization, and the results are shown in Table 2, below.

TABLE 2

| Chimer | Primary | Booster |
|---|---|---|
| CS-2 | 0 | 640* |
| V2.Pf1 | 10,240 | 655,360 |

*Schodel et al., (1994) J. Exp. Med., 180: 1037-1046

A similar comparison of insertion position of the NANP CS-repeat epitope on immunogenicity was made using B10.S mice, and the results are shown in Table 3.

TABLE 3

| Chimer | Primary | Booster |
|---|---|---|
| CS-2 | 640* | 20,480* |
| V2.Pf1 | 163,840 | 655,360 |

*Schodel et al., (1994) J. Exp. Med., 180: 1037-1046

The effect on the immunogenicity of HBc chimer particles (ELISA, F1 mice) that include the minor B cell epitope, NANPNVDP (SEQ ID NO:167), along with a repeated NANP sequence was examined. A HBc chimer was expressed that contained the sequence NANPNVDP(NANP)₃NVDP (SEQ ID NO:21; V12.Pf3) inserted between HBc positions 78 and 79. The resulting ELISA data were compared to titers obtained using the tetrameric repeat (NANP)₄ B cell epitope (V12.Pf1) or the dimer of the minor B cell epitope at the same position (V12.Pf7). Each of these three chimers contained a Domain IV that included the HBc sequence from position 141 through 149, bonded to the P. falciparum universal T cell epitope as the C-terminal sequence. The results of these studies using primary and booster immunizations as discussed before and using adjuvants, are shown below in Table 4.

TABLE 4

| Chimer | Primary | Booster |
|---|---|---|
| V12.Pf1 | 163,840 | 655,360 |
| V12.Pf3 | 2,621,440 | 10,485,760 |
| V12.Pf7 | 2,560 | |

The observed greater than 20-fold increase in immunogenicity by including the ‘minor’ repeat epitope was quite unexpected. Because V12.Pf3 was not well expressed by E. coli, variants of the Pf3 epitope NANPNVDP(NANP)₃NVDP (SEQ ID NO:21) were constructed that had similar antigenicity to Pf3, but with increased expression levels, as shown below. Only constructs 3.1 and 3.2 were assayed for immunogenicity.

Relative expression levels of recombinant chimer HBc/P. falciparum particles and antigenicities for monoclonal antibodies specific for the CS epitopes (NANP)₄ and (NANPNVDP) are shown in Table 5 below. Relative expression levels are as follows; ****=75-125 mg/L; ***=50-75 mg/L; **=25-50 mg/L. Antigenicity was determined by end point titer dilutions for the monoclonal antibodies [MoAb 2A10 for (NANP)₄; MoAb 2B6D8 for NANPNVDP; and P. vivax Rpt. MoAb 2F2 provided by E. Nardin of New York University Medical Center]. The data were normalized such that the lowest titer is expressed as 1. For example, V12.Pf3 was 165 fold more antigenic than V12.Pf3.10 for the (NANP)₄-specific monoclonal, and 26-fold more antigenic than V12.Pf3.2 for the NANPNVDP-specific monoclonal antibody. N.D.=no detectable antibody binding. [Note: V12.Pf3.7 was not expressed due to a mutation in the expression vector; it was not examined further because similar constructs were not antigenic and re-cloning was therefore not a worthwhile endeavor.]

TABLE 5

| | P. falciparum B Cell | Relative | Antigenicity | |
|---|---|---|---|---|
| Name | Epitope | Expression | (NANP)₄ | NANPNVDP |

| | | | | |
|---|---|---|---|---|
| V12.Pf1 | (NANP)₄ | **** | 33 | ND |
| | SEQ ID NO:1 | | | |
| V12.Pf3 | NANPNVDP(NANP)₃NVDP | ** | 165 | 31 |
| | SEQ ID NO:21 | | | |
| V12 Pf3.1 | NANPNVDP(NANP)₃ | **** | 33 | 31 |
| | SEQ ID NO:22 | | | |
| V12.Pf3.2 | (NANP)₃NVDPNANP | *** | 33 | 1.2 |
| | SEQ ID NO:23 | | | |
| V12.Pf3.3 | NANPNVDP(NANP)₃ | ** | 5 | 1 |
| | NVDPNANP | | | |
| | SEQ ID NO:24 | | | |
| V12.PF3.4 | NPNVDP(NANP)₃NV | **** | 5 | 5 |
| | SEQ ID NO:25 | | | |
| V12.PF3.5 | NPNVDP(NANP)₃NVDP | **** | 5 | 5 |
| | SEQ ID NO:26 | | | |
| V12.PF3.6 | NPNVDP(NANP)₃ | **** | 5 | 5 |
| | NVDPNA | | | |
| | SEQ ID NO:27 | | | |
| V12.PF3.7 | NVDP(NANP)₃NV | — | — | — |
| | SEQ ID NO:28 | | | |
| V12.PF3.8 | NVDP(NANP)₃NVDP | **** | 5 | 1 |
| | SEQ ID NO:29 | | | |
| V12.PF3.9 | NVDP(NANP)₃NVDPNA | *** | 5 | ND |
| | SEQ ID NO:30 | | | |
| V12.PF3.10 | DP(NANP)₃NV | **** | 1 | ND |
| | SEQ ID NO:31 | | | |
| V12.PF3.11 | DP(NANP)₃NVDP | **** | 5 | ND |
| | SEQ ID NO:32 | | | |
| V12.PF3.12 | DP(NANP)₃NVDPNA | *** | 5 | ND |
| | SEQ ID NO:33 | | | |

Immunogenicity of selected HBc chimer particles containing variants of the Pf3 epitope were assayed as described above. Sera were analyzed by ELISA 4 weeks after primary (1°) and 4 weeks after booster (2°) immunizations. The data obtained are shown in Table 6, below, in which the “Name” of the chimer and the corresponding sequence of the B cell immunogen are as illustrated above.

TABLE 6

| NAME | PRIMARY | SECONDARY |
|---|---|---|
| V12.Pf1 | 40,960 | 655,360 |
| V12.Pf3 | 2,621,440 | 10,485,760 |
| V12.Pf3.1 | 2,621,440 | 10,485,760 |
| V12.Pf3.2 | 2,621,440 | 2,621,440 |

Surprisingly, a version that contained one copy of the NANPNVDP repeat (V12.Pf3.1) was as immunogenic (and expressed better) as a version containing 2 copies (V12.Pf3), despite being 5-fold less antigenic for the NANP monoclonal antibody.

B. Expression Failures

Several additional epitopes have been attempted to be placed into the HBc loop (Domain II) between positions 78 and 79 (as in V2.Pf1), and have failed to be expressed for reasons unknown. Table 7, below, enumerates those epitopes that have failed to express when inserted between D78 and P79 (V2) in a HBc chimer.

TABLE 7

| | Source of | Epitope |
|---|---|---|
| Designation | Epitope | (single letter) |
| V2.FGF-1 | Human FGF-1 | NYKKPK |
| (N7-K12) | | SEQ ID NO:168 |
| V2.FGF-1 | Human FGF-1 | KRGPRTH |
| (K118-H124) | | SEQ ID NO:169 |
| V2.Arom-479 | P450 Aromatase | LHPDETKNMLEMIFTPRNSDR |
| | | SEQ ID NO:170 |
| V2.HIV3.1 | HIV-1 (gp120) | RIKQI |
| | | SEQ ID NO:171 |
| V2.HIV4.1 | HIV-1 (gp120) | RIKQIGMPGGK |
| | | SEQ ID NO:172 |
| V2.HIV5.1 | HIV-1 (gp41) | LLELDKWASL |
| | | SEQ ID NO:173 |
| V2.HIV6.1 | HIV-1 (gp41) | EQELLELDKWASLW |
| | | SEQ ID NO:174 |
| V2 HIV9.1 | HIV-1 (gp41) | VQQQNNLLRAIEAQQHLL- |
| | | QLTVWGIKQLQARIL |
| | | SEQ ID NO:175 |
| V2.HIV10.1 | HIV-1 (gp41) | HLLQLTVWGIKQLQAR |
| | | SEQ ID NO:176 |
| V2.HIV12.1 | HIV-1 (gp41) | YTHIIYSLIEQSQNQQEK- |
| | | NEQELLALDKWASLWNWF |
| | | SEQ ID NO:177 |
| V2.HIV13.1 | HIV-1 (gp41) | YTHIIYSLIEQSQN- |
| | | QQEKNEQELLEL |
| | | SEQ ID NO:178 |
| V2.1A2 | Human P450-1A2 | GRERRPRLSDRPQLPYLEA |
| (351-370) | | SEQ ID NO:179 |
| V2.2D6 | Human P450-2D6 | REQRRFSVSTLRNLGLGKKS |
| (129-148) | | SEQ ID NO:180 |
| V2.Py-B1 | P. yoelii | PNKLPRSTAVVHQLKRKH |
| | (TRAP) | SEQ ID NO:181 |
| V2.Py-B3 | P. yoelii | TAVVHQLKRKH |
| | (TRAP) | SEQ ID NO:182 |
| V2.Pv-T1A | P. vivax | PAGDRADGQPAGDRAAAGQPAG |
| | | SEQ ID NO:183 |
| V2.ALV1.2 | ALV-J | NQSWTMVSPINV |
| | | SEQ ID NO:184 |
| V2.ALV1.2 | ALV-J | MIKNGTKRTAVTFGSV |
| | | SEQ ID NO:185 |
| V2.FMDV | FMDV | PNLRGDLQVLAQKVARTLP |
| (142-160) | | SEQ ID NO:186 |
| V2.FMDV | FMDV | RYNRNAVPNLRGDL- |
| (135-160) | | QVLAQKVARTLP |
| | | SEQ ID NO:187 |

### EXAMPLE 5

### Determination of 280/260 Absorbance Ratios

Protein samples were diluted to a concentration of between 0.1 and 0.3 mg/mL using phosphate buffered saline (PBS), pH 7.4. The spectrophotometer was blanked, using PBS, and the absorbance of the protein sample measured at wavelengths of 260 nm and 280 nm. The absorbance value determined for a sample at 280 nm was then divided by the absorbance value determined for the same sample at 260 nm to achieve the 280/260 absorbance ratio for a given sample. The ratios obtained for several samples, including native particles (HBc 183), HBc particles truncated after residue position 149 (HBc 149), and several HBc chimers that are identified elsewhere herein, are shown below in Table 8.

TABLE 8

| | 280/260 |
|---|---|
| Particle | Absorbance Ratio |
| HBc183 | 0.84 |
| HBc149 | 1.59 |
| V2.PF1 | 1.64 |
| V2.PF1 + C150 | 1.5 |
| V2.PF1 + | 1.54 |
| Pf/CS-UTC | |
| V2.PF1 + | 1.42 |
| Pf/CS-UTC (C17A) | |

### EXAMPLE 6

### Cysteine at the C-Terminus of Truncated HBc Particle

A. Addition of a Cysteine Residue to the C-terminus of Hybrid HBc Particles

Using the polymerase chain reaction (PCR), genes expressing hybrid HBc particles can be easily mutated to introduce a cysteine or cysteine-containing peptide to the C-terminus of HBc. For example, a PCR oligonucleotide primer of SEQ ID NO:148 can be used, in concert with a suitable second primer, to amplify a hybrid HBc gene and incorporate a cysteine codon between codon V149 and the stop codon.

Hepatitis B core particles can be truncated from 183 (or 185, depending on viral subtype) to 140 and retain the ability to assemble into particulate virus-like particles. Many groups have used particles truncated to amino acid 149 because amino acid 150 represents the first arginine residue of the arginine-rich C-terminal domain.

To assess the ability of a single cysteine residue to stabilize HBc particles, a codon for a cysteine residue was inserted using techniques described before between the codon for HBc amino acid residue V149 and the termination codon of a chimer HBc molecule that contained the (NANP)₄ malarial B cell epitope inserted between residues 78 and 79 (referred to herein as V2.Pf1) to form the chimeric molecule and particle referred to as V2.Pf1+C (HBc149C). The thermal stability (at 37° C.) of this chimer particle (V2.Pf1+C; SEQ ID NOs: 264 and 265) as compared to a similar chimer particle lacking the inserted cysteine (V2.Pf1) was found to be dramatically increased, as is seen in FIG. 3.

It is noted that vectors and expression products that are prepared by addition of a cysteine to the C-terminus of a V2 construct are sometimes referred to herein as V16 vectors or expression products.

As can readily be seen in FIG. 3, the two particles started out similarly. However, after fourteen days at 37° C., the cysteine-containing particle exhibited fewer bands on the SDS gel, indicating enhanced stability as compared to the particle lacking the added Cys residue.

B. Thermal Stability Protocol

Purified particles were diluted to a concentration of 1 mg/mL using 50 mM NaPO₄, pH 6.8 and sodium azide was added to a final concentration of 0.02% to prevent bacterial growth. Particles were incubated at 37° C. and aliquots were taken at the time points indicated in the drawing description. Samples were mixed with SDS-PAGE sample buffer (reducing) and run on 15% SDS-PAGE gels. Gels were stained using Coomassie Blue, and then analyzed.

### EXAMPLE 7

### Cysteine at the C-terminus of a Peptide Fused to the C-terminus of HBc

To further investigate whether terminal cysteine residues could elicit stabilizing effects at positions other than 150, a Th epitope from the hepatitis B core protein (amino acid residues 74-87) was fused to the C-terminus of HBc containing a malarial epitope in the immunodominant loop. This Th epitope does not contain a cysteine residue, so a Cys residue was added at the C-terminus (underlined “C”). The control was the same epitope lacking the cysteine. These particles were made by combining V2.Pf1 with V7.HBc74-87 (and V7.HBc74-87+C). The V7 construct was PCR amplified with the HBc-P79/SacI-F primer (SEQ ID NO: 76) and pKK223-2/4515-32-R (SEQ ID NO: 77). The product was cut with SacI and HindIII, and the SacI/HindIII fragment was ligated into V2.Pf1 cut with the same enzymes.

Table 9, below, shows the amino acid sequences of C-terminal fusions HBc(74-87) and HBc(74-87)+C, relative to the native sequence that occurs in the wild type HBc protein, as well as the and the HBc149+C particle. “Cys shift” is the position of the introduced cysteine relative to its location in the wild type protein, where it is the last residue (position 183).

TABLE 9

| | | | | | |
|---|---|---|---|---|---|
| | | | | Cys | |
| | | | | Po- | |
| | | | | si- | Cys |
| Source | Sequence | PI | Length | tion | Shift |

| | | | | | |
|---|---|---|---|---|---|
| Native | RRRGRSPRRRT- | 12.74 | 34 | 34 | Zero |
| | PSPRRRRSQSP- | | | | |
| | RRRRSQSRESQC | | | | |
| | SEQ ID NO:189 | | | | |
| HBc(74-87) | GIVNLEDPAS- | 3.78 | 16 | N/A | N/A |
| | RDLVVS | | | | |
| | SEQ ID NO:190 | | | | |
| HBc(74-87)+C | GIVNLEDPAS- | 3.78 | 16 | 16 | −17 |
| | RDLVVSC | | | | |
| | SEQ ID NO:191 | | | | |
| HBc-149+C | C | N/A | 1 | 1 | −33 |

### EXAMPLE 8

### Cysteine Located Within a Peptide Fused to the C-terminus of an HBc Hybrid

Studies were conducted to determine if there were an absolute requirement for a cysteine residue to be the final amino acid of the HBc gene (as it is in wild type HBc) or if a cysteine could function internally in an introduced C-terminal sequence.

A peptide corresponding to a 20-residue universal T cell epitope, derived from the CS protein of the malarial parasite Plasmodium falciparum, which contains a cysteine at position 17 of the peptide or 342 of the CS protein, [Calvo-Calle et al., J. Immunol., (1997) 159(3):p. 1362-1373], was fused to the C-terminus of a HBc chimer (V2.Pf1; SEQ ID NOs: 266 and 267). This chimer contains the HBc sequence from position 1 through position 149, with the P. falciparum B cell epitope (NANP)₄ inserted between amino acid residues 78 and 79. Domain I of this HBc construct thus contained residues 1-75; Domain II contained residues 76-85 with the (NANP)₄ epitope inserted between residues 78 and 79 (along with four residues comprising the restriction sites); Domain III contained residues 86-135; and Domain IV contained residues 136-149 plus the 20-residue P. falciparum T cell epitope and two residues from the EcoRI cloning site (GI).

This fused C-terminal peptide is 20 amino acid residues long (12 or 14 amino acids shorter than the wild type sequence, depending on virus subtype) and has a predicted pI value more than 8 pH units lower than the wild type sequence. To minimize potential stabilizing effects that may be contributed by amino acids other than the cysteine, a (similar) control construct was made, having an alanine instead of a cysteine at position 17 (see Table 10, below).

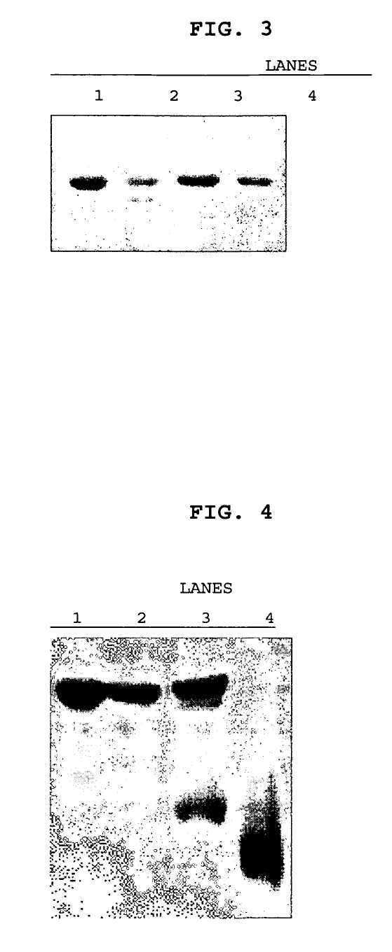

To enable simple assessment of the stabilizing effects of this sequence, the peptides were fused to the C-terminus of a particle previously shown to degrade readily at 37° C. (V2.Pf1) to form the HBc chimers denominated V2.Pf1+Pf/CS-UTC and V2.Pf1+Pf/CS-UTC(C17A), respectively. The results of a thermal stability study over a 28 day time period (as discussed previously) are shown in FIG. 4.

The results of this study showed that the presence of the cysteine in the T cell epitope derived from the CS protein of P. falciparum was needed for particle stability in the time period studied, and that there was no absolute requirement that that cysteine be at the C-terminus of the epitope. The table below shows the amino acid sequences of C-terminal fusions with a cysteine or alanine at position 17, relative to the native sequence, which occurs in the wild type HBc protein.

TABLE 10

| | | | | | |
|---|---|---|---|---|---|
| | | | | Cys | |
| | | | | Posi- | Cys |
| Source | Sequence | pI | Length | tion | Shift |

| | | | | | |
|---|---|---|---|---|---|
| Native | RRRGRSPRRRT- | 12.74 | 34 | 34 | zero |
| | PSPRRRRSQSP- | | | | |
| | RRRRSQSRESQC | | | | |
| | SEQ ID NO:189 | | | | |
| Pf/CS-UTC | (GI)EYLNKIQNS- | 4.44 | 20 | 17 | −15 |
| | LSTEWSPCSVT | | | | |
| | SEQ ID NO:2 | | | | |
| Pf/CS- | (GI)EYLNKIQNS- | 4.44 | 20 | N/A | N/A |
| UTC(C17A) | LSTEWSPASVT | | | | |
| | SEQ ID NO:192 | | | | |

(GI) = residues added from cloning site.

### EXAMPLE 9

### P. Vivax HBc Chimers

Following the work discussed before on HBc chimers containing P. falciparum B cell and T cell immunogens, similar work was carried out using sequences from the P. vivax CS protein. Exemplary constructs are illustrated below in Table 11.

TABLE 11

| | Malarial B Cell | |
|---|---|---|
| P. vivax | Immunogen | CS-UTC |
| Immunogen Type | (Between D78/P79) | (After V149) |
| Type-I | (DRA(A/D)GQPAG) | YLDKVRATVGTEWTPCSVT |
| | SEQ ID NO:193 | SEQ ID NO:196 |
| Type-II | (ANGA(G/D) (N/D)QPG) | YLDKVRATVGTEWTPCSVT |
| | SEQ ID NO:194 | SEQ ID NO:196 |
| Type-III | (APGANQEGGAA) | YLDKVRATVGTEWTPCSVT |
| (‘Vivax-like’) | SEQ ID NO:195 | SEQ ID NO:196 |

To address the variability of the repeats, the following variant epitopes were used for insertion into HBc between amino acids 78 and 79:

1. Type-I CS-Repeat

| | |
|---|---|
| PAGDRADGQPAGDRAAGQPAG (P. vivax-type 1A)--. | SEQ ID NO: 197 |
| This form of the epitope failed to make a | |
| particle. | |

DRAAGQPAGDRADGQPAG (P. vivax-type 1B)-SEQ ID NO: 198. This form of the epitope, containing flanking dipeptide cloning site remnants, successfully made a particle and is referred to as V2.PV-TIB. An immunogen for P. vivax-type I has been successfully cloned, expressed, purified, and its immunogenicity tested in mice. The results of that mouse study are shown in Table 12, hereinafter.

2. Type-II CS-Repeat

For type-II, this work is complicated by the existence of four different forms of the type-II epitope. These forms contain either G or D at position 5, and either N or D at position 6 [Qari et al., Mol. Biochem. Parasitol., (1992) 55(1-2):p. 105-113]. Hence, there are 4 different possible repeat sequences (GN, GD, DN, and DD) needed to maximize the possibility of success. The first, and preferred approach, is to prepare a single hybrid particle containing all four repeats, as shown below by underlines. This approach was successfully employed to address the variability in the type-I repeat. Each of these constructs contains flanking dipeptide cloning site remnants.

| | |
|---|---|
| ANGAGNQPGANGAGDQPGANGADNQPGANGADDQPG | SEQ ID NO: 199 |
| (P. vivax-type II-GN/GD/DN/DD). | |

The above sequence has been cloned, expressed, and purified as a HBc chimer with no modification to the C-terminus.

The second approach was to prepare two hybrid particles, whereby each particle contained two of the variant epitopes (see below). This approach is less preferable because it requires either the use of a more complex expression system to direct the production of ‘mixed’ particles during expression, or the mixing of type-II particles following manufacture.

| | |
|---|---|
| ANGAGNQPGANGAGDQPG (P. vivax-type II-GN/GD). | SEQ ID NO: 200 |
| QANGADNQPGANGADDQPG (P. vivax-type II-DN/DD). | SEQ ID NO: 201 |
| CGCGAATTCAAGCGAACGGCGCCGATAATCAGCCGGCGGGTGCA | SEQ ID NO: 146 |
| (P. vivax-type IIB-ER1-wt-F). | |

3. Type-III (‘vivax-like’) CS-Repeat

The third P. vivax CS-epitope, which is quite different from the other two, is not associated with amino acid variation (see below) [Qari et al., Lancet, 1993. 341(8848): p. 780-783]. This sequence was cloned into the HBc expression system, and hybrids were produced that contained flanking dipeptide cloning site remnants.

| | |
|---|---|
| APGANQEGGAAAPGANQEGGAA (P. vivax-type III). | SEQ ID NO: 202 |

4. T cell Epitope at the C-Terminus of HBc

The insertion of the P. vivax Th epitope (Pv-UTC; YLDKVRATVGTEWTPCSVT; SEQ ID NO:196) into HBc and HBc hybrids was also performed using synthetic DNA fragments (Synthetic Genetics, San Diego Calif.). However, unlike B cell epitopes, which are inserted into the immunodominant loop region of the HBc gene, T cell epitopes are fused to the C-terminus of the HBc gene. Previously discussed cloning vectors were used for the insertion of both B and Th epitopes into HBc. The particle expressing just the Pv-UTC at the C-terminus has also been successfully made.

5. Combining B and T Cell Epitopes in a Single Particle

To combine B and Th epitopes into single HBc constructs, PCR is used to amplify N-terminal HBc fragments (AA 1-80, which contain the B cell epitopes), and C-terminal HBc fragments (AA 81-150, which contain the T cell epitopes). The fragments are ligated together and amplified again by PCR. Again, clones are verified by restriction endonuclease mapping and automated DNA sequence analysis (Lark Technologies, Houston Tex.). Details are essentially the same as for P. falciparum. Particles that contain each of the Type-I, -II and -III B cell epitopes and variants as well as the Pv-UTC, have been expressed and recovered.

### EXAMPLE 10

### Relative Immunogenicities of HBc Chimers

Relative immunogenicities of several HBc chimer immunogens were compared in mice using the IFA assay discussed previously. The results of those studies using two dose immunization regimens as before are shown below in Table 12.

TABLE 12

| Immunogen | IFA titer | Protection | Citation |
|---|---|---|---|
| P. berghei (CS-1) | 40,960 | 95% | A |
| P. yoelii (CS-3) | 12,800 | 95%* | B |
| P. falciparum (CS-2) | 1,200 | NT | A |
| P. falciparum | 5,200,000 | NT | — |
| (V12.Pf3.1) | | | |
| P. vivax (V2.PV-TIB) | 160,000 | NT | — |

[A = Schodel et al., J. Exp. Med., 1994, 180: 1037-1046.
B = Schodel at al., Behring Inst. Mitt., 1997 (98): p. 114-119.
NT = not tested.
*= protection for greater then 3 months.]

As is seen from the above data, titers of 10⁵-10⁶ for P. falciparum were achieved using a chimeric immunogen; this compares to titers of only 10⁴ for P. berghei and 10³ for P. falciparum using the replacement technology of Schodel et al.

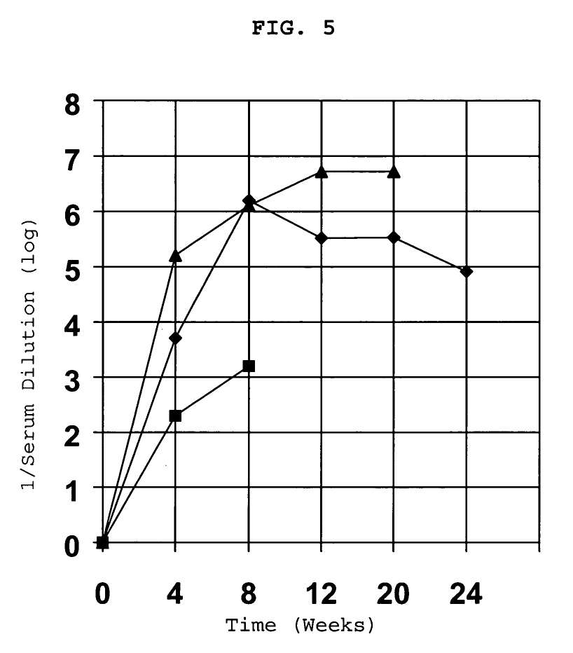

Mice were immunized with CS-2 or V12.Pf1 using 20 μg of particles on day zero and were boosted with 10 μg at four weeks. Mice immunized with particles from V12.Pf3 and V12.Pf3.1 were immunized using 20 μg of particles on day zero and were boosted with 10 μg at eight weeks using adjuvants as discussed before. Data showing the duration of the titers achieved are shown in FIG. 5, with data for use of V12.Pf3 particles being essentially identical to data with V12.Pf3.1 particles, and not shown.

### EXAMPLE 11

### Relative HBc Antigenicities

A series of studies was carried out to determine the relative antigenicities of several malarial HBc chimer particles toward two monoclonal antibodies (MoAb-3120 and MoAb-3105) as compared to native HBcAg (particle). These antibodies are specific to the loop region of HBc, and were the gracious gift of the Immunology Institute, Tokyo, Japan. Studies were carried out using the chimers of Table 5 that contain malarial epitopes inserted into HBc particles at various positions as antigens in ELISA assays with the monoclonals as probes. The results of these studies (as end point dilutions) are shown below in Table 13A, 13B, and 13C, and illustrate the substantial lack of antigenicity of a contemplated chimer toward monoclonal antibodies that bind to the loop region, the primary immunogen, of HBc. Put differently, monoclonal antibodies that bind specifically to the loop region of HBc barely recognize a contemplated chimer, if at all.

TABLE 13A

| | Anti-MoAb-3120 | Relative |
|---|---|---|
| Particle | End Point Dilution | Antigenicity |
| HBcAg | 625000 | 100 |
| V12.Pf3 | 80000 | 12.8 |
| V12.Pf3.1 | 20000 | 3.2 |
| V12.Pt3.2 | 10000 | 1.6 |
| V12.Pf3.3 | 10000 | 1.6 |
| V12.Pf3.4 | 80000 | 12.8 |
| V12.Pf3.5 | 40000 | 6.4 |
| V12.Pf3.6 | 80000 | 12.8 |
| V12.Pf3.8 | 80000 | 12.8 |
| V12.Pf3.9 | 160000 | 25.6 |
| V12.Pf3.10 | 10000 | 1.6 |
| V12.Pf3.11 | 80000 | 12.8 |
| V12.Pf3.12 | 80000 | 12.8 |

TABLE 13B

| | Anti-MoAb-3105 |
|---|---|
| Particle | End Point Dilution |
| HBcAg | 1,300,000 |
| V2.Pf1 | Zero |
| (78/79) | |
| V12.Pf1 | Zero |
| (78/79) | |
| V12.Pf3 | Zero |
| (78/79) | |
| V1.Pf1 | Zero |
| (77/78) | |
| V13.Pf1 | 1,300,000 |

An insertion into several sites in the immunodominant loop (including positions 77-78 or 78-79) totally eliminates binding of MoAb-3105. V13 is an insertion between residues 129 and 130, and is used as a control because the native HBc immunodominant loop remains intact in this construct.

TABLE 13C

| | Anti-MoAb-3120 |
|---|---|
| Particle | End Point Dilution |
| 77/78 V1.Pf1 | 102,400 |
| 78/79 V2.Pf1 | 400 |
| HBcAg | 409,600 |

These data show that insertion between residues 78 and 79 causes a more drastic reduction in anti-MoAb-3120 binding, as compared with insertion between residues 77 and 78.

### EXAMPLE 12

### Construction of a Modified Hepatitis B Core Protein Expression Vector

Using site-directed mutagenesis, a lysine codon (AAA) was introduced between amino acids E77 and P78 of the HBc gene, along a SacI (GAGCTC) restriction endonuclease site, to facilitate the genetic insertion of other codons for producing linker group-containing HBc particles. The insert thus had an amino acid residue sequence of KEL, where the EL is an artifact of the SacI site. The linker group-containing HBc protein was therefore 152 amino acid residues long. The construction of the pKK223-3-HBc152-K78 expression plasmid is described below.

Oligonucleotide primers P1F (SEQ ID NO:203) and P1R (SEQ ID NO:204, on the complementary strand) were used to amplify the 5′ end of the HBc gene (bases 1-234, amino acids 1-77), and simultaneously incorporate an NcoI restriction site (CCATGG) at the 5′ end, a SacI restriction site (GAGCTC) at the 3′ end of the amplified product, and a lysine codon (AAA) preceding the SacI site Oligonucleotide primers P2F (SEQ ID NO: 205) and P2R (SEQ ID NO: 206, on the complementary strand) were used to amplify the 3′ end of the HBc gene (bases 235-450, amino acids 78-149), and simultaneously incorporate a SacI restriction site (GAGCTC) at the 5′ end and a HindIII restriction site (AAGCTT) at the 3′ end of the amplified product.

The two PCR products (encoding amino acids 1-77 and amino acids 78-149) were cleaved with SacI, ligated together at their common SacI overhangs, cleaved with NcoI and HindIII and cloned into the expression plasmid pKK223-3 (Pharmacia), using standard techniques. The resulting plasmid was called pKK223-3-HBc152-K78.

This plasmid can be used for the expression of a HBc chimer bearing a lysine as a linker group in the immunodominant loop. The expressed HBc chimer spontaneously formed particles. The linker group-containing HBc of this Example thus had an insert corresponding to position 77 of the HBc of SEQ ID NO: 247, a chemically reactive lysine linker residue at a position corresponding to position 78 of the HBc of SEQ ID NO: 247, and was truncated at a position corresponding to position 149 of the HBc of SEQ ID NO:247.

A plasmid that encodes the above chimer and further includes a C-terminal cysteine residue can be prepared using the PCR techniques described in Example 1I, along with the preparation described immediately above. HBc chimer particles containing a C-terminal Cys residue and a linking residue that can be conjugated to an immunogenic hapten result from expression of the plasmid following the procedures described herein.

| | |
|---|---|
| Primer P1F | |
| TTGGGCCATGGACATCGACCCTTA | SEQ ID NO: 203 |
| Primer P1R | |
| GCGGAGCTCTTTTTCCAAATTAACACCCAC | SEQ ID NO: 204 |
| Primer P2F | |
| CGCGAGCTCGATCCAGCGTCTAGAGAGACC | SEQ ID NO: 205 |
| Primer P2R | |
| CGCAAGCTTAAACAACAGTAGTCTCCGGAAG | SEQ ID NO: 206 |

### EXAMPLE 13

### Modified Hepatitis B Core Particle Purification

Chimeric linker group-containing HBc particles of Example 12 were expressed in E. coli typically E. coli BLR or BL21 from Novagen (Madison, Wis.) or E. coli TB1 from Amersham (Arlington Heights, Ill.). The transfected E. coli [denoted HBc152-K78], expressed plasmid pKK223-3-HBc152-K78. The chimer linker group-containing HBc particles [HBc152(K78) particles] were purified via Sepharose® CL-4B—(Pharmacia) chromatography using established procedures.

In the nomenclature system used for these chimer molecules and particles, “HBc” denotes hepatitis B core protein sequence; “152” denotes the number of amino acid residues present in the chimer with lysine and two restriction site residues (glutamic acid and leucine; EL) being added to the HBc149 sequence from the SacI site; and “(K79)” denotes that the lysine (K) is added to the sequence after residue 78 as new residue 79. Chimer molecules and particles containing a cysteine residue as the C-terminal residue of the molecule, which are discussed hereinafter, are denoted as “+C”.

Because particles purify in a predictable manner, the monitoring of particle elution using simple spectroscopy (OD₂₈₀), in concert with SDS-PAGE analysis to assess purity of individual fractions prior to pooling, was sufficient to enable the routine purification of electrophoretically pure particles in high yield (5-120 mg/L cell culture). The spherical structure of the pure chimer linker group-containing HBc particles was clearly visible in an electron micrograph.

### EXAMPLE 14

### Chemical Coupling of Synthetic Peptides to Chimer Linker Group-Containing HBc Particles as Activated Carriers

The chimer linker group-containing HBc particle product of the expression plasmid pKK223-3-HBc152(K78) from Example 13 was assayed for its chemical reactivity compared with similarly expressed and purified “wild type” truncated hepatitis B core particle (HBc149), which is identical to HBc152(K78) except that it lacks the introduced lysine residue linker group and flanking five amino acids.

Synthetic peptides (haptens) were chemically conjugated to chimer linker group-containing HBc particles using succinimidyl 4-(N-maleimidomethyl)cyclohexane 1-carboxylate (SMCC), a water-soluble heterobifunctional cross-linking reagent used to form activated carriers. SMCC is reactive towards both sulfhydryl and primary amino groups, enabling the sequential conjugation of synthetic peptides to the activated carriers (HBc chimer particles whose primary amino groups have previously been modified with SMCC). Further, the 11.6 Ångstrom spacer arm afforded by SMCC helps to reduce steric hindrance between the hapten and the HBc carrier, thereby enabling higher coupling efficiencies.

Briefly, HBc152(K78) and HBc149 particles were separately reacted with a 5-fold excess of SMCC over total amino groups (native amino groups or native amino groups plus the one from the lysine residue of the insert) for 2 hours at room temperature in 50 mM sodium phosphate, pH 7.5, to form maleimide-activated HBc particles. Unreacted SMCC was removed by repeated dialysis against 50 mM sodium phosphate, pH 6.8. The SMCC derivitization of the HBc particles resulted in a minimal molecular weight increase that was not detectable by SDS-PAGE. However, the PAGE analysis did confirm the integrity of the HBc proteins prior to proceeding to the peptide conjugation step.

Synthetic peptides to be coupled to the chimer HBc particles as activated carriers were designed such that they had N-terminal cysteine residues to enable directional conjugation of peptide haptens to the primary amine on the side chain of the introduced lysine residue via the cysteine sulfhydryl of the hapten.

Table 14 shows the synthetic peptides derived from human cytochrome P450 enzymes that were chemically conjugated to HBc particle activated carriers to form HBc chimer particle conjugates containing pendently linked cytochrome P450 determinant haptens, or more simply, HBc chimer particle conjugates. The synthetic peptides were dissolved in 50 mM sodium phosphate, pH 6.8, to a concentration of 10 mg/ml. The synthetic peptides were then added, drop-wise, to a 5-fold excess over total amino groups in maleimide-activated, strategically modified HBc152(K78) particles, and permitted to react at room temperature for 2 hours. Maleimide-activated HBc149 particles were reacted with the two 2D6 peptides (2D6 and 2D6-C) as controls.

TABLE 14
Cytochrome P450 Haptens

| | | SEQ |
|---|---|---|
| Peptide Name | Sequence | ID NO |
| 1A1 (289-302) | CQEKQLDENANVQL | 207 |
| 1A2 (291-302) | CSKKGPRASGNLI | 208 |
| 2D6 (263-277) | CLLTEHRMTWDPAQPPRDLTE | 209 |
| 3A4 (253-273) | CVKRMKESRLEDTQKHRVDFLQ | 210 |
| 1A1-c | CMQLRS | 211 |
| 1A2-c | CRFSIN | 212 |
| 2D6-c | CAVPR | 213 |
| 2E1-c | CVIPRS | 214 |
| 2C-c | CFIPV | 215 |
| 3A3/4/7-c | CTVSGA | 216 |
| 3A5-c | CTLSGE | 217 |

### EXAMPLE 15

### Analysis of Chimer HBc Particle Conjugates

HBc chimer particle conjugates containing pendently linked to cytochrome P450 determinant haptens of Example 14 were analyzed by SDS-PAGE and immunoblots to determine if synthetic peptides had been successfully conjugated to HBc. The denaturing conditions of the electrophoresis procedure dissemble particles into their constituent subunits: HBc monomers. Because HBc monomers have a molecular weight of approximately 17,000 Da, it was simple to resolve HBc152(K78) particles chemically conjugated to either 1A1 (289-302), 1A2 (291-302), 2D6 (263-277) or 3A4 (253-273) peptides, as those peptides have a relative molecular mass of approximately 2,000 Da and therefore cause a visible increase in the molecular mass of the HBc protein monomers.

From the relative intensities of the conjugated and non-conjugated bands on SDS-PAGE, it was determined that approximately 50 percent of the HBc152(K78) monomers were covalently linked to hapten, whereas only about 5 percent of the “wild type” HBc149 particles were linked to hapten. The marked increase in the observed success in pendently linking hapten to the activated carrier supports the conclusion that the observed linking occurs via the inserted lysine as opposed to a lysine residue that is also present in the “wild type”.

The shift in mobility of HBc particles conjugated to shorter C-terminal P450-derived peptides (5- and 6-mers) is not as pronounced in the SDS-PAGE as that of the longer inhibitory peptides, but shifts of approximately 1 kDa were clearly evident in successfully coupled HBc152(K78) monomers. The chimeric HBc 152(K78) protein exhibited markedly enhanced ability to pendently link to a hapten over the “wild type” HBc149 particles, which showed minimal conjugation.

In the model of core particles propounded of icosahedral particles of either 180 or 240 associated core protein monomers [Conway et al. (1997) Nature, 386:91-94)], dimers of the relatively exposed immunodominant loop regions of the core monomers extend out from the assembled core particle into solution like spikes on a mace. The “spikes” are closely arranged spatially on the HBc particles. The strategic location of the introduced lysine residue on the tip of the spike minimizes the propensity for steric constraints to reactions linking haptens to assembled core particle.

A maximum of 50 percent of the strategically modified HBc monomers was successfully conjugated to the synthetic peptides of Cyt P450. That amount of pendent linkage corresponds to an average of one hapten attached per core protein dimer. This proposed distribution of hapten linkage to the strategically modified HBc particle is supported by PAGE results under semi-denaturing conditions that disassemble the particle while maintaining the dimer association.

HBc-2D6 particles prepared by peptide coupling were examined using immunoblots to confirm the presentation of the 2D6 polypeptide epitope. When probed with anti-HBc antisera, the chemically coupled particle yielded two different monomer bands representing monomers with and without the 2D6 polypeptide. Only the upper band of these blotted with anti-2D6 antisera, thereby confirming the correlation between mobility shift and attachment of the 2D6 polypeptide.

### EXAMPLE 16

### Strategic Lysine Insertions

To construct HBc particles with inserted lysine residues at every position in the immunodominant, surface-exposed loop region (amino acids 75-85), PCR was used to amplify the 5′ and 3′ fragments of the HBc gene and a single lysine codon was introduced via the oligonucleotide primers. The oligonucleotide primers and the resulting amino acid sequences are shown in SEQ ID NOs:220-241. The “wild type” sequences are SEQ ID NOs:218-219. These HBc chimers had a length of 150 residues with an added lysine at the postition noted by the number in each chimer and particle name.

In order to prepare lysine inserts at positions 75 to 84 [HBc150(K75) through HBc150(K84)], the pairs of PCR fragments were digested with the restriction endonuclease MseI, which recognizes the sequence, TTAA. The modified gene was restored by ligating the oligonucleotide primer (containing the lysine) at the convenient MseI restriction site located at nucleotides 221-224. For HBc-K85 (SEQ ID NOs:240-241) it was necessary to prepare two fragments that were ligated at a common XhoI restriction site (CTCGAG) that is not present in the wild type gene, but could be introduced at position 239-244 without altering any amino acids.

TABLE 15

Lysine Insertion Mutants of HBc
in the Immunodominant Loop

| | | |
|---|---|---|
| Name | Sequence | SEQ ID NO: |

| | | |
|---|---|---|
| Wild Type HBc | TWVGVNLEDPASRDLLVVSYV | 218 |
| HBc150K75 | TWVGVKNLEDPASRDLVVSYV | 220 |
| HBc150K76 | TWVGVNKLEDPASRDLVVSYV | 222 |
| HBc150K77 | TWVGVNLKEDPASRDLVVSYV | 224 |
| HBc150K78 | TWVGVNLEKDPASRDLVVSYV | 226 |
| HBc150K79 | TWVGVNLEDKPASRDLVVSYV | 228 |
| HBc150K80 | TWVGVNLEDPKASRDLVVSYV | 230 |
| HBc150K81 | TWVGVNLEDPAKSRDLVVSYV | 232 |
| HBc150K82 | TWVGVNLEDPASKRDLVVSYV | 234 |
| HBc150K83 | TWVGVNLEDPASRKDLVVSYV | 236 |
| HBc150K84 | TWVGVNLEDPASRDKLVVSYV | 238 |
| HBc150K85 | TWVGVNLEDPASRDLKVVSYV | 240 |

To purify the linker group-containing HBc chimers, cleared cell lysates from a 1L fermentation were precipitated with 45% ammonium sulfate and the resultant pellet subjected to gel filtration using Sepharose® (Pharmacia)CL-4B chromatography (2.5 cm×100 cm). Particulate HBc has a characteristic elution position when analyzed using this type of column, independent of the amino acid insertions made to the particle. The eleven linker group-containing HBc chimer particles prepared for this study were analyzed using this procedure and the elution profiles were measured spectrophotometrically at an absorbance of 280 nm.

Three of the linker group-containing HBc chimer particles prepared from constructs [HBc150(K75), HBc150(K77), and HBc150(K79)] were produced at levels of between 50 and 100 mg/L, which is comparable with typical yields for wild-type, unmodified HBc particles, e.g. HBc149 particles. Linker group-containing HBc chimer particles of four of the constructs [HBc150(K76), HBc150(K78), HBc150(K81), and HBc150(K82)] were produced at relatively low levels (between 1 and 20 mg/L). Finally, four of the particles [HBc150(K80), HBc150(K83), HBc150(K84), and HBc150(K85)] were produced at levels deemed to be barely detectable (less than 1 mg/L). The yields of these expression products are shown in Table 16, below.

TABLE 16
Purified Lysine-Containing Chimer
HBc Particles from a One L Fermentation

| Particle | Yield (mg/L) |
|---|---|
| HBc150 (K75) | 77 |
| HBc150 (K76) | 5 |
| HBc150 (K77) | 74 |
| HBc150 (K78) | 10 |
| HBc150 (K79) | 94 |
| HBc150 (K80) | 0 |
| HBc150 (K81) | 17 |
| HBc150 (K82) | 1 |
| HBc150 (K83) | 0 |
| HBc150 (K84) | 0 |
| HBc150 (K85) | 0 |

As before, a plasmid that encodes the above chimer and further includes a C-terminal cysteine residue can be prepared using the PCR techniques described above or in Example 1I by insertion of a Cys codon just upstream from the termination codon, along with the preparation described immediately above.

### EXAMPLE 17

### Chimers with HIV Sequences

Recombinant chimer particles were prepared in which the HIV-1 gp41 sequence of positions 631-665 was present between HBc residues 78 and 79. One preparation contained a C-terminal Cys residue (SEQ ID NOs: 272 and 273), whereas the other did not and was terminated at the valine of HBc position 149 (SEQ ID NOs: 270 and 271). The particles with no terminal Cys were expressed using the V2 vector discussed in Example 1B, whereas the Cys-terminated particles were expressed from a vector prepared as discussed in Example 1I. Those constructs are referred to as V2.HIV11.1 and V16.HIV11.1, respectively. The yields on expression were 1.6 mg/L and 12.4 mg/L, respectively, thereby illustrating an almost 8-fold increase in yield for the particles assembled from the Cys-terminated protein.

The sequence of the HIV B cell epitope is shown below, as are the coding and complementary DNA sequences for that epitope. The HIV sequence conveniently ends with a C-terminal EL residue and begins with added N-terminal GI residues, so that there are two added (heterologous) residues in total that are neither from the HBc sequence nor from the inserted peptide sequence.

| | |
|---|---|
| Inserted B cell epitope sequence | |
| GIQWMEWDREINNYTSLIHSLIEESQNQQEKNEQEL | SEQ ID NO: 242 |
| Coding sequence | |
| 5′ AATTTGGATGTGGGAAGATCGTGAGATCAACAATTATACCAGCCTGATACATT | SEQ ID NO: 243 |
| CTTTAATTGAAGAGTCCCAGAACCAACAGGAGAAAAATGAACAAGAGCT | |
| Complementary sequence | |
| 5′ CTTGTTCATTTTTCTCCTGTTGGTTCTGGGACTCTTCAATTAAAGAATGTATC | SEQ ID NO: 244 |
| AGGCTGGTATAATTGTTGATCTCACGATCTTCCCACATCCA | |

### EXAMPLE 18

### Comparative Expression

A similar comparative expression study was carried out using the previously described HBc150(K77) vector that expresses a chimer molecule containing a lysine between residues 76 and 77 of HBc (along with two exogenous residues on either side of the added lysine) and a similar vector that also contained a Cys residue at the C-terminus of the protein. The latter vector was prepared by the techniques discussed before by using a C-terminal PCR primer that contained a codon for Cys between the Val-149 and stop codons. In a paired expression study, the former vector expressed particles in an amount of 55 mg/L, whereas the latter vector expressed particles in an amount of 60 mg/L.

### EXAMPLE 19

### Preparation of C-Terminus Truncated HBc Chimer Genes and Particles

The HBc gene was amplified using HBc-NcoI-fwd (shown hereinafter) in concert with each of the following reverse primers: HBc138+139C-H3-rev, HBc139-H3-rev, and HBc140-H3-rev (shown hereinafter) to generate the following HBc genes: HBc140, HBc139 and HBc138+139C. The PCR products were cut with NcoI and HindIII and cloned into pKK223-3N, which was prepared by cutting with same two enzymes. Plasmids were then transformed into E. coli strain TB1 and grown for 24 hours in 500 mL of TB media supplemented with 8 ml g/L glucose and 50 g/mL ampicillin. Particle production was determined by analyzing crude E. coli preparations using a Sepharose® CL-4B sizing column (Pharmacia), whereby particles are associated with a characteristic elution position.

Thus, five grams of harvested cells were lysed in 25 mL of 50 mM Tris-HCl buffer, pH 8.0, 10 mM EDTA using a French press. The lysate was clarified by centrifugation at 16,000 rpm (JA-30.50 Ti rotor, Beckman) for 20 minutes. Ammonium sulfate precipitation (45%) was used to precipitate particles, and the precipitate was recovered by centrifugation at 16,000 rpm (JA-30.50 Ti rotor, Beckman) for 20 minutes. The pellet so formed was resuspended in 5 mL of 50 mM Tris-HCl, pH 8.0, 10 mm EDTA and dialyzed against the 20 mm Tris-HCl, pH 8.0 until soluble. The material was then loaded onto a Sepharose CL-4B chromatography column (2.5×100 cm) and allowed to run at a flow rate of 1 mL/minute for 500 minutes, by which time all material was eluted. Elution of particles was monitored at 280 nm.

Based upon the elution profiles, HBc 140 makes particles, whereas HBc 139 does not. Particles also were not formed by the addition of a cysteine at position 139 of a particle that otherwise ended at residue 138. Vectors were constructed using DNA of SEQ ID Nos: 275, 146, 159, 160, 155, 156, 153 and 154 shown previously.

### EXAMPLE 20

### Preparation of Vector for Preparation of HBc Particles for Use in Humans

A. Preparation of Vector V17Pf3.1

To manufacture the particle V12.Pf3.1 (SEQ ID NOs: 268 and 269) in a manner suitable for human administration, it was necessary to express the particle using an expression system that did not require the use of ampicillin to ensure plasmid maintenance. To achieve this, the gene coding for the particle, along with the necessary upstream regulatory sequences, was inserted into a new plasmid that utilizes kanamycin as the selectable marker. The new plasmid (V17.Pf3.1) was synthesized using a two step cloning procedure:

Step 1: The plasmid pKK223-3N-V12 was digested with the restriction enzymes BamHI and HindIII to yield two DNA fragments of 801 and 4869 bp. In addition, the commercially available plasmid pREP4 (Qiagen) was cut with BglII and HindIII to yield two fragments of 320 bp and 3420 bp. The 3420 bp and 801 bp fragments were ligated to create plasmid V17. (It is noted that BglII and BamHI digested DNAs can be ligated by virtue of their common ‘overhang’ sequences, although neither BglII or BamHI can cut the resultant fragment). The V17 plasmid, therefore, contains the HBc149 gene, complete with Pf-UTC sequence fused to the C-terminus, and EcoRI and SacI restriction sites in the immunodominant loop region to enable insertion of epitopes between D78 and P79 of the HBc gene.

Step 2: The second step was to insert the Pf3.1 version of the Pf CS-repeat epitope into the immunodominant loop region of the gene. This was achieved by digesting V17 with SacI and EcoRI to yield 15 bp and 4206 bp DNA fragments. Annealed oligonucleotides encoding the Pf3.1 epitope were ligated with the 4206 bp fragment to yield V17.Pf3.1, a 4275 base pair plasmid. In addition to the gene that encodes the 195 amino acid malaria vaccine candidate, this plasmid contains a gene for the lac repressor (lac I) to force any gene under lac promoter control to be fully repressed until induced by isopropylthiogalactoside (IPTG). It also has a kanamycin resistance gene to permit positive selection via the addition of kanamycin to culture media. The plasmid has the replication origin of pACYC 184 and is not considered to be a high copy number plasmid.

| | | | Amino | Molecular |
|---|---|---|---|---|
| Gene | Start | Stop | Acids | Weight (kDa) |
| Lac I | 2128 | 3087 | 319 | 34.1 |
| V17.Pf3.1 | 281 | 868 | 195 | 21.7 |
| KmR | 4259 | 3465 | 264 | 29.1 |

A suitable host for V17.Pf3.1 is E. coli BLR, a rec A derivative of E. coli BL21, and a common strain used for the production of recombinant proteins (available for purchase from Novagen). E. coli BLR was selected as a host organism for expression because of its increased genetic stability, as well as its ability to produce assembled particles in soluble form (not in inclusion bodies).

B. Expression of Particles Using Plasmid V17.Pf3.1

E. coli (Strain BLR) containing the V17.Pf3.1 plasmid were streaked onto an LB agar plate supplemented with 25 μg/mL kanamycin and 10 μg/mL tetracycline, then incubated at 37° C. for 16-20 hours. A single colony was then used to inoculate 3 mL of TB-Phy medium in a sterile culture tube, supplemented with 25 μg/mL kanamycin. The tube was incubated overnight (about 18 hours) on a shaker at 37° C. and about 200 rpm.

The following morning, 100 mL of TB-Phy medium was warmed to 37° C. One mL of the overnight culture was removed and used to inoculate the flask, which was then incubated on a shaker at 37° C. at about 200 rpm for six hours.

The fermentor (Biostat™ UE20) was inoculated with 100 mL of inoculum with the fermentor conditions set as follows:

| | |
|---|---|
| Agitation | 400 rpm |
| Temperature | 37° C. |
| Aeration | air, 10 liters per minute |
| pH | 7.0, uncontrolled |

The A₆₀₀ value was measured for the first sample, and for samples every 20-30 minutes thereafter to monitor A₆₀₀. An IPTG solution was prepared by dissolving 62 mg IPTG in 10-15 mL water. When the A₆₀₀ value reached 0.5, the filter-sterilized IPTG solution was aseptically added to the fermentor through a syringe. The incubation was continued until next day (e.g. about another 10-24 hours).

At 14 hours after induction, the fermentor temperature was set to 15° C. Harvesting of cells was started by centrifugation in a Beckman® J2-MC centrifuge with following conditions:

| | |
|---|---|
| Rotor | JA10 |
| Speed | 7,500 rpm |
| Temperature | 4° C. |
| Time | 9 minutes |

The cells were harvested by freezing into liquid nitrogen.

C. Purification of Particles Expressed by Vector V17.Pf3.1/BLR

The biomass of harvested cells was resuspended in 50 mM sodium phosphate, pH 6.8, and lysed using a French Pressure cell at 16,000 psi. The cell debris was removed by centrifugation using a Beckman® J2-MC centrifuge and the following conditions.

| | |
|---|---|
| Rotor: | JA20 |
| Speed: | 15,000 rpm |
| Temperature: | 4° C. |
| Time: | 30 minutes. |

The volume of the resultant supernatant was measured and 277 g/L of solid ammonium sulfate were slowly added to the supernatant. The mixture was stirred at 4° C. for 30 minutes. The solution was centrifuged in Beckman® J2-MC centrifuge with the following conditions.

| | |
|---|---|
| Rotor: | JA20 |
| Speed: | 15,000 rpm |
| Temperature: | 4° C. |
| Time: | 30 minutes |

The precipitate was then resuspended in a minimal volume of 50 mM sodium phosphate buffer and then dialyzed against the same buffer for one hour with stirring. The dialyzed solution was centrifuged in Beckman® J2-MC centrifuge with the following conditions.

| | |
|---|---|
| Rotor: | JA20 |
| Speed: | 15,000 rpm |
| Temperature: | 4° C. |
| Time: | 15 minutes |

The supernatant was recovered and then subjected to gel filtration chromatography.

System: Pharmacia Biotech AKTA™ Explorer

Buffer B (elution solvent): 50 mM Sodium phosphate buffer (pH 6.8).

Column: Millipore Vantage™ VL44×1000 column (44 mm diameter, 1000 mm height, Catalog No.: 96441000)

Resin: 1.5 liter Sepharose® CL-4B manufactured by Pharmacia

Detector: UV at 210, 254 and 280 nm.

Fraction: 15 mL

The column was eluted with buffer B at 2 mL per minute. Particle-containing fractions were identified using SDS-PAGE and pooled. The salt concentration of the pooled material was adjusted to 5M by adding sodium chloride.

Hydrophobic Interaction Chromatography:

System: Pharmacia® Biotech AKTA™ Explorer (System No.: 18111241 001152, University of Iowa ID No.: 540833.)

Buffer A: 50 mM sodium phosphate buffer (pH 6.8)+5 M NaCl. (The buffer was degassed for 30 minutes daily, before use.)

Buffer B (elution solvent): 50 mM sodium phosphate buffer (pH 6.8). (The buffer was degassed for 30 minutes daily, before use.)

Hydrophobic Interaction Chromatography Using ToyoPearl® Ether 650 Resin

Column: Millipore Vantage™ VL44×250 column (44 mm diameter, 250 mm height, Catalog No.: 96440250)

Resin: 200 mL Toyopearl® ether 650 HIC resin, manufactured by Tosohaas

Detector: UV at 210, 254, and 280 nm

Fraction: 15 mL

The column was equilibrated with 5 column volumes (CV) of buffer A for a one hour time prior to starting purification, using a flow rate of 20 mL/minute. The retentate containing 5 M salt was then loaded at a rate of 20 mL/minute. The column was washed with 2 CV of buffer A, washed with 2 CV of 10% buffer B, eluted with 3 CV of 40% buffer B, and (finally eluted) with 100% buffer B. Fractions were completely analyzed for proteins of interest by SDS PAGE analysis. Pure fractions were combined together, and a protein estimation using a Bradford assay was carried out.

Hydrophobic Interaction Chromatography Using Butyl Resin

Column: Millipore Vantage™ VL44×250 column (44 mm diameter, 250 mm height, Catalog No.: 96440250)

Resin: 200 mL Toyopearl® Butyl 650-S HIC resin, manufactured by Tosohaas

Detector: UV at 210, 254 and 280 nm

Fraction: 15 mL

The column was equilibrated with 5 column volumes (CV) of 40% buffer B for one hour prior to starting purification, using a flow rate of 20 ml/min. The combined fractions from ether HIC were loaded at a rate of 20 mL/minute. The column was washed with 2 CV of 40% buffer B, washed with 2 CV 90% B, and eluted with 4 CV of WFI.

Fractions were analyzed for protein of interest by SDS PAGE analysis. Pure fractions were combined together

Hydroxyapatite Column Chromatography

Column: Millipore Vantage™ VL16×250 column (16 mm diameter, 250 mm height, Catalog No.: 96160250)

Resin: 20 ml Ceramic Hydroxyapatite (Catalog No. 158-2200)

Detector: UV at 215, 254 and 280 nm

Fraction: 15 mL

The column was equilibrated with 5 column volumes (CV) of 20 mM sodium phosphate buffer, flow rate: 5 mL/min. Load combined fractions eluted from butyl HIC at 5 mL/min. Wash the column with 20 mM sodium phosphate buffer until A280 drops to baseline. Fractions were analyzed for protein of interest by SDS PAGE analysis. Pure fractions were combined together.

Desalting

Column: Prepacked desalting column, HiPrep™ 26/10, Pharmacia

Resin: 20 mL Ceramic Hydroxyapatite (Catalog No. 158-2200)

Detector: UV at 215, 254 and 280 nm

Fraction: 15 mL

The column was equilibrated with 5 CV of 15 mM Acetate Buffer, pH 6.0. The pooled fractions from the hydroxyapatite column were loaded onto the column, and then eluted with 15 mM Acetate Buffer, pH 6.0, at a flow rate of 20 mL/min. Fractions were analyzed for protein of interest by SDS PAGE analysis. Pure fractions were combined together, and protein estimation was carried out using a Bradford assay. The pure fraction was assayed for endotoxin level, and finally passed through a 0.22-micron filter for terminal filtration.

### EXAMPLE 21

### Comparative Expression of Chimers with Cytochrome P450 Sequences

Recombinant chimer particles were prepared in which the human cytochrome P450 1A1 sequence of positions 290-302 was present between HBc residues 78 and 79. One preparation contained a C-terminal Cys residue, whereas the other did not and was terminated at the valine of HBc position 149. The particles with no terminal Cys were expressed using the V2 vector discussed in Example 1B, whereas the Cys-terminated particles were expressed from a vector prepared as discussed in Example 1I. Those vectors are referred to as V2.1A1(290-302) and V16.1A1(290-302), respectively. The yields on expression were 2.7 mg/g cells, 36 mg/L culture and 8.8 mg/g, 144 mg/L, respectively, thereby illustrating the ability of the terminal cysteine modification to stabilize chimer molecule particle production and yield.

The sequence of the P450 1A1 peptide is shown below, as are the coding and complementary DNA sequences for that epitope. The P450 1A1 sequence begins with a N-terminal GI and ends with a C-terminal EL residue sequence, so that there are only four added (heterologous) residues, in total, that are neither from the HBc sequence, nor that of the inserted peptide sequence.

| | |
|---|---|
| Inserted B cell epitope sequence | |
| (GI)QEKQLDENANVQL(EL) | SEQ ID NO: 280 |
| Coding sequence | |
| 5′ CAAGAAAAACAGCTAGACGAAAACGCAAATGTACAGCTC | SEQ ID NO: 74 |
| Complementary sequence | |
| 5′ CGAGCTGTACATTTGCGTTTTCGTCTAGCTGTTTTTCTTG | SEQ ID NO: 71 |

### EXAMPLE 22

### Preparation of Vectors to Express

Particles with a Cysteine Residue Prior to C-Terminal Fused Epitope

To prepare particles with a single cysteine after V149 of the HBc gene, followed by a T cell epitope, a PCR primer was synthesized (SEQ ID NO: 282). This primer, in conjunction with HBc149/NcoI-F (SEQ ID No: 67), was used to amplify the HBc gene to produce a version of HBc having a single cysteine codon introduced directly after V149, as well as EcoRI and HindIII restriction sites (after the introduced cysteine). The 478 bp PCR product was cut with NcoI and HindIII and cloned into pKK223-3N.

SEQ ID No. 281

```
                                                  C V V T T E P
5′ GCAAGCTTACTATTGAATTCCGCAAACAACAGTAGTCTCCGG
          HindIII EcoRI
```

SEQ ID No: 282

The resultant plasmid was then cut with EcoRI and HindIII and the annealed oligonucleotides coding for the Pf/CS-UTC (PF/CS326-345; SEQ ID Nos: 121 and 122) ligated into the plasmid. This plasmid was then used as the template in a PCR reaction along with the primers HBc-P79/SacI-F (SEQ ID No: 73) and Pf/CS (C17A) (SEQ ID No: 145) the resultant PCR product (307 bp) coded for amino acid residues 79 through 149 of HBc, followed by the introduced cysteine, followed by the Pf/CS-UTC sequence having the C17A mutation, and flanked by SacI (5′) and HindIII (3′) restriction sites. This fragment was cut with SacI and HindIII and ligated with the plasmid V2.Pf1 [encoding the malarial (NANP)₄ epitope] that had been cut with the same two enzymes.

The resultant gene codes for a 190 amino acid residue HBc chimera having (NANP)₄ inserted between amino acids 78 and 79 of HBc, (flanked by the Gly-Ile and Glu-Leu sequences derived from the EcoRI and SacI restriction sites respectively) and the C17A version of the Pf/CS326-345 at the C terminus. The single cysteine was therefore located between V149 of HBc and the Gly-Ile linker sequence (derived from the EcoRI restriction site) located prior to the first amino acids of the Pf/CS326-345(C17A) [Pf/CS-UTC(C17A)] T cell epitope (see SEQ ID No. 284).

This hybrid particle was expressed, purified and analyzed for stability by incubating at 37° C. for several weeks. The stability of this particle (V12.Pf1(C17A)C150) was compared to V12.Pf1, with the only difference between the two particles being the position of the cysteine residue. For V12.Pf1 the cysteine is followed by three amino acid residues (SVT) at the C-terminus of the protein (SEQ ID No: 283), whereas for V12.Pf1(C17A)C150 the cysteine is followed by 22 additional amino acid residues (SEQ ID No: 284).

| | |
|---|---|
| V12.Pf1 | |
| TTVV GI EYLNKIQNSLSTEWSPCSVT | SEQ ID No: 283 |
| V12.Pf1(C17A)C150 | |
| TTVV C GI EYLNKIQNSLSTEWSPASVT | SEQ ID No: 284 |

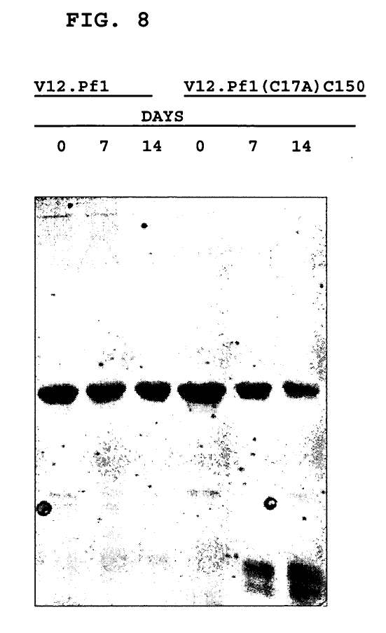

The effect of inserting the cysteine residue between HBc and the T cell epitope (V12.Pf1(C17A)C150) was to create a particle that was significantly more stable than a similar particle without the C terminal cysteine (V12.Pf1(C17A)). This was evident from the fact that unlike V12.Pf1(C17A), V12.Pf1(C17A)C150 could be easily purified without a significant degree of degradation of monomers (compare T=O for these particles in FIGS. 4 and 8); further, V12.Pf1(C17A)C150 was significantly more stable than V12.Pf1(C17A) following incubation at 37° C. After 14 days at 37° C., V12.Pf1(C17A) monomers are totally degraded (FIG. 4), whereas V12.Pf1(C17A)C150 monomers are only partially degraded (FIG. 8).

It was apparent that V12.Pf1(C17A)C150 was not as stable V12.Pf1 (FIG. 8). These data indicate that the stabilizing effects of a single C-terminal cysteine residue are most effective when placed at or near, e.g., within five residues of, the C-terminus of the HBc chimer.

### EXAMPLE 23

### Analytical Gel Filtration Analysis of Hybrid Particles

Analytical gel filtration analysis of purified hybrid HBc particles was performed using a 25 mL Superose® 6 HR 10/30 chromatographic column (Amersham Pharmacia # 17-0537-01) and a BioCAD™ SPRINT Perfusion Chromatography System. The UV detector was set to monitor both wavelengths of 260 and 280 nm. The column was equilibrated with 3 column volumes (CV; about 75 mL) of buffer (50 mM NaPO₄, pH 6.8) at a flow rate of 0.75 mL/minute.

The particles to be analyzed were diluted to a concentration of 1 mg/mL using 50 mM NaPO₄, pH 6.8. 200 Microliters (μL) of the sample were then loaded onto a 200 μL loop and injected onto the column. The sample was eluted from the column with 50 mM NaPO₄, pH 6.8 at a flow rate of 0.75 mL/minute.

Several particles containing C-terminal cysteine residues or similar particles free of such cysteines were analyzed using the above procedure. Integration of the 280 nm trace was carried out using BioCAD™ software (PerSeptive™) to provide the results in Table 17, below.

TABLE 17

Percent After
Purification

| | | Non |
|---|---|---|
| Particle | Particulate | Particulate |
| V2.1A1 (290 to 302) | 43 | 57 |
| V16.1A1 | 96 | 4 |
| (290 to 302)* | | |
| V12.Pf1 (C17A) | 67 | 33 |
| V12.Pf1 | 100 | 0 |
| (C17A) + C150* | | |
| V12.Pf1* | 98 | 2 |
| HBc150 (K77) | 40.1 | 59.9 |
| HBc150 (K77) + C* | 100 | 0 |
| HBc150 (K79) | 59 | 41 |
| HBc150 (K79) + C* | 100 | 0 |
| V2.Pf1 + CF/HBc74-87 + C* | 97.8 | 2.2 |
| V2.Pf1 + CF/HBc74-87 | 80.7 | 19.3 |

*C-terminal cysteine-stabilized particles.

Purified particles were assayed for the percentage of particles and then incubated in aqueous solution at 37° C. as discussed before. The compositions were assayed for stability after fourteen days of incubation. The results of this analysis are shown in Table 18, below.

TABLE 18

Percent Particles
Following
Incubations
at 37° C. (Days)

| Particle | Zero | 14 |
|---|---|---|
| V12.Pf1* | 98 | 96 |
| V12.Pf1 (C17A) | 67 | 63 |
| V12.Pf1 (C17A) + C150* | 100 | 98 |

*See the note to Table 17.

FIG. 8 shows the results of a SDS-PAGE analysis of the particles of Table 18 at days zero, 7 and 14 following incubation at 37° C. Results of a densitometric analysis of that a SDS-PAGE analysis are shown in Table 19, below.

TABLE 19

| | Percent Full Length Monomer | | |
|---|---|---|---|
| | Following Incubation at 37° C. Days | | |
| Particle | Zero | 7 | 14 |
| V12.Pf1* | 100 | 94 | 93 |
| V12.Pf1 (C17A) | 100 | 13 | 1 |
| V12.Pf1 (C17A) + C150* | 100 | 83 | 63 |

*See the note to Table 17.

The particles of Tables 18 and 19 and control particles of Example 16 with and without a C-terminal Cys residue were analyzed for immunogenicity in BALB/c mice via intraperitoneal injection using 20 μg of the respective particles in phosphate buffered saline (pH 7.4) in the absence of adjuvant, contrary to the results reported in Example 4. Sera were analyzed two weeks after immunization using an ELISA with HBc particles (Anti-HBc) or (NANP)₅ synthetic peptide [Anti-(NANP)ₙ] as the solid phase capture antigen. The results of this study are shown in Table 20, below

TABLE 20

End Point Titer

| Particle | Anti-HBc | Anti-(NANP)ₙ |
|---|---|---|
| V12.Pf1 (C17A) | 10,240 | 0 |
| V12.Pf1 | 10,240 | 2,560 |
| (C17A) + C150* | | |
| V12.Pf1* | 10,240 | 10,240 |
| HBc150 (K77) | 40,960 | 0 |
| HBc150 (K77) + C* | 163,840 | 0 |

*See the note to Table 17.

The data from this study are interpreted to mean that the C-terminal cysteine-stabilized particles are more stable immediately on production as well as after incubation at 37° C. for various time periods. The stabilized particles also exhibit enhanced immunogenicity even in the absence of adjuvant. In addition, although particulate matter is present in the non-stabilized material such as V12.Pf1(C17A), there are no monomeric chimeric proteins after fourteen days of incubation and the material present does not induce antibodies toward the initially introduced heterologous B cell epitope sequence, here a malarial immunogen.

### EXAMPLE 24

### Chimers Containing Beta-Amyloid Protein Epitope Sequences

Antibodies to the 42 amino acid beta-amyloid precursor protein fragment have been proposed as a therapeutic and prophylactic vaccine for treating Alzheimer's Disease (REF) [Schenk et al. (Jul. 8, 1999) Nature, 400(6740):116-117]. The C-terminus of that fragment contains a region that is extremely hydrophobic, and therefore potentially problematic for expression at the surface of chimeric HBc particles.

Therefore, in addition to a particle containing the complete 42 amino acid sequence [V16.β-Am(1-42)], three other particles were constructed that contain only the relatively hydrophilic regions: amino acid residues 1-17 [V16.β-Am(1-17)], amino acid residues 22-32 [V16. β-Am(22-32)], and amino acid residues 1-32 [V16.β-Am(1-32)]. Chimeric genes coding particles V16.β-Am(1-17) and V16.β-Am(22-32) were constructed by annealing complimentary oligonucleotides and inserting them into the plasmid V16 that had previously been digested with EcoRI and SacI.

| | |
|---|---|
| β-Am(1-17)-T | |
| 5′-AATTGATGCGGAATTTCGTCATGACAGCGGCTATGAGGTGCACCATC- | SEQ ID NO: 296 |
| AGAAACTGGAGCT | |
| β-Am(1-17)-B | |
| 5′-CCAGTTTCTGATGGTGCACCTCATAGCCGCTGTCATGACG- | SEQ ID NO: 297 |
| AAATTCCGCATC | |
| β-Am(22-32)-T | |
| 5′-AATTGAAGATGTCGGTTCTAACAAGGGGGCAATTATCGAGCT | SEQ ID NO: 298 |
| β-Am(22-32)-B | |
| 5′-CGATAATTGCCCCCTTGTTAGAACCGACATCTTC | SEQ ID NO: 299 |

For chimeric genes containing residues 1-42 [V16. β-Am(1-42)] and 1-32 [V16. β-Am(1-32)], the oligonucleotides β-Am(1-32/42)-T and β-Am(1-42)-B or β-Am(1-32)-B were annealed, and then filled-in to make the fragment completely double stranded using 5 cycles of melting (94° C.) and filling-in (72° C.). The reactions were performed in a total volume of 100 μL using Vent polymerase (NEB), dNTPs (250 μM) and the annealed fragments (250 nM). Two microliters of these reaction products were then used as templates in two PCR reactions to prepare the fragments coding for residues 1-32 and 1-42, flanked by EcoRI and SacI restriction sites. (Note: Leu codon (CTG) is introduced by the primer “β-Am(L+1-32/42)-5′—PCR” and precedes the first β-Am amino acid in the following two constructs to restore EcoRI site for the cloning purposes).

Oligonucleotides for Preparation of β-Amyloid Residue 1-32 and 1-42 Fragments:

| | |
|---|---|
| β-Am(1-32/42)-T | |
| 5′-GCGGGAATTGATGCGGAATTTCGTCATGACAGCGGCTATGAGGTG- | SEQ ID NO: 300 |
| CACCATCAGAAACTGGTTTTCTTTGCCGAAGATGTCG | |
| β-Am(1-42)-B | |
| 5′-GCGGAGCTCCGCTATGACAACCCCACCCACCATTAAGCCGAT- | SEQ ID NO: 301 |
| AATTGCCCCCTTGTTAGAACCGACATCTTCGGCAAAGAAAA | |
| β-Am(1-32)-B | |
| 5′-GCGGAGCTCGATAATTGCCCCCTTGTTAGAACCGACAT- | SEQ ID NO: 302 |
| CTTCGGCAAAGAAAA | |
| PCR Primers for residue 1-42 amplification | |
| β-Am(L+1-32/42)-5′-PCR | |
| 5′-GCGGGAATTCTGGATGCGGAATTTCGTCATG | SEQ ID NO: 303 |
| β-Am(1-42)-3′PCR | |
| 5′-GCGGAGCTCCGCTATGA | SEQ ID NO: 304 |
| PCR Primers for residue 1-32 amplification | |
| β-Am(L+1-32/42)-5′-PCR | |
| 5′-GCGGGAATTCTGGATGCGGAATTTCGTCATG | SEQ ID NO: 305 |
| β-Am(1-32)-3′PCR | |
| 5′-GCGGAGCTCGATAATTGC | SEQ ID NO: 306 |

### EXAMPLE 25

### Influenza M2 Constructs

Recently, Neirynck et al., (October 1999) Nature Med., 5(10):1157-1163 and Wo 99/07839 reported the fusion of the 24 amino acid extracellular domain of M2 to the N-terminus of full-length HBc particles (HBc183), lacking amino acid residues 1-4. A schematic representation of that construct referred to herein as IM2HBc is shown below in which the 24-mer is linked to the N-terminus of HBc.

| | |
|---|---|
| IM2HBc | |
| MSLLTEVETPIRNEWGCRCNDSSD-HBc(5-183) | SEQ ID NO: 307 |

In one illustrative preparation, the M2 epitope was inserted into the immunodominant loop of hepatitis B core and particles referred to as ICC-1475 were successfully expressed and purified using techniques discussed previously for such insertions and purifications. A mutated version of the M2 epitope, in which two cysteine residues at M2 native positions 17 and 19 were substituted by alanine residues, was also expressed in the immunodominant loop (ICC-1473) and the resulating particles purified. These two particles are illustrated schematically below.

| | |
|---|---|
| ICC-1475 | |
| HBc(1-78)-GI-SLLTEVETPIRNEWGCRCNDSSD-EL-HBc(79-149) | SEQ ID NO: 308 |
| ICC-1473 | |
| HBc(1-78)-GI-SLLTEVETPIRNEWGARANDSSD-EL-HBc(79-149)-C | SEQ ID NO: 309 |

The ICC-1473 construct yielded approximately 7-fold more purified particles when compared with the native sequence (ICC-1475). It remains to be determined if the mutation of the cysteine residues alters protective potential of the particles. However, epitopes delivered on the immunodominant loops of HBc are usually significantly more immunogenic as compared to when they are fused to other regions (including the N-terminus), and resulting particles exhibit reduced anti-HBc immunogenicity.

Particles have also been prepared in which the M2 N-terminal 24-mer epitope was fused to the N-terminus of C-terminal truncated hepatitis B core particles. That construct (ICC-1438) also contained the N-terminal pre-core sequence (SEQ ID NO:310). A similar construct was prepared that contained a single cysteine residue at the end of the hybrid protein (ICC-1492), in this case immediately after Val-149 of the HBc gene. These constructs are shown schematically below.

| | |
|---|---|
| ICC-1438 | |
| MGISLLTEVETPIRNEWGCRCNDSSDELLGWLWGI-HBc(2-149) | SEQ ID NO:310 |
| ICC-1492 | |
| MGISLLTEVETPIRNEWGCRCNDSSDELLGWLWGI-HBc(2-149)-C | SEQ ID NO:311 |

It should be noted that to guard against translation initiation from the natural HBc initiator methionine, the codon for that residue was mutated to code for an isoleucine residue. Residues contributed by EcoRI (GI) and SacI (EL) restriction sites are underlined. The precore sequence is recited between the underlined EL residues and “-HBc(2-149)”.

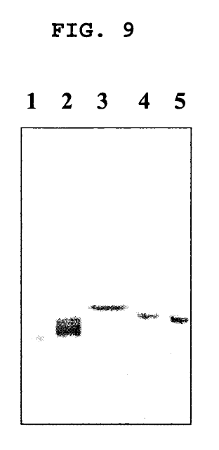

Analysis by SDS-PAGE as discussed elsewhere herein, showed that upon preparation, the ICC-1438 monomer construct was unstable (Lane 2) as compared to the ICC-1492 (Lane 3), with HBc-149 (Lane 1), ICC-1475 (Lane 4) and ICC-1473 (Lane 5) serving as additional molecular weight controls on the SDS-PAGE gel in FIG. 9. The instability of the ICC-1438 monomers was not evident using analytical gel filtration of particles.

Both ICC-1475 (FIG. 9, lane 4) and ICC-1473 (FIG. 9, lane 5) were expected to have slightly lower molecular weights than ICC-1438 and ICC-1492, because the former two contain the M2 epitope inserted directly into the immunodominant loop and therefore lack the precore sequence (SEQ ID NO: 310) present in ICC-1438 and ICC-1498. As expected, ICC-1492 was larger than ICC-1475 and ICC-1473; however, ICC-1438, which is identical to ICC-1492 save the C-terminal cysteine residue, is clearly not larger than ICC-1475 and ICC-1473 due to an apparent cleavage.

A construct conataining a M2 N-terminal extracellular sequence as discussed above linked to the HBc N-terminus (Domain I) or loop (Domain II) and also containing a M2 protein C-terminal sequence such as that of SEQ ID NO: 10 (see Table A) linked the loop (Domain II) or at the C-terminus (Domain IV) of HBc is also contemplated. Such a contemplated construct also contains at least one stabilizing C-terminal cysteine residue as discussed elssewhere herein.

### EXAMPLE 26

### Comparative Immunogenicities in Monkeys

The comparative immunogenicity of the particles expressed by V12.Pf3.1, formulated with either Seppic™ ISA-720 (Seppic Inc., Paris, France), Alhydrogel™ (Superfos, Denmark) as adjuvants, or unformulated (saline), was studied in Cynomolgus monkeys.

The Seppic™ ISA-720 formulation was prepared according to the manufacturers directions. Briefly, the ISA-720 and V12.Pf3.1 particles were mixed at 70:30 (w/w) ratio and vortexed, using a bench top vortexer, set at maximum power, for 1 minute. The Alhydroge™ formulation was prepared using an 8-fold excess of Alhydrogel™ (by weight) over V12.Pf3.1 particles, which was shown to be physically bound to the Alhydrogel™ prior to immunization.

Groups of two monkeys (one male and one female) were immunized with 20 μg V12.Pf3.1 particles as immunogenvia the intramuscular route. Animals were bled on days 0, 21, 42, 56 and 70, and sera analyzed for titers of anti-NANP antibody using an ELISA. The results, shown in Table 15, below, demonstrate the extremely high immunogenicity of V12.Pf3.1 particles when formulated with Seppic™ ISA-720 versus Alhydrogel™-formulated or unformulated material. The kinetics of the antibody response were more rapid when Seppic™ ISA-720 was used as the adjuvant, and the end-point titers were more than 100- and 1000-fold higher than for Alhydrogel™ and saline respectively.

TABLE 15

| | Antibody Titers at Stated Time (Days) | | | | |
|---|---|---|---|---|---|
| Adjuvant | Zero | 21 | 42 | 56 | 70 |
| Saline | Zero | 40 | 240 | 1,200 | 640 |
| Anhydrogel ™ | Zero | 2,880 | 1920 | 11,500 | 6400 |
| Seppic ™ | Zero | 81,920 | 348,160 | 26,000,000 | 1,920,000 |
| ISA-720 | | | | | |

### EXAMPLE 27

### T Cell Activation

Mice were immunized twice with V12.Pf3.1 particles in Seppic™ Montanide™ ISA-720. Spleen cells were removed and stimulated in the presence of various peptides. 10⁶ cells were incubated for 3 days in the presence of peptides: UTC (universal T epitope from P. falciparum; Seq 1N NO: 120), p85-100 peptide corresponding to HBc 85-100, NANP (B-cell epitope from V12.Pf3.1; NANPNVDP(NANP)₃, SEQ ID NO:22) in the presence of Staphylococcal enterotoxin B (SEB), or tissue culture medium (unstim). Interferon gamma production after 3 days was determined by ELISA.

The results shown in Table 16, below, indicate that immunizing with V12.Pf3.1 induces T-cells that recognize the UTC component of the protein, and drives them to a Th1 type response.

TABLE 16

| | IFN-γ | |
|---|---|---|
| Immunogen | (pg/ml) | S.D.* |
| UTC | 1600 | 750 |
| p85-100 | 350 | 30 |
| NANPNVDP(NANP)₃ | 370 | 50 |
| SEQ ID NO: 22 | | |
| SEB | 4300 | ND** |
| unstim | 900 | 1100 |

*S.D. = Standard Deviation
**ND = Not Done

Each of the patents and articles cited herein is incorporated by reference. The use of the article “a” or “an” is intended to include one or more.

The foregoing description and the examples are intended as illustrative and are not to be taken as limiting. Still other variations within the spirit and scope of this invention are possible and will readily present themselves to those skilled in the art.

What is claimed:

1. A recombinant chimer hepatitis B core (HBC) protein molecule up to about 515 amino acid residues in length that
(a) contains an HBc sequence of at least about 130 of the N-terminal 150 amino acid residues of the HBc molecule that include a peptide-bonded heterologous epitope or a heterologous linker residue for a conjugated epitope present in the HBc immunodominant loop, or a sequence of at least about 135 residues of the N-terminal 150 HBc amino acid residues,
(b) contains one to ten cysteine residues toward the C-terminus of the molecule from the C-terminal residue of the HBc sequence and within about 30 residues from the C-terminus of the chimer molecule [C-terminal cysteine residue(s)],
(c) contains a sequence of at least 5 amino acid residues from HBc position 135 to the HBc C-terminus,
said chimer molecules (i) containing no more than 20 percent conservatively substituted amino acid residues in the HBc sequence, (ii) self-assembling into particles that are substantially free of binding to nucleic acids on expression in a host cell, and said particles being more stable than are particles formed from an otherwise identical HBc chimer that lacks said C-terminal cysteine residue(s) or in which a C-terminal cysteine residue present in the chimer molecule is replaced by another residue.

2. The recombinant HBc chimer protein molecule according to claim 1 wherein said peptide-bonded heterologous epitope or a heterologous linker residue for a conjugated epitope is a heterologous epitope.

3. The recombinant HBc chimer protein molecule according to claim 2 wherein said heterologous epitope is a B cell epitope.

4. The recombinant HBc chimer protein molecule according to claim 3 that contains a second heterologous epitope peptide-bonded to one of amino acid residues 1-4 of HBc.

5. The recombinant HBc chimer protein molecule according to claim 3 wherein said B cell epitope is peptide-bonded at a position in the HBc sequence between amino acid residues 76 and 85, and at least 5 residues of the HBc sequence of positions 76 through 85 are present.

6. The recombinant HBc chimer protein molecule according to claim 5 wherein the HBc sequence between amino acid residues 76 and 85 is present, but interrupted by said B cell epitope.

7. The recombinant HBc chimer protein molecule according to claim 2 further including a peptide-bonded heterologous T cell epitope.

8. The recombinant HBc chimer protein molecule according to claim 7 wherein said T cell epitope is peptide-bonded to the C-terminal HBc amino acid residue.

9. The recombinant HBc chimer protein molecule according to claim 8 wherein said C-terminal cysteine residue(s) is present within five amino acid residues of the C-terminus of the HBc chimer protein molecule.

10. The recombinant HBc chimer protein molecule according to claim 1 wherein said chimer contains the uninterrupted HBc amino acid residue sequence of position 1 through at least position 140, plus a cysteine residue at the C-terminus of the HBc chimer protein molecule.

11. The recombinant HBc chimer protein molecule according to claim 10 wherein said chimer contains the uninterrupted HBc amino acid residue sequence of position 1 through position 149.

12. The recombinant HBc chimer protein molecule according to claim 1 wherein said chimer contains a heterologous linker residue for a conjugated epitope.

13. The recombinant HBc chimer protein molecule according to claim 12 wherein said heterologous linker residue for a conjugated epitope is peptide-bonded at a position in the HBc sequence between amino acid residues 76 and 85, and at least 4 residues of the HBc sequence of positions 76 through 85 are present.

14. The recombinant HBc chimer protein molecule according to claim 13 wherein the HBc sequence between amino acid residues 76 and 85 is present, but interrupted by said heterologous linker residue for a conjugated epitope.

15. The recombinant HBc chimer protein molecule according to claim 14 that contains the HBc amino acid residue sequence of position 1 through at least position 140, plus a single cysteine residue at the C-terminus.

16. The recombinant HBc chimer protein molecule according to claim 15 wherein said chimer contains the HBc amino acid residue sequence of position 1 through position 149.

17. The recombinant HBc chimer protein molecule according to claim 16 wherein said heterologous linker residue for a conjugated epitope is selected from the group consisting of a lysine, aspartic acid, glutamic acid, cysteine and a tyrosine residue.

18. A recombinant hepatitis B virus core (HBc) protein chimer molecule with a length of about 135 to about 515 amino acid residues that contains four peptide-linked amino acid residue sequence domains from the N-terminus that are denominated Domains I, II, III and IV, wherein
(a) Domain I comprises about 71 to about 100 amino acid residues whose sequence includes at least the sequence of the residues of position 5 through position 75 of HBc and optionally includes a heterologous epitope containing up to about 30 amino acid residues peptide-bonded to one of HBc residues 1-4;
(b) Domain II comprises about 5 to about 250 amino acid residues peptide-bonded to HBC residue 75 of Domain I in which (i) zero to all residues in a sequence of HBc positions 76 through 85 are present peptide-bonded to one to about 245 amino acid residues that are heterologous to HBc and constitute a heterologous epitope or a heterologous linker residue for a conjugated epitope or (ii) the sequence of HBc at positions 76 to 85 is present free from heterologous residues, or (iii) one or more of residues 76 to 85 is absent;
(c) Domain III is an HBc sequence from position 86 through position 135 peptide-bonded to residue 85 of Domain II; and
d) Domain IV comprises (i) zero through fourteen residues of a HBc amino acid residue sequence from position 136 through 149 peptide-bonded to the residue of position 135 of Domain III, (ii) one to ten cysteine residues [C-terminal cysteine residue(s)] within about 30 residues from the C-terminus of the chimer molecule, and (iii) zero to about 100 amino acid residues in a sequence heterologous to HBc from position 150 to the C-terminus, with the proviso that Domain IV contain at least 6 amino acid residues including said one to ten cysteine residues of (ii),
said chimer self-assembling into particles on expression in a host cell, said particles being substantially free of binding to nucleic acids and more stable than are particles formed from an otherwise identical HBc chimer that lacks said C-terminal cysteine residue(s) or in which a C-terminal cysteine residue present in the chimer molecule is replaced by another residue, and having an amino acid residue sequence in which no more than about 10 percent of the amino acid residues are substituted in the HBc sequence of the chimer.

19. The recombinant HBc chimer protein molecule according to claim 18 that contains two heterologous epitopes.

20. The recombinant HBc chimer protein molecule according to claim 19 wherein said two heterologous epitopes are present in Domains I and II, II and IV or I and IV.

21. The recombinant HBc chimer protein molecule according to claim 19 wherein one of said two heterologous epitopes is a B cell epitope.

22. The recombinant HBc chimer protein molecule according to claim 19 wherein one of said two heterologous epitopes is a T cell epitope.

23. The recombinant HBc chimer protein molecule according to claim 19 wherein one of said two heterologous epitopes is a B cell epitope and the other is a T cell epitope.

24. The recombinant HBc chimer protein molecule according to claim 18 wherein said Domain I includes a heterologous epitope peptide-bonded to one of HBc residues 1-4.

25. The recombinant HBC chimer protein molecule according to claim 24 wherein said heterologous epitope of Domain II is a B cell epitope.

26. The recombinant HBc chimer protein molecule according to claim 25 wherein said sequence heterologous to HBc from position 150 to the C-terminus is a T cell epitope peptide-bonded to one of HBc residues 140-149.

27. The recombinant HBc chimer protein molecule according to claim 18 wherein said heterologous linker residue for a conjugated epitope or a heterologous epitope is a heterologous epitope.

28. The recombinant HBc chimer protein molecule according to claim 27 wherein said heterologous epitope comprises up to about 245 amino acid residues.

29. The recombinant HBc chimer protein molecule according to claim 28 wherein said heterologous epitope is a B cell epitope.

30. The recombinant HBc chimer protein molecule according to claim 27 wherein said heterologous epitope contains 6 to about 50 amino acid residues.

31. The recombinant HBc chimer protein molecule according to claim 27 wherein said heterologous epitope contains 20 to about 30 amino acid residues.

32. The recombinant HBC chimer protein molecule according to claim 27 wherein said Domain IV comprises 1 to about 5 cysteine residues within about 30 residues from the C-terminus of the chimer molecule.

33. The recombinant HBc chimer protein molecule according to claim 27 wherein the HBc sequence between amino acid residues 76 and 85 is present, but interrupted by said heterologous epitope.

34. The recombinant HBc chimer protein molecule according to claim 18 wherein said C-terminal cysteine residue is located within about five amino acid residues of the C-terminus of the chimer protein molecule.

35. The recombinant HBc chimer protein molecule according to claim 18 wherein said sequence heterologous to HBc from position 150 to the C-terminus is a T cell epitope peptide-bonded to one of HBc residues 140-149.

36. The recombinant HBc chimer protein molecule according to claim 18 wherein said heterologous linker residue for a conjugated epitope or a heterologous epitope is a heterologous linker residue for a conjugated epitope.

37. The recombinant HBc chimer protein molecule according to claim 36 wherein said heterologous linker residue for a conjugated epitope is selected from the group consisting of a lysine, aspartic acid, glutamic acid, cysteine and a tyrosine residue.

38. The recombinant HBc chimer protein molecule according to claim 37 that contains a single cysteine residue at the C-terminus of the HBc chimer protein molecule.

39. The recombinant HBc chimer protein molecule according to claim 18 wherein said chimer contains the uninterrupted HBc amino acid residue sequence through at least position 140.

40. The recombinant HBc chimer protein molecule according to claim 39 wherein said uninterrupted HBc amino acid residue sequence includes residue 1.

41. The recombinant HBc chimer protein molecule according to claim 39 wherein said uninterrupted HBc amino acid residue sequence includes residue 149.

42. A recombinant hepatitis B virus core (HBc) protein chimer molecule with a length of about 175 to about 240 amino acid residues that contains four peptide-linked amino acid residue sequence domains from the N-terminus that are denominated Domains I, II, III and IV, wherein
(a) Domain I comprises about the sequence of the residues of position 1 through position 75 of HBc;
(b) Domain II comprises about 5 to about 55 amino acid residues peptide-bonded to HBc residue 75 of Domain I in which at least 4 residues in a sequence of HBc positions 76 through 85 are present peptide-bonded to 6 to about 50 amino acid residues that are heterologous to HBc and constitute a heterologous epitope;
(c) Domain III is an HBc sequence from position 86 through position 135 peptide-bonded to residue 85 of Domain II; and
d) Domain IV comprises (i) 5 through fourteen residues of a HBc amino acid residue sequence from position 136 through 149 peptide-bonded to the residue of position 135 of Domain III, (ii) a cysteine residue [C-terminal cysteine residue] within about 30 residues from the C-terminus of the chimer molecule, and (iii) zero to about 50 amino acid residues in a sequence heterologous to HBc from position 150 to the C-terminus,
said chimer self-assembling into particles on expression in a host cell that exhibit a ratio of absorbance at 280 nm to 260 nm of about 1.2 to about 1.6 and are more stable than are particles formed from an otherwise identical HBc chimer molecule that lacks said C-terminal cysteine residue or in which a C-terminal cysteine residue present in the chimer molecule is replaced by another residue, and having an amino acid residue sequence in which no more than about 5 percent of the amino acid residues are substituted in the HBc sequence of the chimer.

43. The recombinant HBc chimer protein molecule according to claim 42 wherein said heterologous epitope of Domain II is a B cell epitope.

44. The recombinant HBc chimer protein molecule according to claim 43 wherein said heterologous epitope contains 15 to about 50 amino acid residues.

45. The recombinant HBc chimer protein molecule according to claim 43 wherein said heterologous epitope contains 20 to about 30 amino acid residues.

46. The recombinant HBc chimer protein molecule according to claim 43 wherein the HBc sequence between amino acid residues 76 and 85 is present, but interrupted by said heterologous epitope.

47. The recombinant HBc chimer protein molecule according to claim 43 wherein said B cell epitope is an amino acid sequence present in a pathogen selected from the group consisting of Streptococcus pneumonia, Cryptosporidium parvum, HIV, foot-and-mouth disease virus, influenza virus, Yersinia pestis, Haemophilus influenzae, Moraxella catarrhalis, Porphyromonas gingivalis, Trypanosoma cruzi, Plasmodium falciparum, Plasmodium vivax, Plasmodium berghi, Plasmodium yoelli, Streptococcus sobrinus, Shigella flexneri, RSV, Plasmodium Entamoeba histolytica, Schistosoma japonicum, Schistosoma mansoni, bovine inhibin and ebola virus.

48. The recombinant HBc chimer protein molecule according to claim 43 wherein said sequence heterologous to HBc from position 150 to the C-terminus is a T cell epitope peptide-bonded to one of HBc residues 140-149.

49. The recombinant HBc chimer protein molecule according to claim 48 wherein said T cell epitope is from the organism against which a contemplated chimer is to be used as an immunogen.

50. The recombinant HBc chimer protein molecule according to claim 43 wherein said C-terminal cysteine residue is located within about five amino acid residues of the C-terminus of the chimer protein molecule.

51. An immunogenic particle comprised of recombinant hepatitis B core (HBc) chimeric protein molecules, said chimeric protein (i) displaying one or more immunogenic epitopes at the N-terminus, HBc immunogenic loop or C-terminus, or (ii) having a heterologous linker residue for a conjugated epitope in the HBc immunogenic loop, and containing a cysteine residue at or near the C-terminus, said particle being substantially free of nucleic acid binding and exhibiting enhanced stability relative to particles comprised of otherwise identical proteins that are free of said cysteine residue.

52. The immunogenic particle according to claim 51 that exhibits a 280/260 absorbance ratio of about 1.2 to about 1.7.

53. The immunogenic particle according to claim 51 whose recombinant HBc chimeric protein displays an immunogenic epitope at the N-terminus.

54. The immunogenic particle according to claim 51 whose recombinant HBc chimeric protein displays an immunogenic epitope at the C-terminus.

55. The immunogenic particle according to claim 51 whose recombinant HBc chimeric protein displays an immunogenic epitope in the immunogenic loop.

56. The immunogenic particle according to claim 1 whose recombinant HBc chimeric protein displays a B cell immunogenic epitope.

57. The immunogenic particle according to claim 51 whose recombinant HBc chimeric protein displays a T cell immunogenic epitope.

58. The immunogenic particle according to claim 51 whose recombinant HBc chimeric protein displays separate B cell and T cell immunogenic epitopes.

59. The immunogenic particle according to claim 51 whose recombinant HBc chimeric protein has a heterologous linker residue for a conjugated epitope in the HBc immunogenic loop.

60. The immunogenic particle according to claim 59 wherein said heterologous linker residue for a conjugated epitope is selected from the group consisting of a lysine, aspartic acid, glutamic acid, cysteine and a tyrosine residue.

61. The immunogenic particle according to claim 60 wherein said heterologous linker residue for a conjugated epitope is conjugated to a hapten.

62. The immunogenic particle according to claim 61 wherein said hapten is an oligosaccharide.

63. An immunogenic particle comprised of a plurality of recombinant chimeric hepatitis B core (HBc) protein molecules;
said recombinant chimeric HBc protein molecules having a length of up to about 515 amino acid residues that
(a) contain a HBc sequence of at least about 130 of the N-terminal 150 amino acid residues of the HBc molecule that include a peptide-bonded heterologous epitope or a heterologous linker residue for a conjugated epitope present in the HBc immunodominant loop, or a sequence of at least about 135 residues of the N-terminal 150 HBc amino acid residues,
(b) contain one to ten cysteine residues toward the C-terminus of the molecule from the C-terminal residue of the HBc sequence and within about 30 residues from the C-terminus of the chimer molecule [C-terminal cysteine residue(s)],
(c) contain a sequence of at least 6 amino acid residues from HBc position 135 to the HBc C-terminus,
said chimer molecules containing no more than 10 percent conservatively substituted amino acid residues in the HBc sequence, and
said particles being substantially free of binding to nucleic acids, and being more stable than are particles formed from an otherwise identical HBc chimer that lacks said C-terminal cysteine residue(s) or in which a C-terminal cysteine residue present in the chimer molecule is replaced by another residue, and having an amino acid residue sequence in which no more than about 20 percent of the amino acid residues are substituted in the HBc sequence of the chimer.

64. The immunogenic particle according to claim 63 that exhibit a ratio of absorbance at 280 nm to 260 nm of about 1.4 to about 1.6.

65. The immunogenic particle according to claim 63 wherein the length of said recombinant chimeric HBc protein molecules is about 175 to about 240 amino acid residues.

66. The immunogenic particle according to claim 63 wherein said peptide-bonded heterologous epitope or a heterologous linker residue for a conjugated epitope is a heterologous epitope.

67. The immunogenic particle according to claim 66 wherein said heterologous epitope is a B cell epitope.

68. The immunogenic particle according to claim 63 wherein the length of said recombinant chimeric HBC protein molecules is up to about 435 amino acid residues.

69. The immunogenic particle according to claim 63 that contains a second heterologous epitope peptide-bonded to one of amino acid residues 1-4 of HBc.

70. The immunogenic particle according to claim 68 wherein said B cell epitope is peptide-bonded at a position in the HBc sequence between amino acid residues 76 and 85, and at least 5 residues of the HBc sequence of positions 76 through 85 are present.

71. The immunogenic particle according to claim 70 wherein the HBc sequence between amino acid residues 76 and 85 is present, but interrupted by said B cell epitope.

72. The immunogenic particle according to claim 68 further including a peptide-bonded heterologous T cell epitope.

73. The immunogenic particle according to claim 72 wherein said T cell epitope is peptide-bonded to the C-terminal HBc amino acid residue.

74. The immunogenic particle according to claim 73 wherein said C-terminal cysteine residue(s) is present within five amino acid residues of the C-terminus of the HBc chimer protein molecule.

75. The immunogenic particle according to claim 63 wherein said recombinant chimeric HBc protein molecules have a length of about 135 to about 515 amino acid residues and contains four peptide-linked amino acid residue sequence domains from the N-terminus that are denominated Domains I, II, III and IV, wherein
(a) Domain I comprises about 71 to about 100 amino acid residues whose sequence includes at least the sequence of the residues of position 5 through position 75 of HBc and optionally includes a heterologous epitope containing up to about 30 amino acid residues peptide-bonded to one of HBc residues 1-4;
(b) Domain II comprises about 5 to about 250 amino acid residues peptide-bonded to HBc residue 75 of Domain I in which (i) zero to all of the residues in a sequence of HBc positions 76 through 85 are present peptide-bonded to one to about 245 amino acid residues that are heterologous to HBc and constitute a heterologous epitope or a heterologous linker residue for a conjugated epitope or (ii) the sequence of HBc at positions 76 to 85 is present free from heterologous residues;
(c) Domain III is an HBc sequence from position 86 through position 135 peptide-bonded to residue 85 of Domain II; and
d) Domain IV comprises (i) zero through fourteen residues of a HBc amino acid residue sequence from position 136 through 149 peptide-bonded to the residue of position 135 of Domain III, (ii) one to ten cysteine residues [C-terminal cysteine residue(s)] within about 30 residues from the C-terminus of the chimer molecule, and (iii) zero to about 100 amino acid residues in a sequence heterologous to HBc from position 150 to the C-terminus, with the proviso that Domain IV contain at least 6 amino acid residues including said one to ten ceyteine residues of (ii), said chimeric HBc protein having an amino acid residue sequence in which no more than about 10 percent of the amino acid residues are substituted in the HBc sequence.

76. The immunogenic particle according to claim 75 that contains a heterologous linker residue for a conjugated epitope in Domain II and further includes a hapten linked to said heterologous linker residue.

77. The immunogenic particle according to claim 76 wherein said hapten is a B cell immunogen.

78. The immunogenic particle according to claim 63 wherein said recombinant chimeric HBc protein molecules have a length of about 175 to about 240 amino acid residues and contain four peptide-linked amino acid residue sequence domains from the N-terminus that are denominated Domains I, II, III and IV, wherein
(a) Domain I comprises about the sequence of the residues of position 1 through position 75 of HBc;
(b) Domain II comprises about 5 to about 55 amino acid residues peptide-bonded to HBc residue 75 of Domain I in which at least 4 residues in a sequence of HBc positions 76 through 85 are present peptide-bonded to 6 to about 50 amino acid residues that are heterologous to HBC and constitute a heterologous epitope;
(c) Domain III is an HBc sequence from position 86 through position 135 peptide-bonded to residue 85 of Domain II; and
d) Domain IV comprises (i) 5 through fourteen residues of a HBc amino acid residue sequence from position 136 through 149 peptide-bonded to the residue of position 135 of Domain III, (ii) one to about five cysteine residues [C-terminal cysteine residue] within about 30 residues from the C-terminus of the chimer molecule, and (iii) zero to about 50 amino acid residues in a sequence heterologous to HBc from position 150 to the C-terminus,
said particles exhibiting a ratio of absorbance at 280 nm to 260 nm of about 1.4 to about 1.6, and said chimeric HBc protein having an amino acid residue sequence in which no more than about 5 percent of the amino acid residues are substituted in the HBc sequence.

79. A vaccine or inoculum comprising an immunogenic effective amount of immunogenic particles dissolved or dispersed in a pharmaceutically acceptable diluent, wherein said immunogenic particles are comprised of a plurality of recombinant chimeric hepatitis B core (HBc) protein molecules in which said recombinant chimeric HBc protein molecules have a length of up to about 515 amino acid residues that
(a) contain a sequence of at least about 130 of the N-terminal 150 amino acid residues of the HBc molecule that include a peptide-bonded heterologous epitope or a heterologous linker residue for a conjugated epitope present in the HBC immunodominant loop, or a sequence of at least about 135 residues of the N-terminal 150 HBC amino acid residues,
(b) contain one to ten cysteine residues toward the C-terminus of the molecule from the C-terminal residue of the HBc sequence and within about 30 residues from the C-terminus of the chimer molecule [C-terminal cysteine residue(s)],
(c) contain a sequence of at least 6 amino acid residues from HBc position 135 to the HBc C-terminus,
said chimer molecules containing no more than 20 percent conservatively substituted amino acid residues in the HBc sequence, and
said particles being substantially free of binding to nucleic acids, and being more stable than are particles formed from an otherwise identical HBc chimer that lacks said C-terminal cysteine residue(s) or in which a C-terminal cysteine residue present in the chimer molecule is replaced by another residue.

80. The vaccine or inoculum according to claim 79 wherein said recombinant chimeric HBc protein molecules have a length of about 135 to about 515 amino acid residues and contains four peptide-linked amino acid residue sequence domains from the N-terminus that are denominated Domains I, II, III and IV, wherein
(a) Domain I comprises about 71 to about 100 amino acid residues whose sequence includes at least the sequence of the residues of position 5 through position 75 of HBc and optionally includes a heterologous epitope containing up to about 30 amino acid residues peptide-bonded to one of HBc residues 1-4;
(b) Domain II comprises about 5 to about 250 amino acid residues peptide-bonded to HBc residue 75 of Domain I in which (i) at least 4 residues in a sequence of HBc positions 76 through 85 are present peptide-bonded to one to about 245 amino acid residues that are heterologous to HBc and constitute a heterologous epitope or a heterologous linker residue for a conjugated epitope or (ii) the sequence of HBc at positions 76 to 85 is present free from heterologous residues;
(c) Domain III is an HBc sequence from position 86 through position 135 peptide-bonded to residue 85 of Domain II; and
d) Domain IV comprises (i) zero through fourteen residues of a HBc amino acid residue sequence from position 136 through 149 peptide-bonded to the residue of position 135 of Domain III, (ii) one to ten cysteine residues [C-terminal cysteine residue(s)] within about 30 residues from the C-terminus of the chimer molecule, and (iii) zero to about 100 amino acid residues in a sequence heterologous to HBc from position 150 to the C-terminus, with the proviso that Domain IV contain at least 6 amino acid residues including said one to ten cysteine residues of (ii), said recombinant chimeric HBc protein molecules having an amino acid residue sequence in which no more than about 5 percent of the amino acid residues are substituted in the HBc sequence.

81. The vaccine or inoculum according to claim 80 that contains a heterologous linker residue for a conjugated epitope in Domain II and further includes a hapten linked to said heterologous linker residue.

82. The vaccine or inoculum according to claim 79 wherein said recombinant chimeric HBc protein molecules have a length of about 175 to about 240 amino acid residues and contain four peptide-linked amino acid residue sequence domains from the N-terminus that are denominated Domains I, II, III and IV, wherein
(a) Domain I comprises about the sequence of the residues of position 1 through position 75 of HBc;
(b) Domain II comprises about 5 to about 55 amino acid residues peptide-bonded to HBc residue 75 of Domain I in which at least 4 residues in a sequence of HBc positions 76 through 85 are present peptide-bonded to 6 to about 50 amino acid residues that are heterologous to HBc and constitute a heterologous epitope;
(c) Domain III is an HBc sequence from position 86 through position 135 peptide-bonded to residue 85 of Domain II; and
d) Domain IV comprises (i) 5 through fourteen residues of a HBc amino acid residue sequence from position 136 through 149 peptide-bonded to the residue of position 135 of Domain III, and (ii) zero to about 50 amino acid residues in a sequence heterologous to HBc from position 150 to the C-terminus,
said particles exhibiting a ratio of absorbance at 280 nm to 260 nm of about 1.4 to about 1.6.

83. The vaccine or inoculum according to claim 79 that is adapted for parenteral administration.

84. The vaccine or inoculum according to claim 79 that is adapted for mucosal immunization.

85. The vaccine or inoculum according to claim 79 wherein said recombinant chimeric HBc protein molecule particles are present in an attenuated strain of S. typhi, S. typhimurium or a S. typhimurium-E. coli hybrid.

86. The vaccine or inoculum according to claim 79 wherein said recombinant chimeric HBc protein molecule particles are present in plant tissue.

87. The vaccine or inoculum according to claim 79 that further includes an adjuvant.

88. The vaccine or inoculum according to claim 87 wherein said adjuvant is alum.

89. The vaccine or inoculum according to claim 87 wherein said adjuvant is a small molecule selected from the group consisting of a muramyl dipeptide, 7-substituted-8-oxo- or 8-sulfo-guanosine derivative, monophosphoryl lipid A, aluminum or calcium salts.

90. The vaccine or inoculum according to claim 87 wherein said adjuvant is an oil that is emulsified with said immunogenic particles and said pharmaceutically acceptable diluent.

91. The vaccine or inoculum according to claim 90 wherein said emulsion is an water-in-oil emulsion having a water phase and an oil phase.

92. The vaccine or inoculum according to claim 90 wherein said emulsion is an oil-in-water emulsion having a water phase and an oil phase.

93. The vaccine or inoculum according to claim 92 wherein the oil phase of said emulsion comprises squalene.

94. The vaccine or inoculum according to claim 92 wherein the oil phase of said emulsion comprises squalane.

95. The vaccine or inoculum according to claim 90 wherein the water and oil phases of said emulsion are emulsified by an emulsifying agent that is a sorbitan or mannide C₁₂-C₂₄ fatty acid ester.

96. The vaccine or inoculum according to claim 95 wherein said emulsifying agent is a mannide C₁₂-C₂₄ fatty acid ester.

97. The vaccine or inoculum according to claim 96 wherein said C₁₂-C₂₄ fatty acid of said mannide C₁₂-C₂₄ fatty acid ester is oleic acid.

98. A nucleic acid that encodes a recombinant HBc protein molecule according to claim 1, or a variant, analog or complement thereof.

99. A nucleic acid that encodes a recombinant HBc protein molecule according to claim 18, or a variant, analog or complement thereof.

100. A nucleic acid that encodes a recombinant HBc protein molecule according to claim 42, or a varient, analog or complement thereof.

101. A recombinant nucleic acid molecule that comprises a vector operatively linked to a nucleic acid segment defining a gene that encodes a recombinant HBc protein molecule according to claim 1, or a varient, analog or complement thereof, and a promoter suitable for driving the expression of the gene in a compatible host organism.

102. A recombinant nucleic acid molecule that comprises a vector operatively linked to a nucleic acid segment defining a gene that encodes a recombinant HBc protein molecule according to claim 18, or a varient, analog or complement thereof, and a promoter suitable for driving the expression of the gene in a compatible host organism.

103. A recombinant nucleic acid molecule that comprises a vector operatively linked to a nucleic acid segment defining a gene that encodes a recombinant HBc protein molecule according to claim 42, or a varient, analog or complement thereof, and a promoter suitable for driving the expression of the gene in a compatible host organism.

104. A host cell transformed with a recombinant nucleic acid molecule according to claim 101.

105. The transformed host cell according to claim 104 wherein said host cell is selected from the group consisting of CHO, VERO or COS cells, E. coli, S. cerivisiae, Pichia pastoris typhi, S. typhimurium and a S. typhimurium-E. coli hybrid.

106. A host cell transformed with a recombinant nucleic acid molecule according to claim 102.

107. The transformed host cell according to claim 106 wherein said host cell is selected from the group consisting of CHO, VERO or COS cells, E. coli, S. cerivisiae, Pichia pastoris typhi, S. typhimurium and a S. typhimurium-E. coli hybrid.

108. A host cell transformed with a recombinant nucleic acid molecule according to claim 102.

109. The transformed host cell according to claim 108 wherein said host cell is selected from the group consisting of CHO, VERO or COS cells, E. coli, S. cerivisiae, Pichia pastoris typhi, S. typhimurium and a S. typhimurium-E. coli hybrid.

110. A method of inducing an immune response in an inoculated host animal that comprises the steps of inoculating a host animal with a vaccine or inoculum according to claim 79, and maintaining that inoculated animal for a time period sufficient for that animal to develop an immune response.

111. A method of inducing an immune response in an inoculated host animal that comprises the steps of inoculating a host animal with a vaccine or inoculum according to claim 80, and maintaining that inoculated animal for a time period sufficient for that animal to develop an immune response.

112. A method of inducing an immune response in an inoculated host animal that comprises the steps of inoculating a host animal with a vaccine or inoculum according to claim 82, and maintaining that inoculated animal for a time period sufficient for that animal to develop an immune response.

113. A method of inducing an immune response in an inoculated host animal that comprises the steps of inoculating a host animal with a vaccine or inoculum according to claim 87, and maintaining that inoculated animal for a time period sufficient for that animal to develop an immune response.

114. A method of inducing an immune response in an inoculated host animal that comprises the steps of inoculating a host animal with a vaccine or inoculum according to claim 88, and maintaining that inoculated animal for a time period sufficient for that animal to develop an immune response.

115. A method of inducing an immune response in an inoculated host animal that comprises the steps of inoculating a host animal with a vaccine or inoculum according to claim 92, and maintaining that inoculated animal for a time period sufficient for that animal to develop an immune response.
